(19) United States
(12) Patent Application Publication
(10) Pub. No.: US 2020/0087625 A1
(43) Pub. Date: Mar. 19, 2020

(54) METHODS FOR ISOLATING, EXPANDING AND ADMINISTERING CANCER SPECIFIC CD8+ T CELLS

(71) Applicants: Dennis A. Carson, La Jolla, CA (US); Tomoko Hayashi, San Diego, CA (US); Kun-Liang Guan, La Jolla, CA (US); Toshiro Moroishi, Kumamoto (JP)

(72) Inventors: Dennis A. Carson, La Jolla, CA (US); Tomoko Hayashi, San Diego, CA (US); Kun-Liang Guan, La Jolla, CA (US); Toshiro Moroishi, Kumamoto (JP)

(21) Appl. No.: 16/495,083

(22) PCT Filed: Mar. 30, 2018
(86) PCT No.: PCT/US18/25522

§ 371 Date: Sep. 17, 2019

Related U.S. Application Data
(60) Provisional application No. 62/479,057, filed on Mar. 30, 2017.

(51) Int. Cl. C12N 5/0783; A61K 35/17; A61P 35/04

(52) U.S. Cl. CPC C12N 5/0636; A61P 35/04; A61K 35/17

(57) ABSTRACT

Methods to isolate and expand tumor specific CD8+ T cells, and the use thereof, are provided. Also provided are method of using TLR7 agonists.

### CROSS-REFERENCE TO RELATED APPLICATIONS

This application claims the benefit of the filing date of U.S. application Ser. No. 62/479,057, filed on Mar. 30, 2017, the disclosure of which is incorporated by reference herein.

### STATEMENT OF GOVERNMENT RIGHTS

This invention was made with government support under grant number R35CA196878 awarded by the National Institutes of Health. The government has certain rights in the invention.

### BACKGROUND

Treatment with autologous CD8 positive patient-specific T cells has produced long term remissions in some patients with melanoma and other cancers (Rosenberg and Restifo, 2015). However, it has been very difficult to identify, and expand such cells. Usually, the so-called Tumor Infiltrating Lymphocytes (TILs) must be isolated from surgical or biopsy specimens, and expanded for weeks in tissue culture until a sufficiently large number of cells can be recovered for re-infusion (Rosenberg et al., 2008) (FIG. 1). For these reasons, autologous T cell therapy of cancer has not been widely adopted.

Recently, the Rosenberg group at the NCI was successful in identifying neo-antigen specific T cells in the peripheral blood of some melanoma patients, using a combination of surface markers and functional assessment of reactivity to tumor antigens (Gros et al., 2016). However, the number of patients in which such cells can be recovered and expanded is meager. Accordingly, there is an unmet and critical need for simple and practical methods to increase the numbers of TILs in the blood, to demonstrate their tumor reactivity, and to expand them for re-administration.

### SUMMARY

A major goal of cancer immunotherapy is the expansion and/or reactivation of cytotoxic CD8⁺ T cell responses against malignant cells. Poorly immunogenic tumor cells evade host immunity and grow even in the presence of an intact immune system, but the complex mechanisms regulating tumor immunogenicity have not been elucidated. Here, in three different murine syngeneic tumor models (B16, SCC7, and 4T1), it was demonstrated that loss of the Hippo pathway kinases LATS1/2 (large tumor suppressor 1 and 2) in tumor cells inhibits tumor growth. Tumor regression by LATS1/2 deletion requires adaptive immune responses, and LATS1/2 deficiency enhances tumor vaccine efficacy. Mechanistically, LATS1/2-null tumor cells secrete nucleic acid-rich extracellular vesicles (EVs), which induce a type I interferon response via the Toll-like receptors-MYD88/TRIF pathway. LATS1/2 deletion in tumors thus improves tumor immunogenicity, leading to tumor destruction by enhancing anti-tumor immune responses. These observations uncover a role of the Hippo pathway in modulating tumor immunogenicity and demonstrate a proof of concept for targeting LATS1/2 in cancer immunotherapy.

In one embodiment, a protocol that allows maximizing the therapeutic effect of anti-PD1 therapy for the clonal expansion of tumor specific CD8+ T cells in TILs and spleen by local administration of an immune stimulating agent (e.g., a TLR7 or TLR9 agonist), is provided. This clonal expansion is a great predictor of efficacy of anti-PD1 therapy and hence a guiding indicator patients should be administered/treated with this therapy.

In one embodiment, a patient with cancer is administered specific drugs that promote the release of immunogenic tiny EVs from cancer cells, in the absence of cytotoxicity, which in turn induces clonal expansion of tumor specific CD8+ T cells, without knowing the exact antigen specific for the tumor cells. In one embodiment, after drug therapy, tumor specific CD8+ T cells are expanded in tissue culture, which can be made more efficient by purification of activated CD8+ T cells and by co-culture of the T cells with the immunogenic EVs derived from the blood of the same patient in the presence or absence of feeder cells. In one embodiment, the treatment of cancer patients with anti-PD1 and other checkpoint inhibitors should be undertaken specifically in those subjects who have circulating clonal T cells after drug treatment, and at the time when both EVs and TIL concentrations in the blood reach maximal levels. In one embodiment, antigen presenting cells, like dendritic cells and macrophages, release exosomes (EVs) containing antigen and immune stimulating DNA/RNA which may function as “artificial antigen presenting cells” that travel through the lymphatics to distant sites, in order to initiate a good immune response.

In one embodiment, a method to isolate and expand cancer-specific CD8+ T cells is provided. In one embodiment, the method includes administering to a mammal having a tumor an effective amount of a composition comprising an agent that promotes the release of extracellular vesicles from tumor cells; and collecting from the mammal extracellular vesicles and immune cells including tumor specific CD8+ T cells. In one embodiment, the composition comprises a TLR7 or TLR9 agonist. In one embodiment, the extracellular vesicles and the immune cells are collected from blood of the mammal. In one embodiment, the extracellular vesicles and the immune cells are collected contemporaneously. In one embodiment, the agent inhibits or inactivates LATS1 and/or LATS2. In one embodiment, the method further includes culturing the immune cells to expand and/or activate cancer-specific CD8+ T cells. In one embodiment, the collected extracellular vesicles are cultured with the immune cells. In one embodiment, the collected extracellular vesicles are less than about 0.5 microns in diameter. In one embodiment, the collected extracellular vesicles are isolated. In one embodiment, in the isolated extracellular vesicles are less than about 0.5 microns in diameter. In one embodiment, the cultured and expanded cancer-specific CD8+ T cells are isolated from non-cancer specific CD8+ T cells. In one embodiment, the method further includes culturing the immune cells in the presence of feeder cells. In one embodiment, the method further includes collecting the expanded, or activated and expanded, cancer-specific CD8+ T cells. In one embodiment, the composition is orally or parenterally administered or by intrapulmonary routes. In one embodiment, the composition is administered to the tumor by direct injection. In one embodiment, the composition is administered systemically using liposomes, antibodies or other targeting mechanisms. In one embodiment, the mammal is subjected to chemotherapy before the immune cells are collected. In one embodiment, the mammal is subjected to chemotherapy after the immune cells are collected. In one embodiment, the method further includes isolating the cultured cancer-specific CD8+ T cells. In one embodiment, the cancer-specific CD8+ T cells are administered to the mammal. In one embodiment, the mammal is subjected to chemotherapy after the cancer-specific CD8+ T cells are administered. In one embodiment, wherein the therapy is anti-PD therapy. In one embodiment, the therapy is a checkpoint inhibitor therapy. In one embodiment, the inhibitor comprises pembrolizumab, nivolumab, atezolizumab, avelumab, durvalumab, or ipilimumab.

In one embodiment, a method to isolate and expand cancer-specific CD8+ T cells is provided. In one embodiment, the method includes collecting from a mammal having a tumor extracellular vesicles and immune cells including tumor specific CD8+ T cells; culturing the immune cells and enriching for cancer-specific CD8+ T cells; and culturing the enriched cancer-specific CD8+ T cells and the extracellular vesicles. In one embodiment, the mammal is subjected to chemotherapy before the immune cells are collected. In one embodiment, the mammal is subjected to chemotherapy after the immune cells are collected. In one embodiment, the method further includes isolating the cultured cancer-specific CD8+ T cells. In one embodiment, the cancer-specific CD8+ T cells are administered to the mammal. In one embodiment, the mammal is subjected to chemotherapy after the cancer-specific CD8+ T cells are administered. In one embodiment, wherein the therapy is anti-PD therapy. In one embodiment, the therapy is a checkpoint inhibitor therapy. In one embodiment, the inhibitor comprises pembrolizumab, nivolumab, atezolizumab, avelumab, durvalumab, or ipilimumab.

In one embodiment, a method to enhance the immunogenicity of tumor cells is provided. In one embodiment, the method includes modifying ex vivo tumor cells of a mammal to provide for tumor cells that do not express or have reduced expression of LAT1 and/or LAT2; and administering to the mammal an amount of the modified cells effective to enhance the immune response to the tumor in the mammal.

Although direct injection of toll-like receptor 7 (TLR7) agonists into primary tumors can induce tumor-specific oligoclonal T cell responses whose magnitude correlates with therapeutic efficacy, tumors are not always accessible to local therapy. Herein below, it is demonstrated that a single systemic administration of a phospholipid conjugated TLR7 agonist can also expand tumor-specific cytotoxic T cells that are shared by different animals. The expansion can be achieved without causing apparent toxicity. Similar technology combining immune repertoire analysis and immunomodulatory drugs can help to guide the development of optimal immunotherapeutic regimens in cancer patients.

### BRIEF DESCRIPTION OF THE FIGURES

FIG. 1. Current protocol for TIL isolation and expansion. The procedure in the current TIL, therapy requires collection of surgical specimens. Circulating tumor specific T cells can be collected from peripheral blood, however, the frequency of such cells is very low. Because pan T cell stimulators, e.g IL-2 and OKT3, are used to expand T cells, proliferation of non-specific T cells may overcome expansion of tumor specific clones.

FIGS. 2A-2F. Combination therapy with the TLR7 agonist 1V270 and systemic anti-PD-1 agent increases activated CD8⁺ population in TILs and spleens. (A-F) 1V270 increased CD8⁺ population in TILs. C3H mice (n=5-8/group) were implanted with SCC7 and carcinoma cells 1A. Tumors and spleens were harvested on day 21 and T cells in TILs or spleens were analyzed by flow cytometry. (A and B) Tumor infiltrating CD8⁺ T cells were gated on CD45⁺CD3⁺CD8⁺ populations. Number of CD8⁺ T cells (A) and IFNγ⁺CD8⁺ cells (B) per tumor volume (mm³) were calculated and plotted. (C) Representative immunohistochemistry images of the tumors (day 21) stained for CD8 (red) and DAPI (blue). Scale bar: 20 μm. (D) Number of IFNγ⁺CD8⁺ T cells in spleens. Bars indicate mean±SEM. Each dot represents an individual animal and bars indicate mean±SEM in A, B and D. P*<0.05, **P<0.01 (Kruskal-Wallis test with Dunn's post hoc test), n=5-8/group. (E) Tumor volumes at the injected sites were plotted against the log of the number of IFNγ⁺CD8⁺ T cells in the tumor microenvironment. Significant negative correlation was demonstrated by Spearman correlation test. (F) The tumor volumes (day 21, injected side) were plotted against the log of the numbers of IFNγ⁺CD8⁺ T cells in the spleen.

FIGS. 3A-3E. Systemic anti-PD-1 agent treatment or combination treatment increased TCR clonality of CD8⁺ T cells. (A-D) SCC7-bearing mice (n=4/group) were treated as described in FIG. 2. Tumors and spleens were harvested on day 21 and CD8⁺ T cells were isolated using MACSMicroBead. RNA was isolated and next-generation sequencing was performed. (A) Representative data of TCR repertoire clonality of CD8⁺ T cells. The X and Y-axes show the combination of V and J genes (TRAV and TRAJ families), and the Z-axis shows their frequency of usage. (B and C) Clonality indices (1-normalized Shannon index) in injected and distant uninjected tumors (B) and spleens (C). Higher value of clonality index reflects TCR clonal expansions. Closed and open symbols indicate injected and uninjected tumors, respectively. (D) Percentage of common TCR clones in injected, uninjected tumors and spleens. (E) The tumor volumes on day 21 were plotted against the log of % common TCR clones. n=16.

FIGS. 4A-4G. Loss of LATS1/2 in tumors inhibits tumor growth in vivo (A) Equal numbers of WT or LATS1/2 dKO B16-OVA cells were transplanted into C57BL/6 mice and tumor growth was monitored after the indicated times. Data are represented as mean±SEM; n=8 tumors for each group. ***p<0.001, two-way ANOVA test. (B) C57BL/6 mice were injected with WT or LATS1/2 dKO B16-OVA melanoma and tumor weight was determined 20 days after transplantation. Data are represented as mean±SEM; n=6 tumors for WT, n=8 tumors for LATS1/2 dKO (note that two of the tumors were completely rejected). **p<0.01, Mann-Whitney test.

(C) Kaplan-Meier tumor-free survival curves for mice injected with WT or LATS1/2 dKO B16-OVA cells are shown (n=14 mice for each group). ***p<0.001, log-rank test. (D) WT or two independent clones of LATS1/2 dKO SCC7 cells were transplanted into C3H/HeOu mice and tumor growth was monitored after the indicated times. Data are represented as mean±SEM; n=8 tumors for each group. p values were determined using two-way ANOVA test, comparing each group to WT group. ***p<0.001.
(E) Kaplan-Meier tumor-free survival curves for mice injected with WT or LATS1/2 dKO SCC7 cells are shown (n=4 mice for each group). p values were determined using log-rank test, comparing each group to WT group. **p<0.01.
(F) BALB/c mice were injected with WT or LATS1/2 dKO 4T1 cells and primary tumor weight was determined 28 days after transplantation. (G) WT or LATS1/2 dKO 4T1 cells were transplanted into the mammary fat pad of BALB/c mice and lung metastasis of the primary tumor was determined 28 days after transplantation. Normal lung tissue was stained with black India ink, whereas tumor nodules remain white. The gross appearance of the lungs (left panel) and tumor nodules on lungs (right panel) were examined. Data are represented as mean±SEM; n=16 tumors (F) and n=8 mice (G) for each group. ***p<0.001, Mann-Whitney test.

FIGS. 5A-5G. LATS1/2 deletion in tumors stimulates host adaptive immunity and enhances tumor vaccine efficacy (A) WT or LATS1/2 dKO B16-OVA melanoma cells were injected into C57BL/6 mice and tumor growth was monitored after the indicated times (left panel). For co-injection experiments, WT and LATS1/2 dKO cells were injected into opposite flanks in the same mouse (right panel). “WT [with LATS1/2 dKO(#1)]” (blue line) indicates WT tumor growth, and “LATS1/2 dKO(#1) [with WT]” (yellow line) indicates LATS1/2 dKO tumor growth, in the co-injected mice. Data are represented as mean±SEM; n=8 tumors for WT or LATS1/2 dKO group, n=6 tumors for each co-injectioned group. p value was determined using two-way ANOVA test, comparing WT [with LATS1/2 dKO(#1)] group to WT group. ***p<0.001. (B) C57BL/6 mice were immunized intradermally at the base of the tail with equal numbers of irradiated WT or LATS1/2 dKO B16-OVA cells (or PBS control). 12 days after immunization, mice were challenged with WT B16-OVA melanoma and tumor growth was monitored after the indicated times. Data are represented as mean±SEM; n=8 tumors for each group. PBS, phosphate buffered saline.

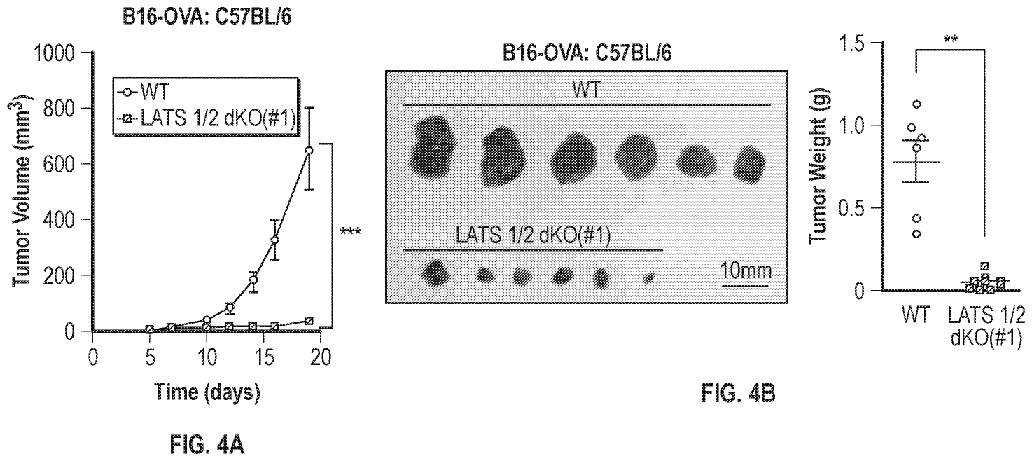

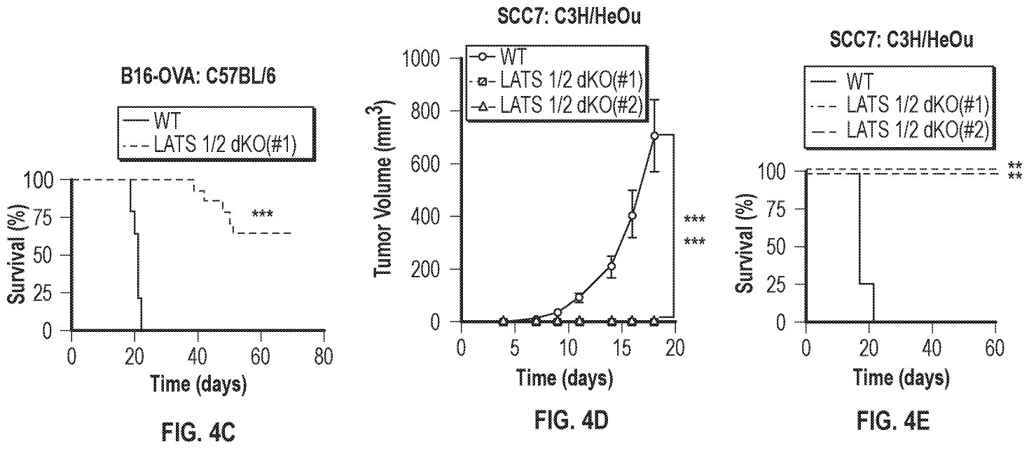

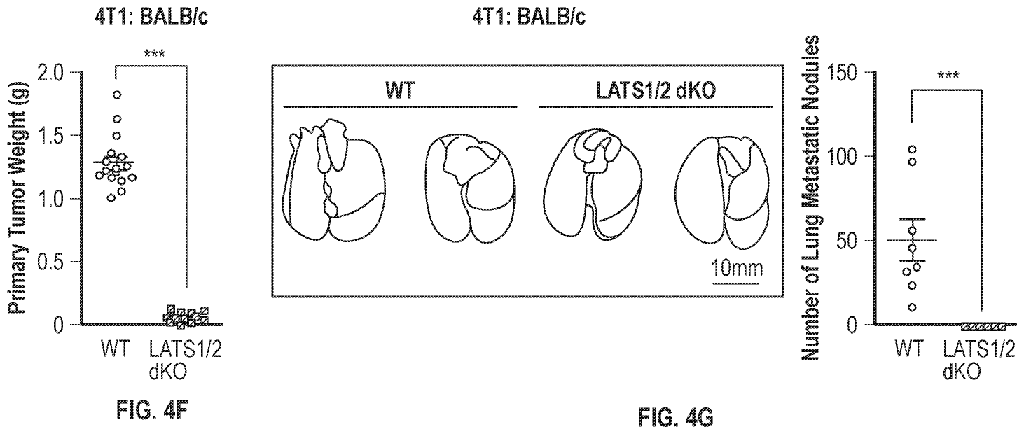

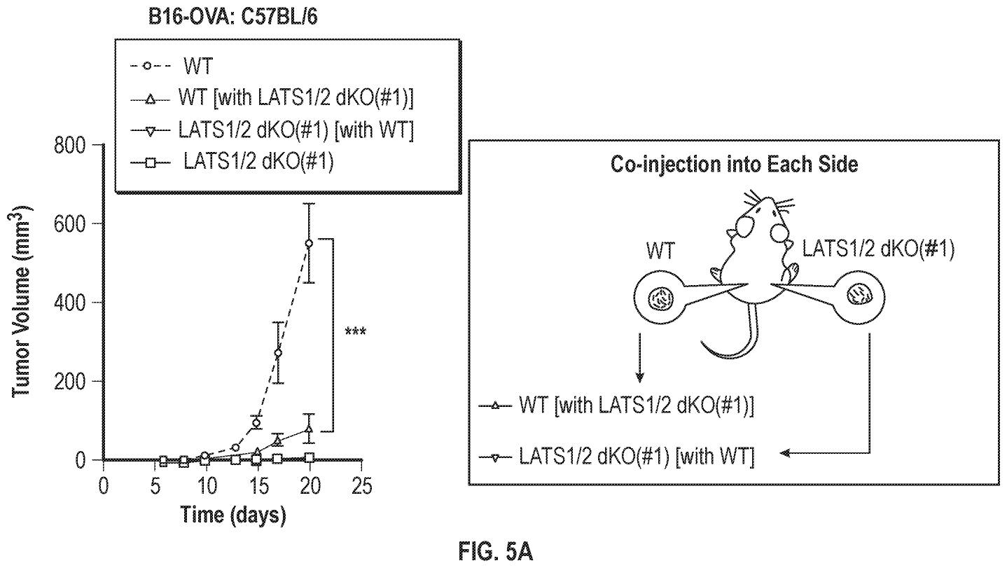

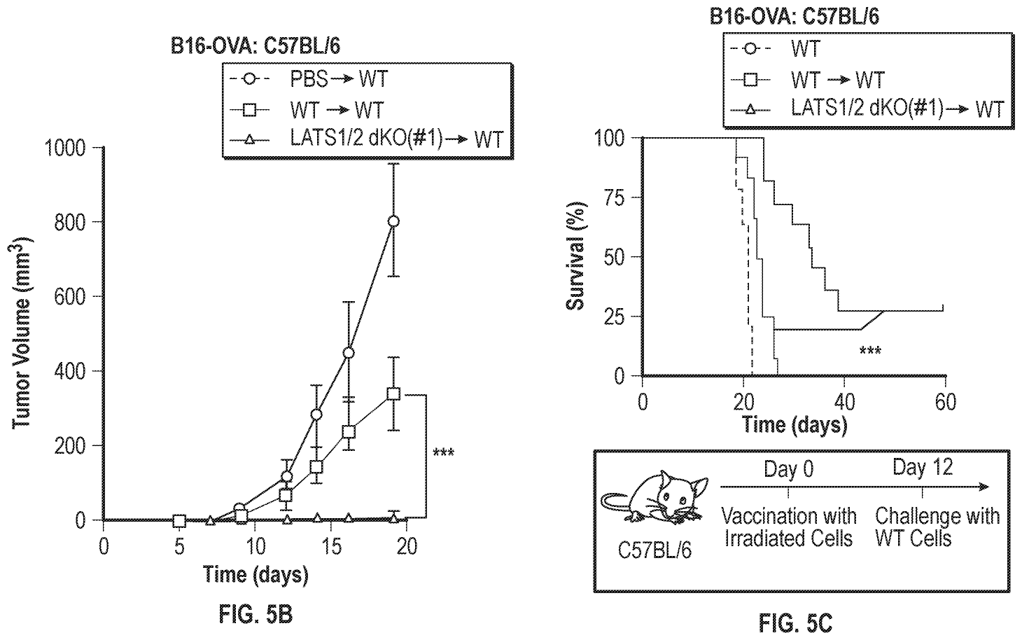

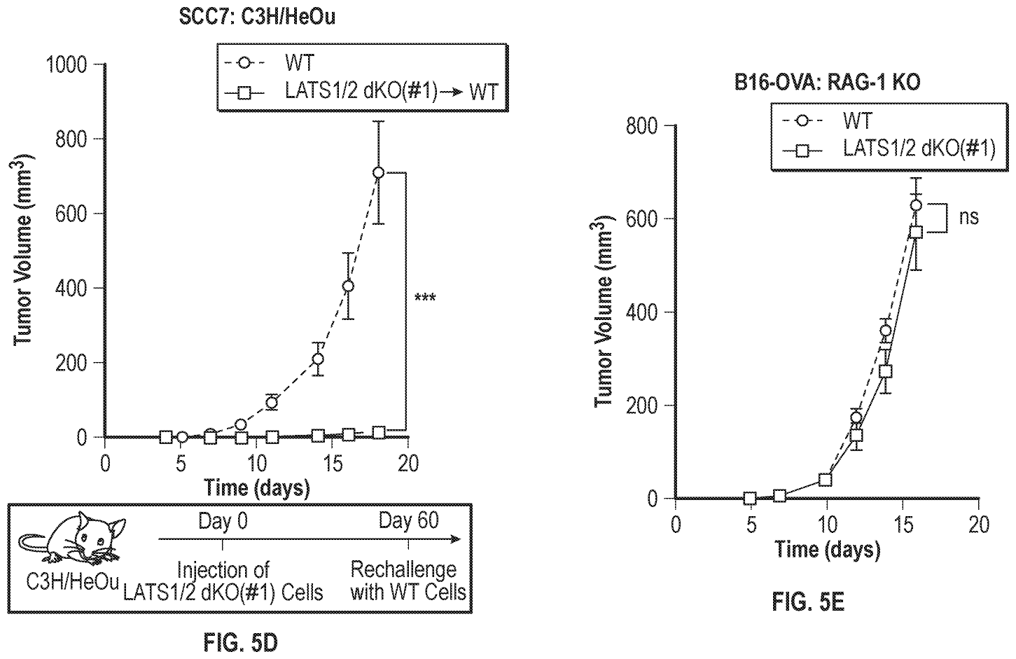

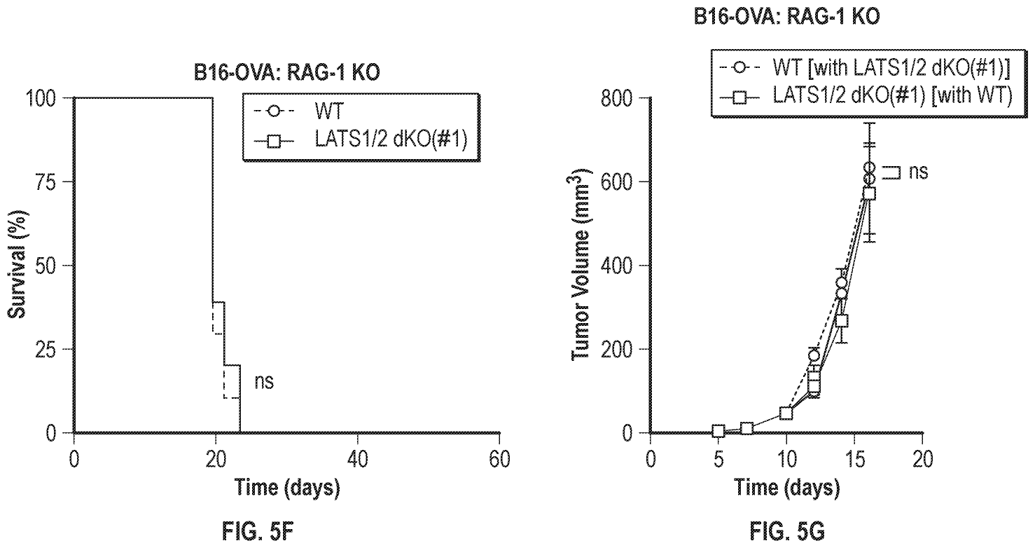

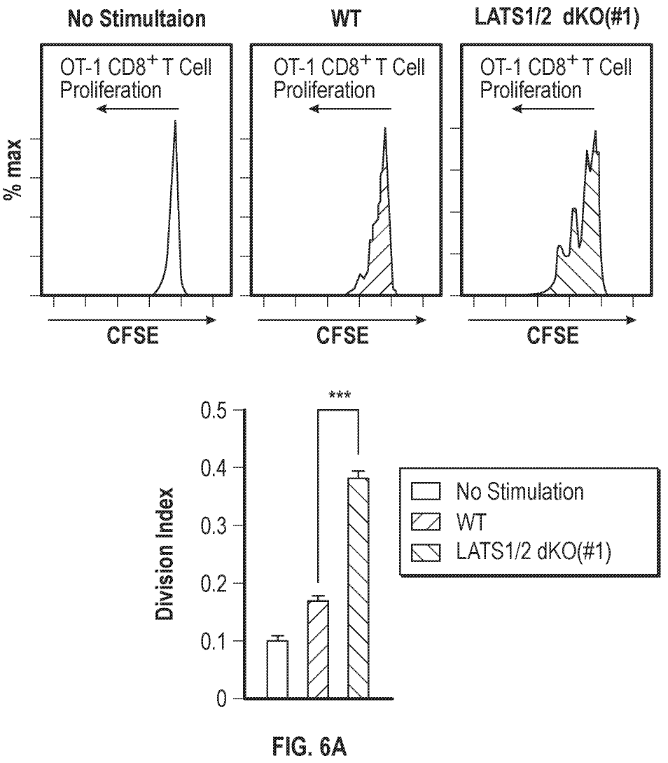

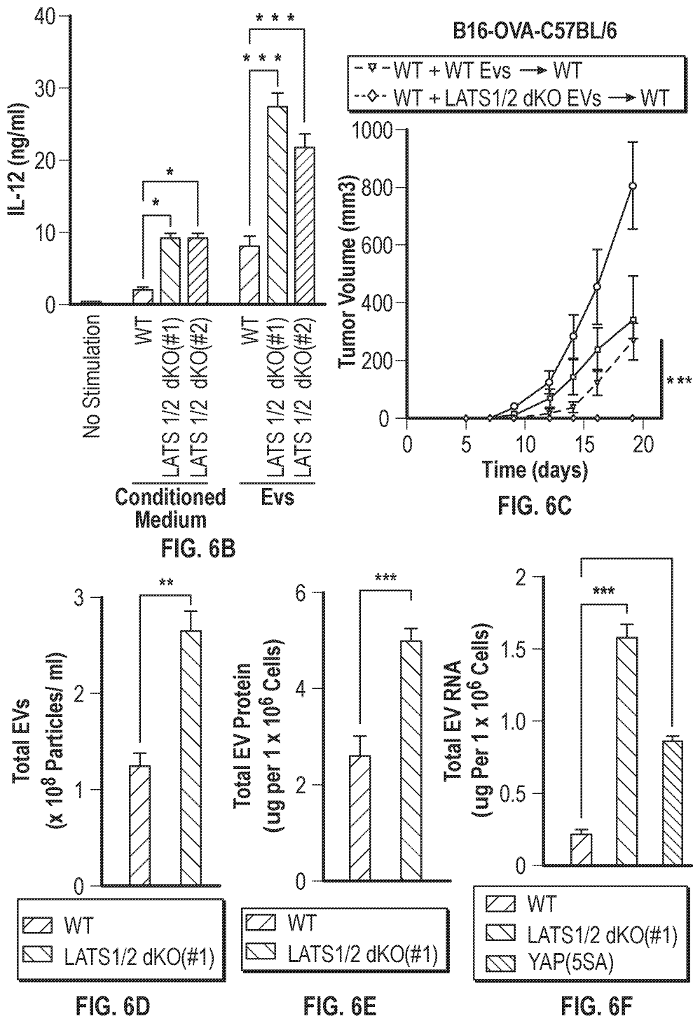

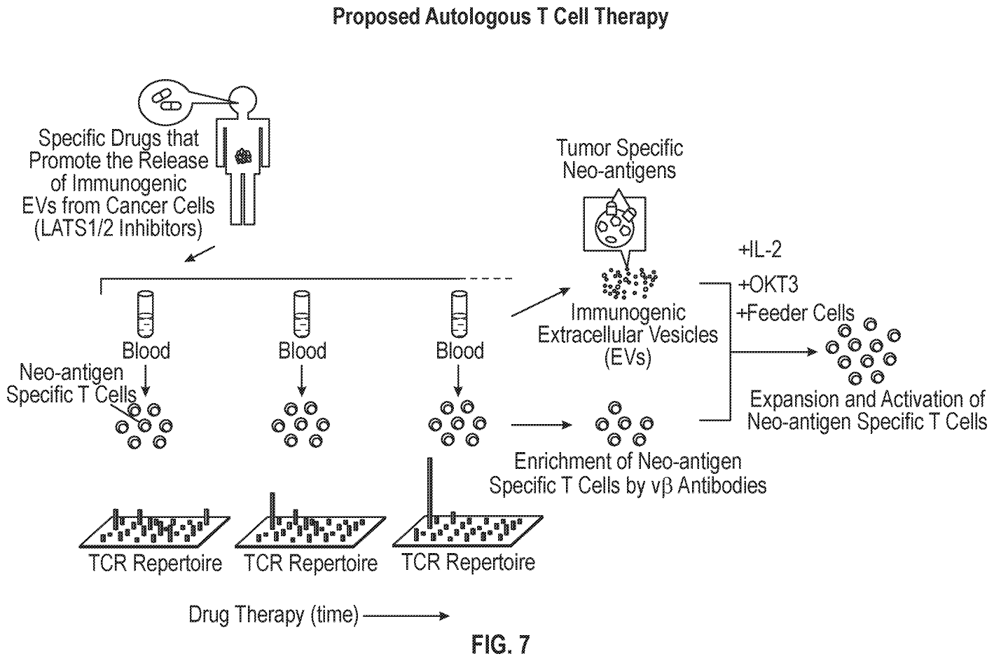

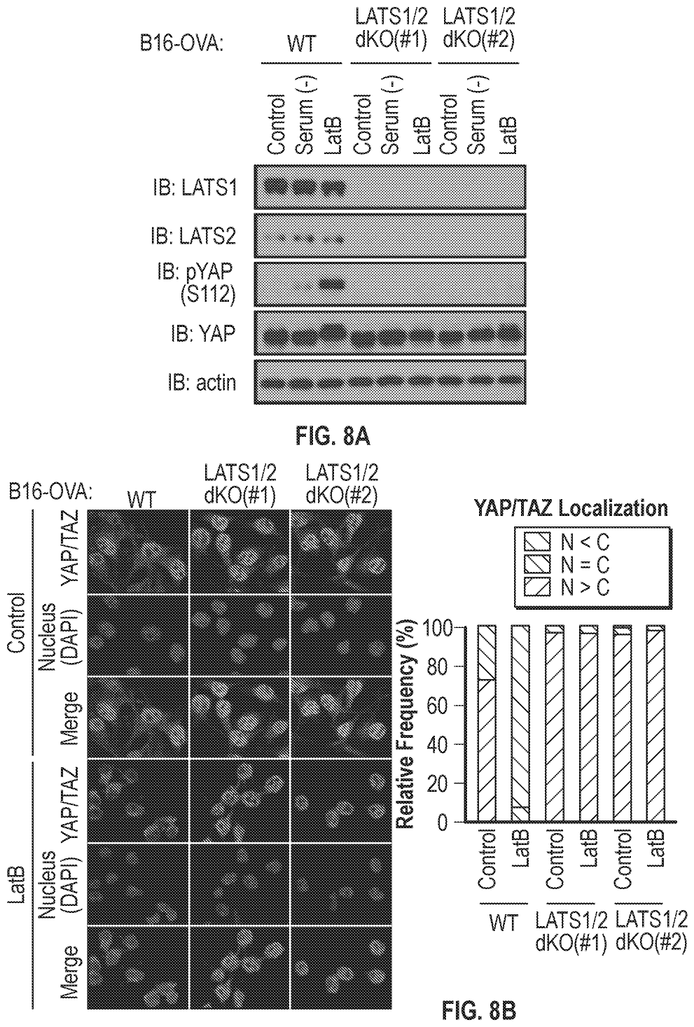

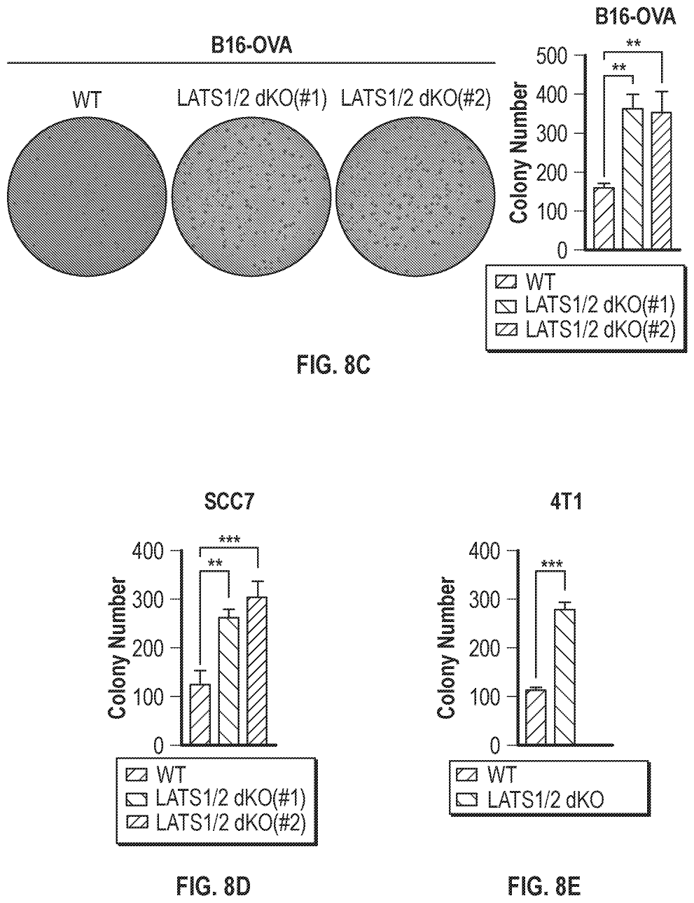

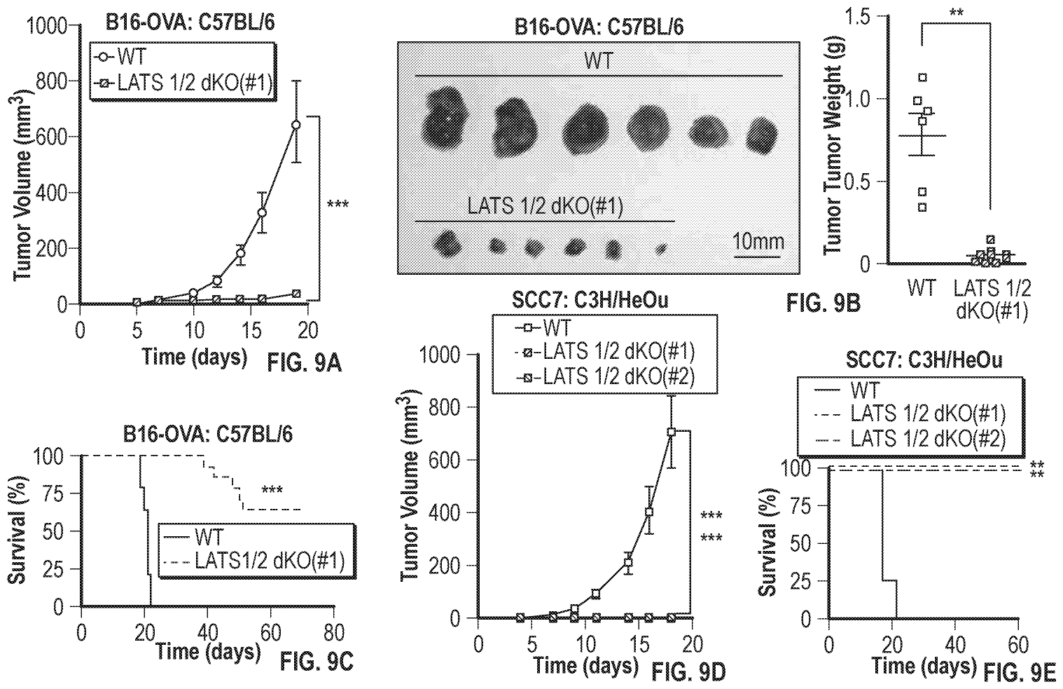

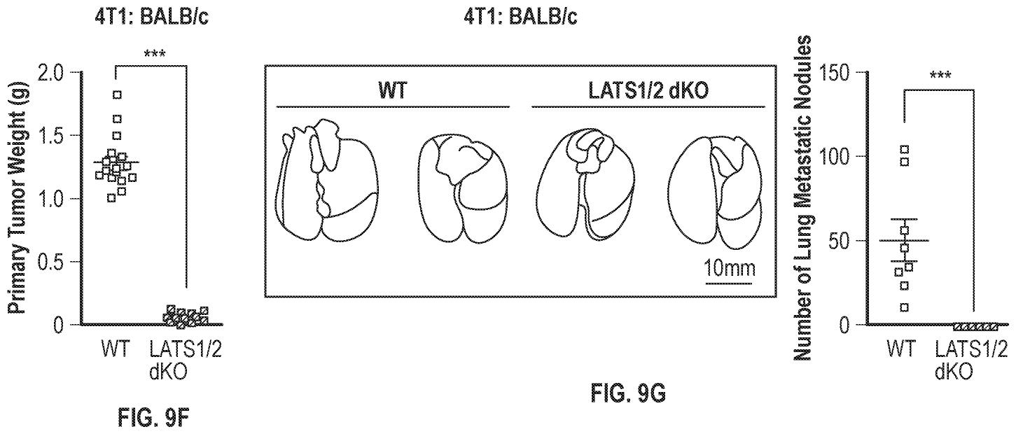

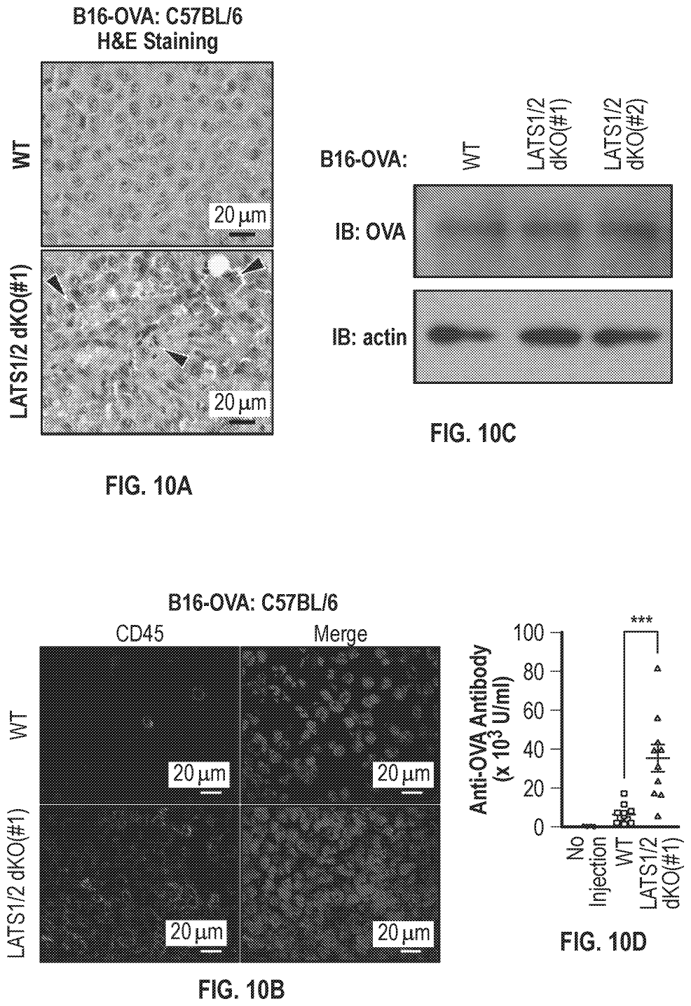

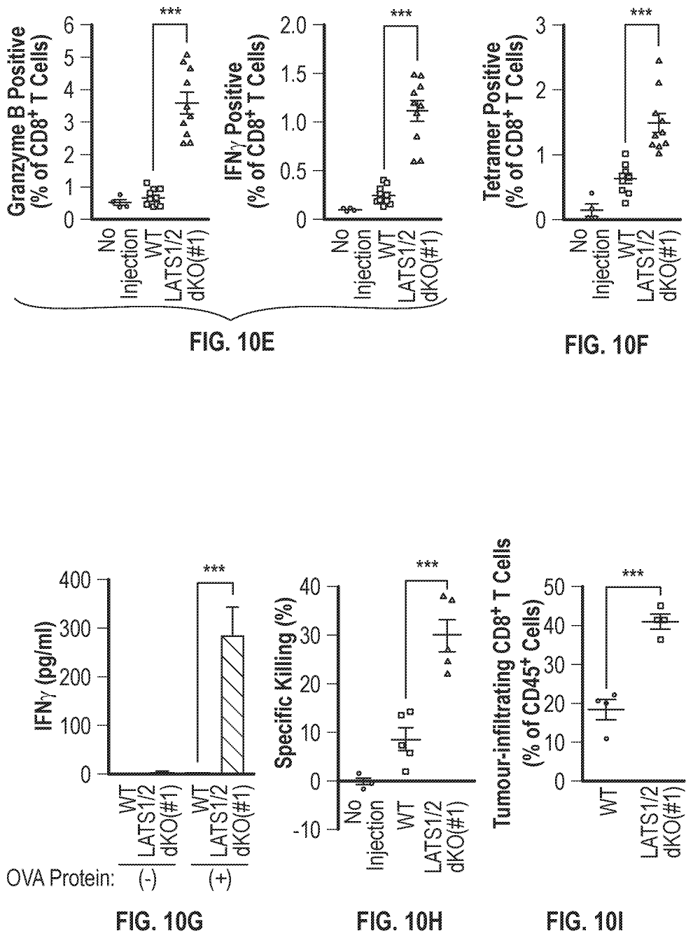

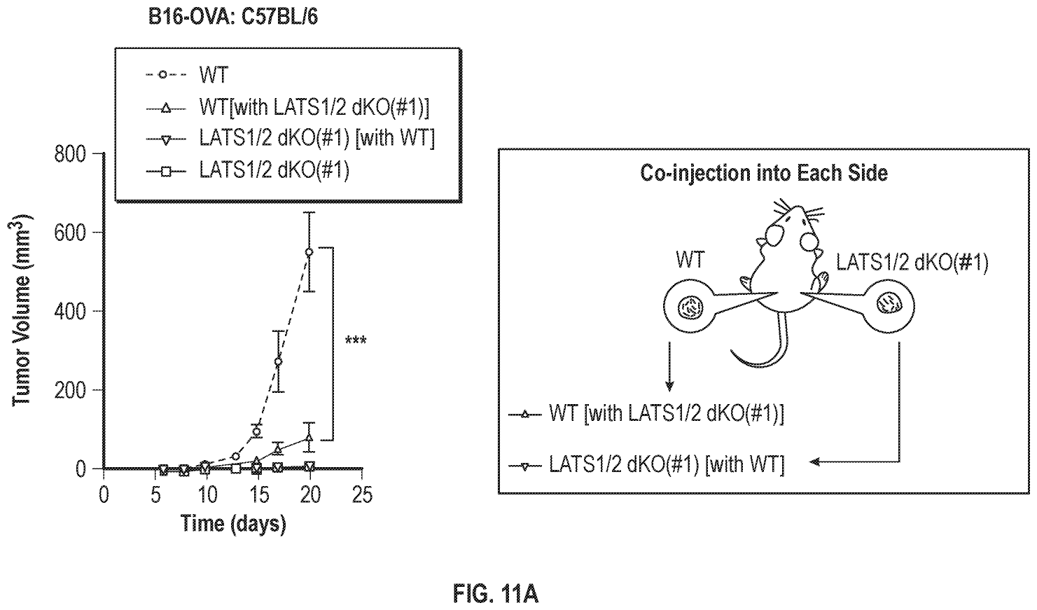

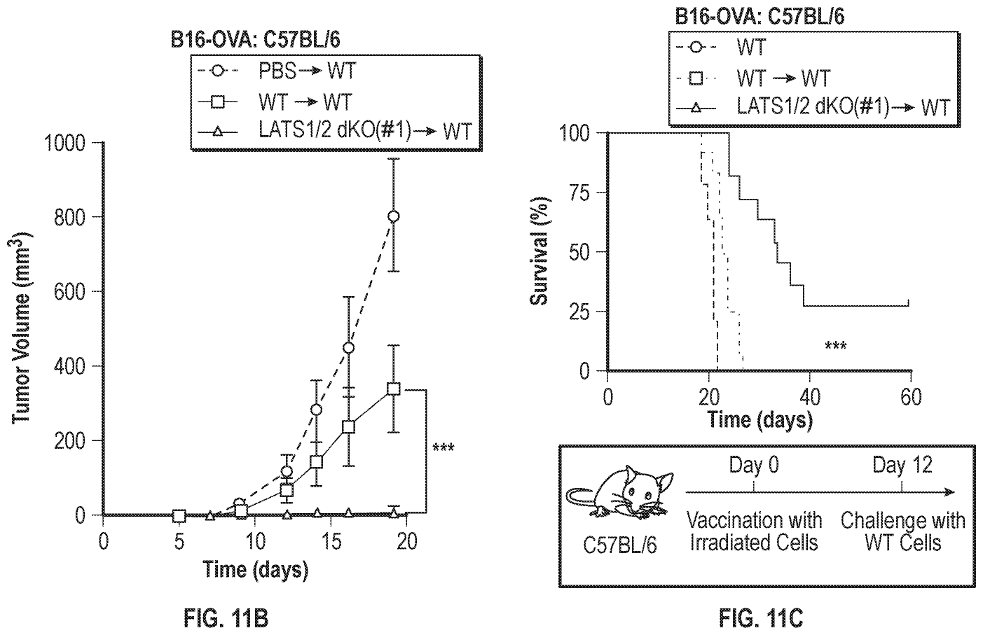

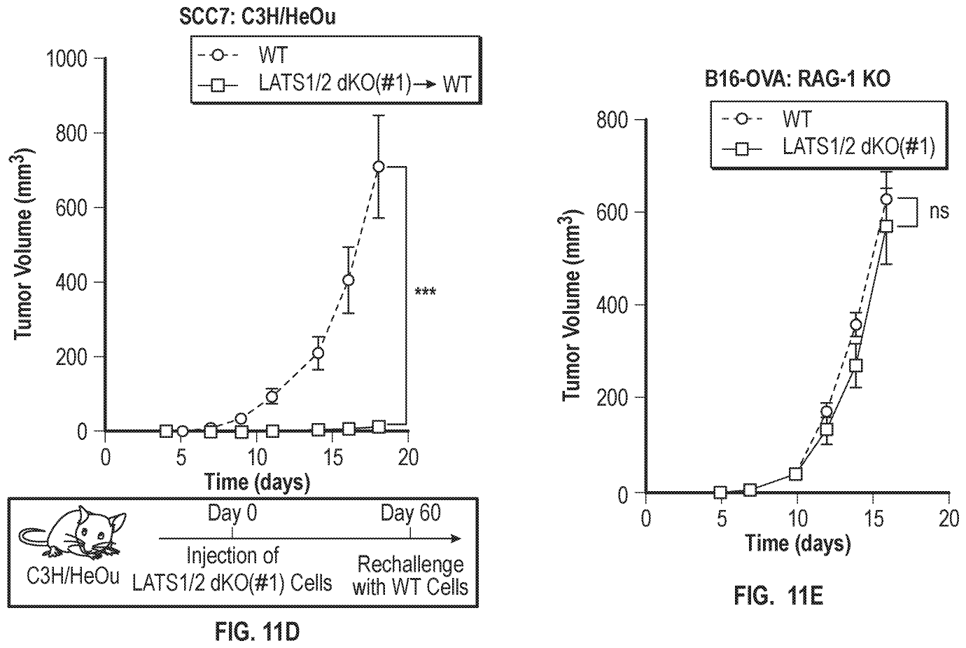

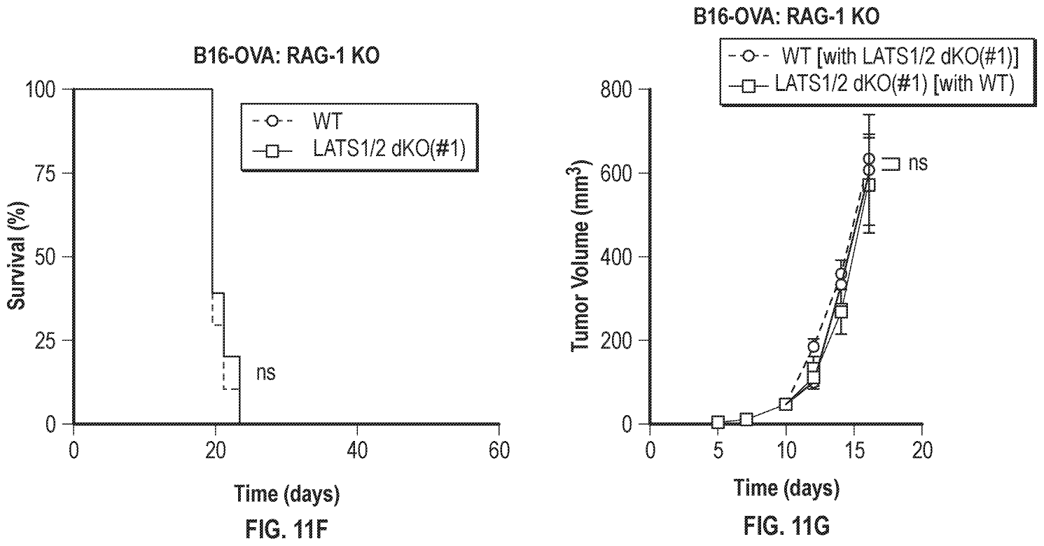

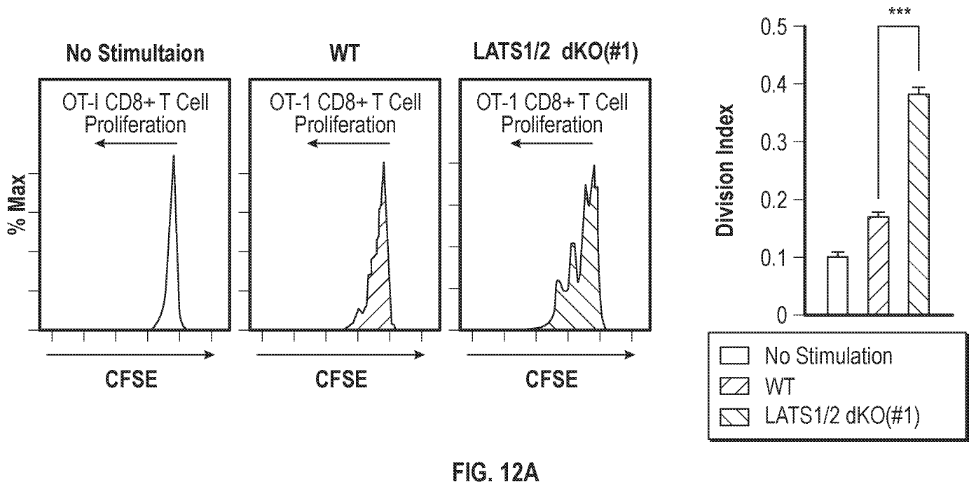

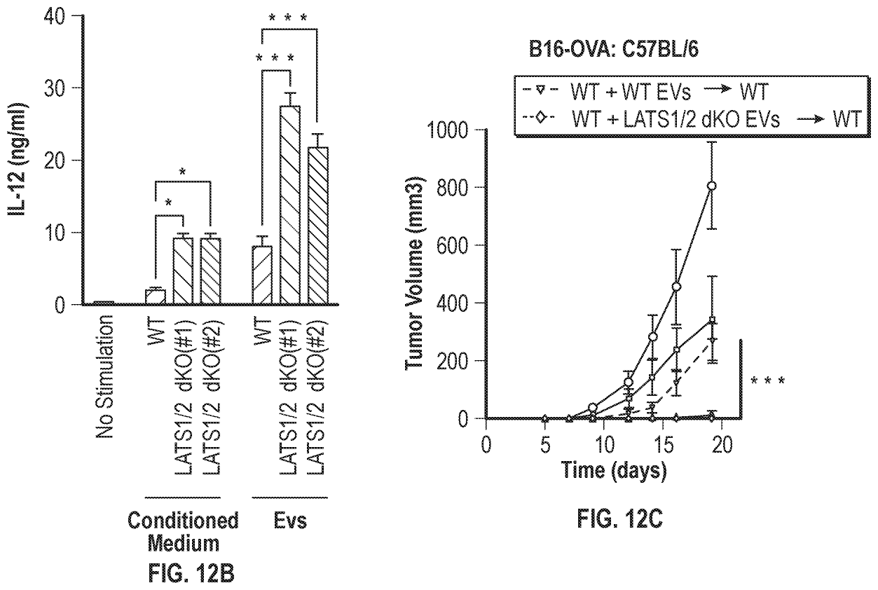

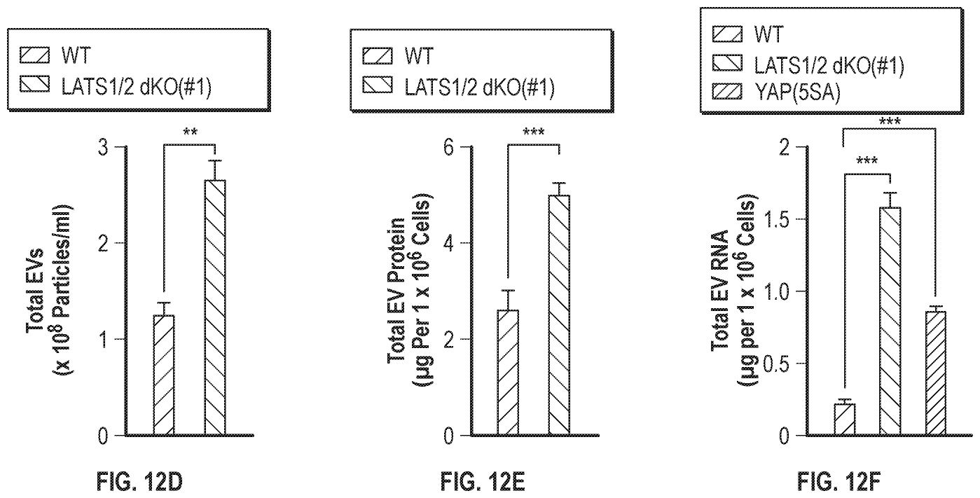

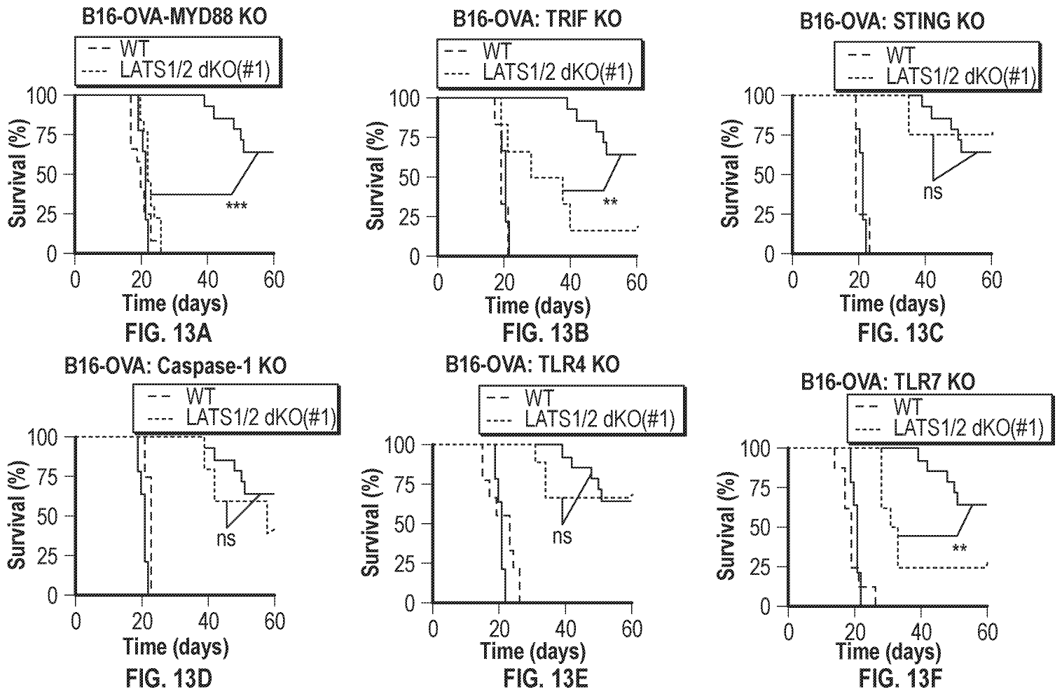

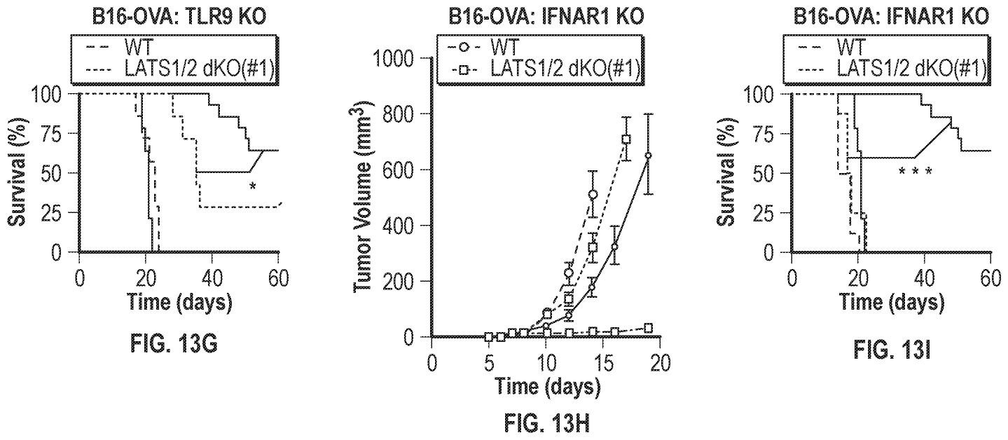

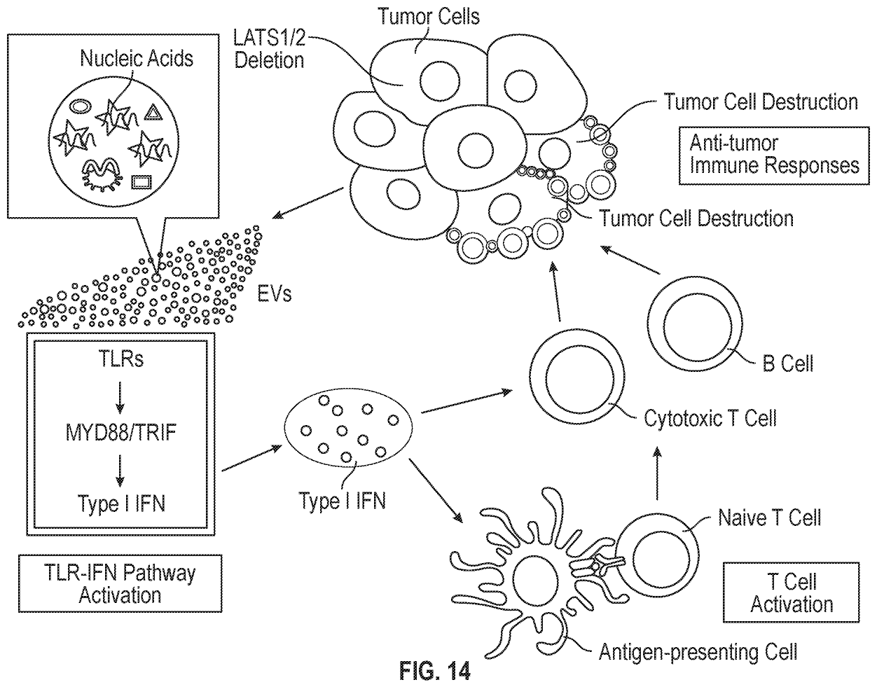

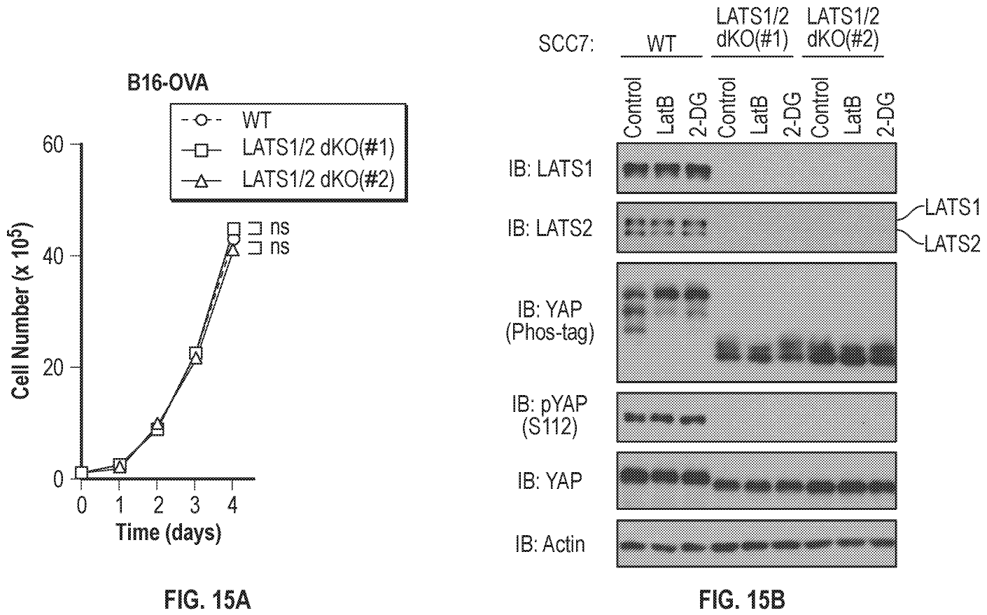

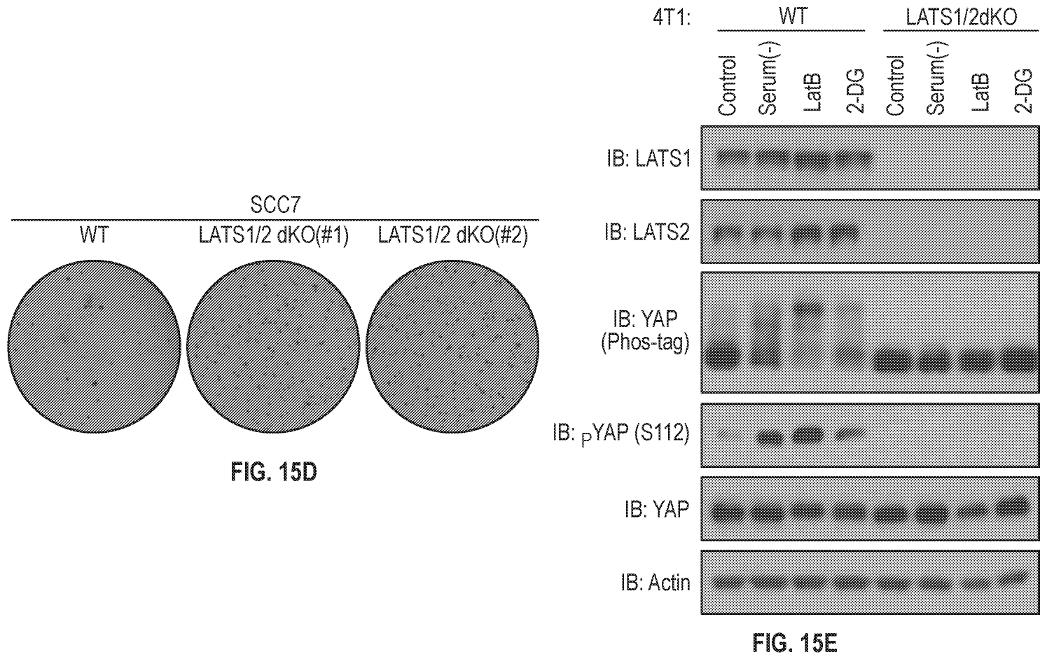

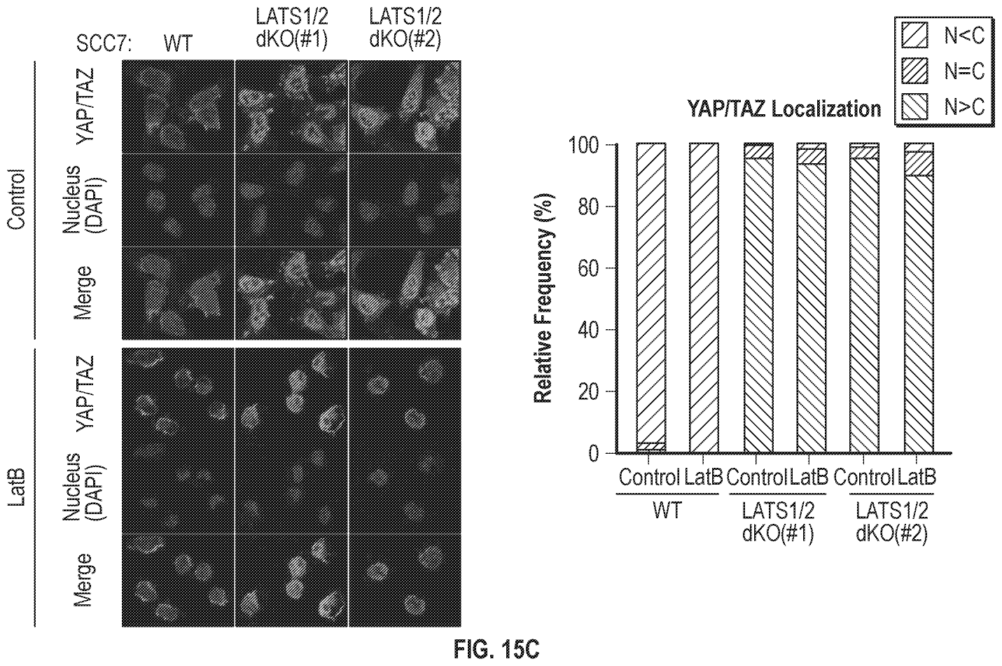

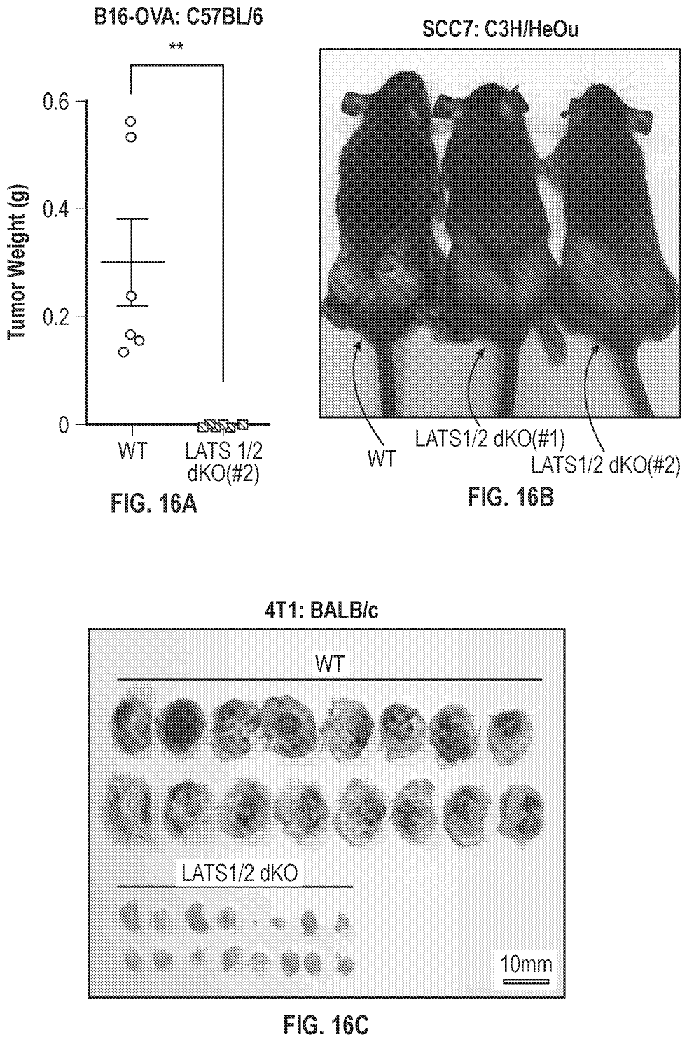

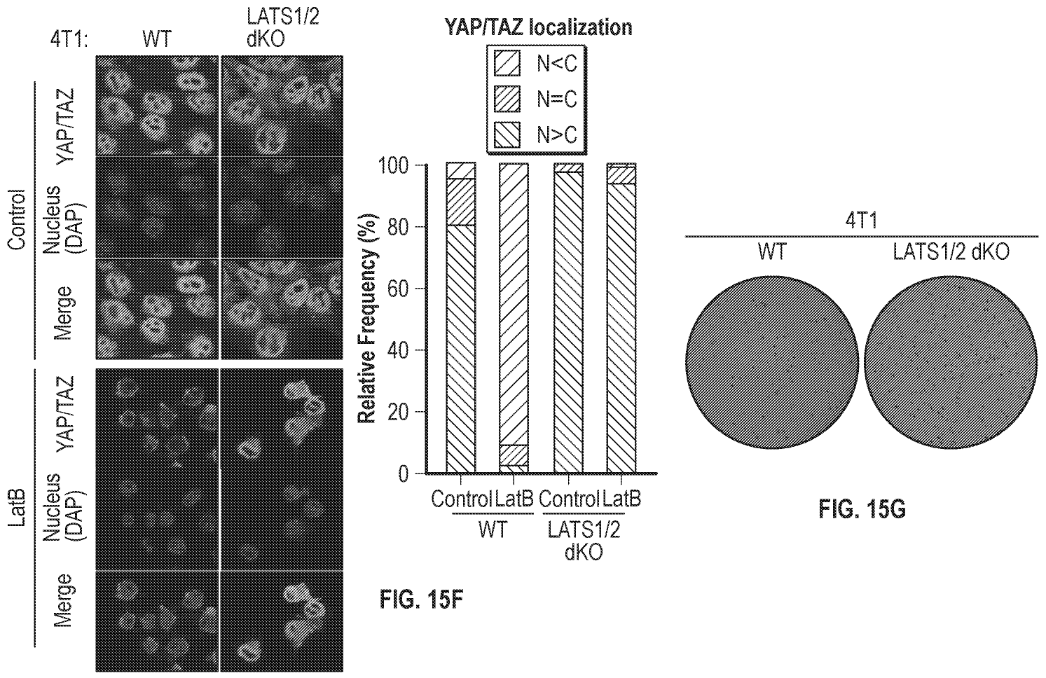

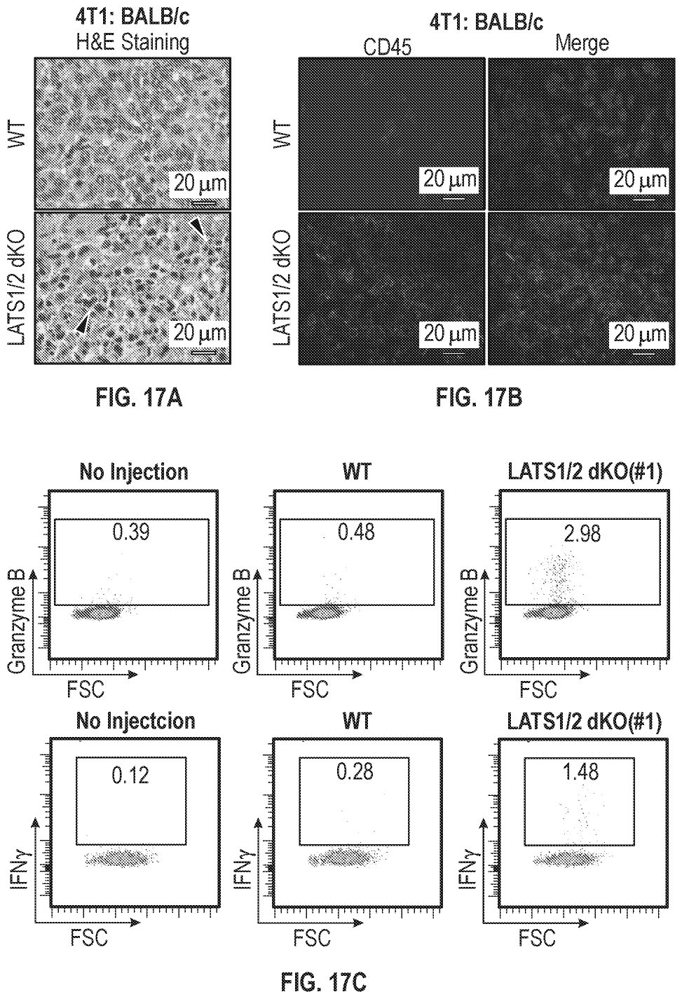

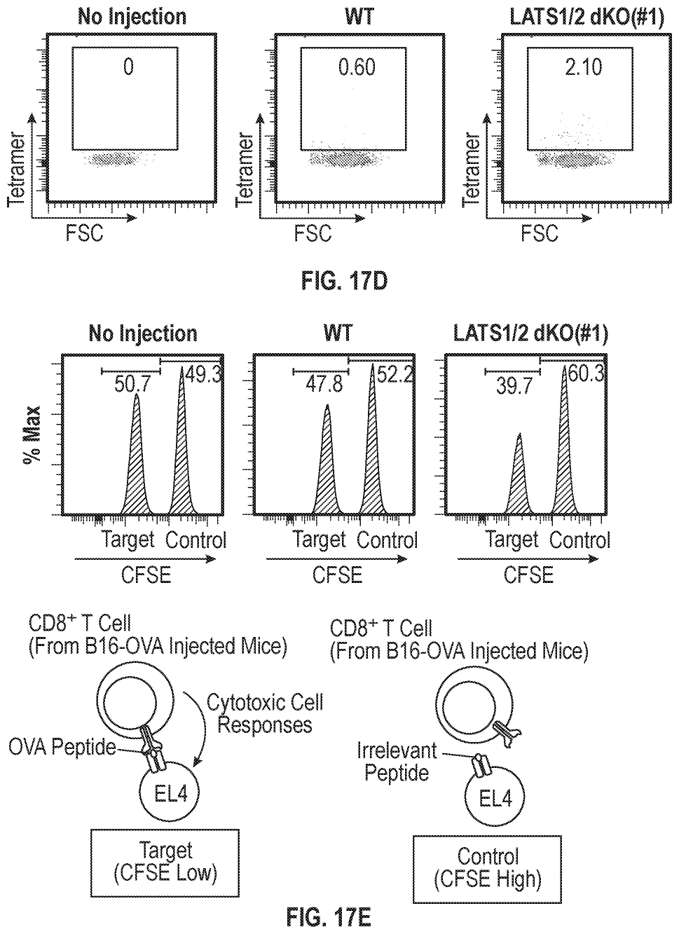

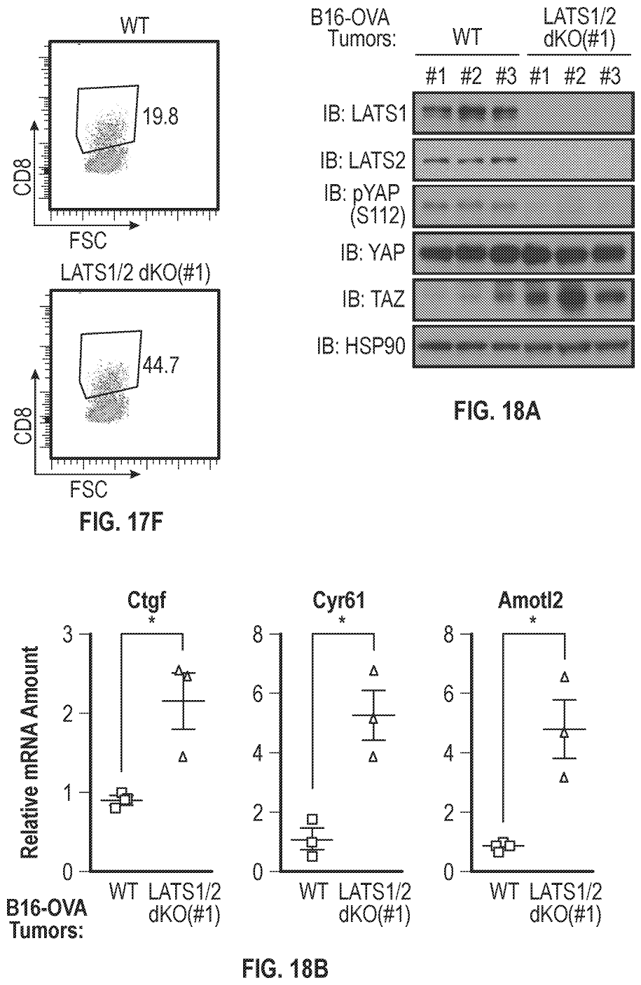

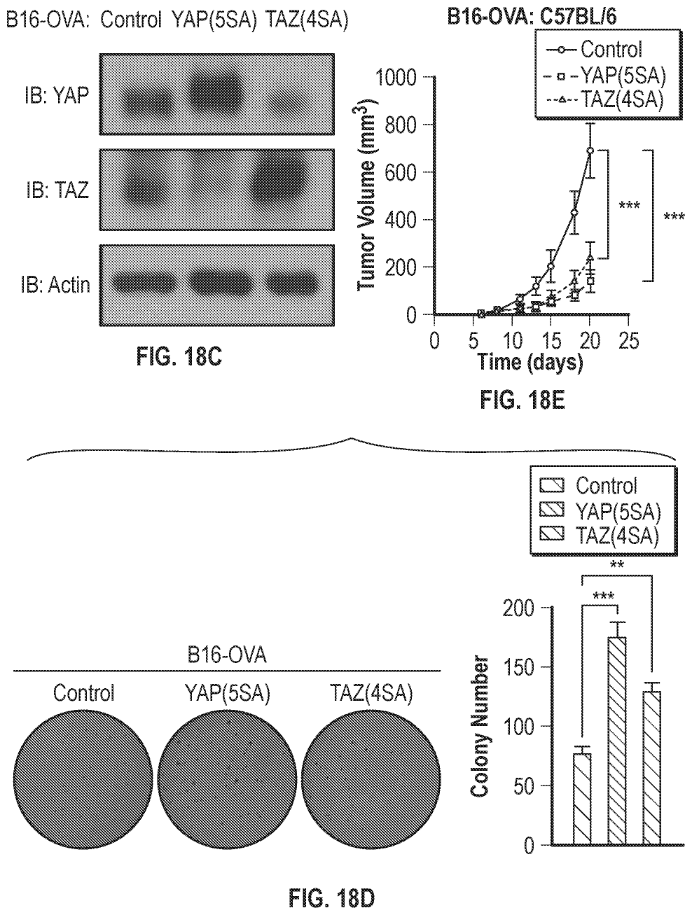

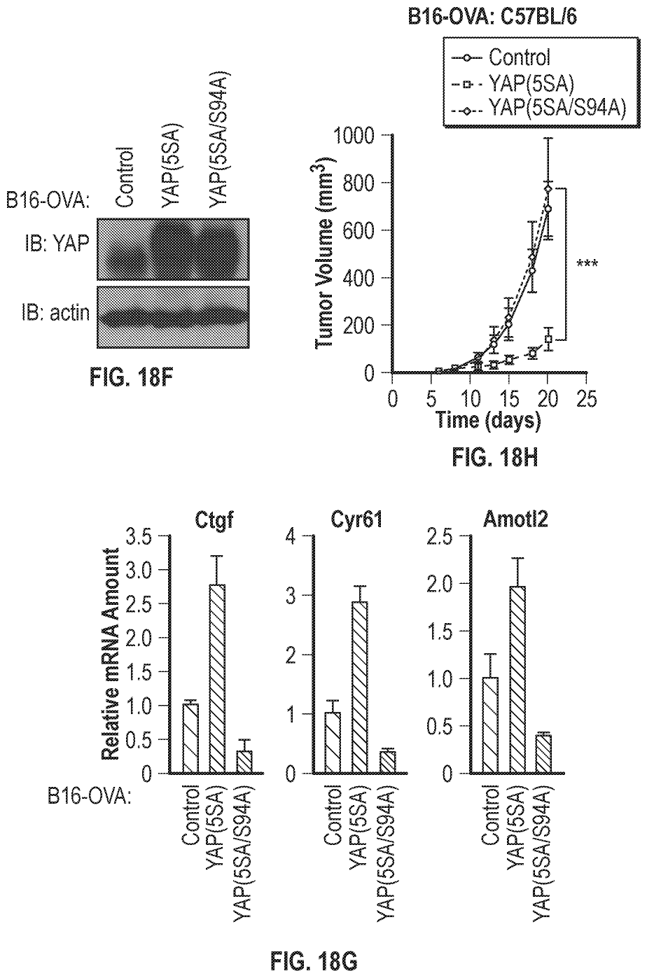

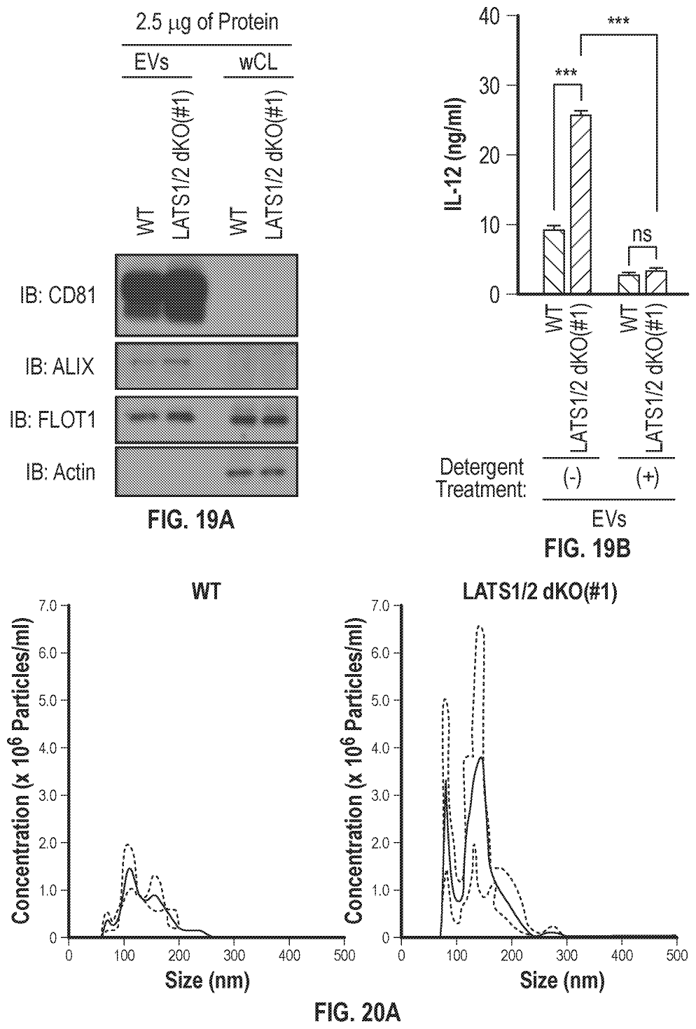

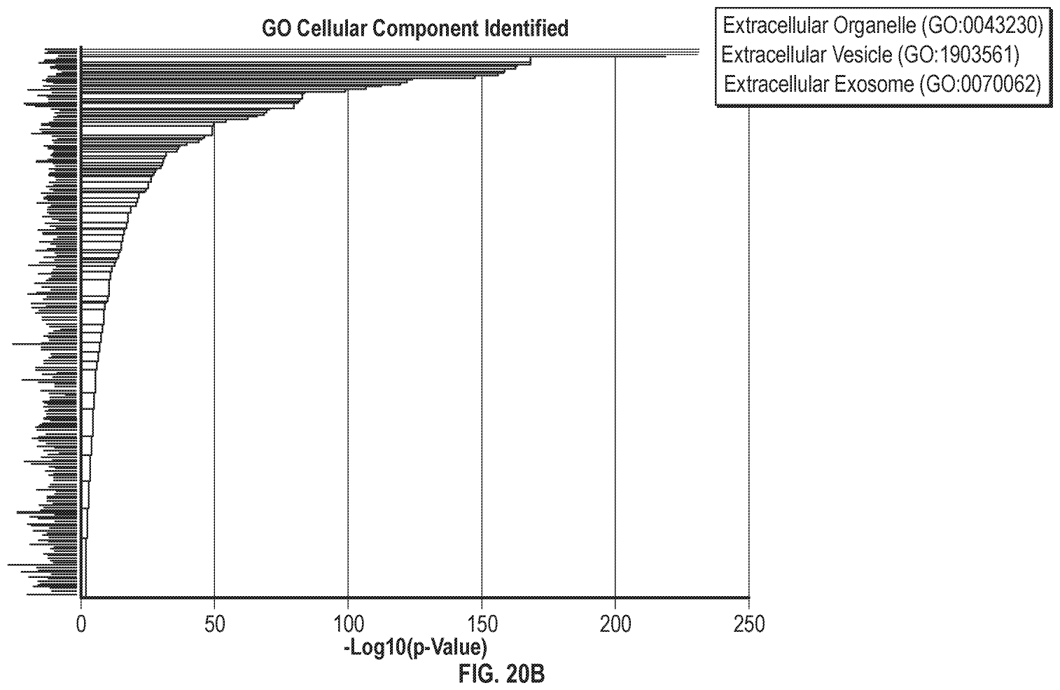

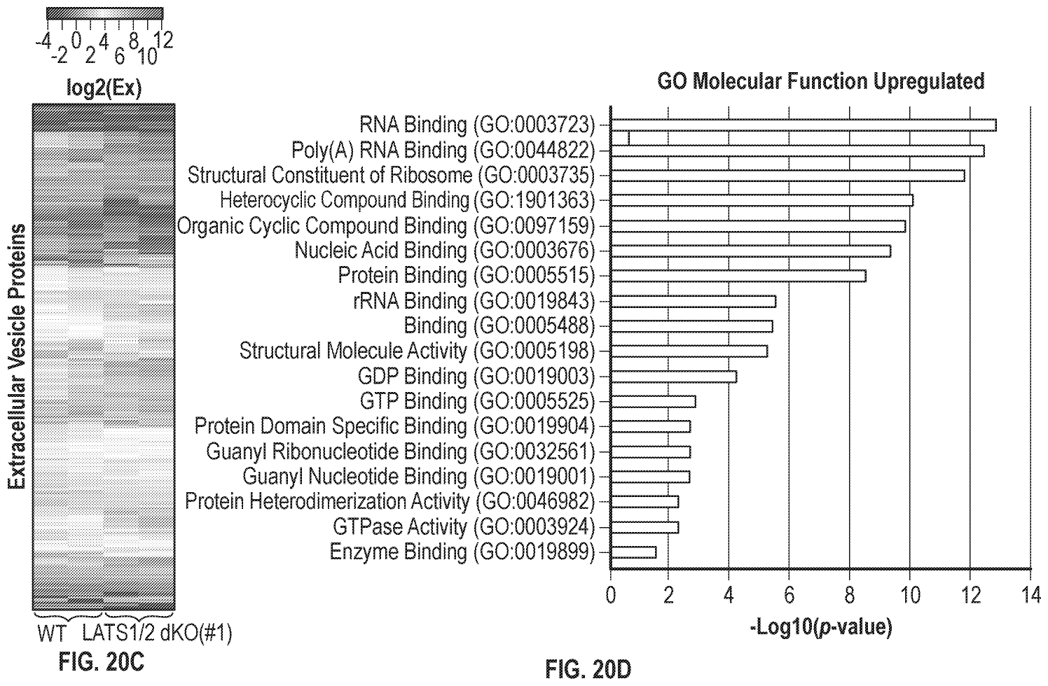

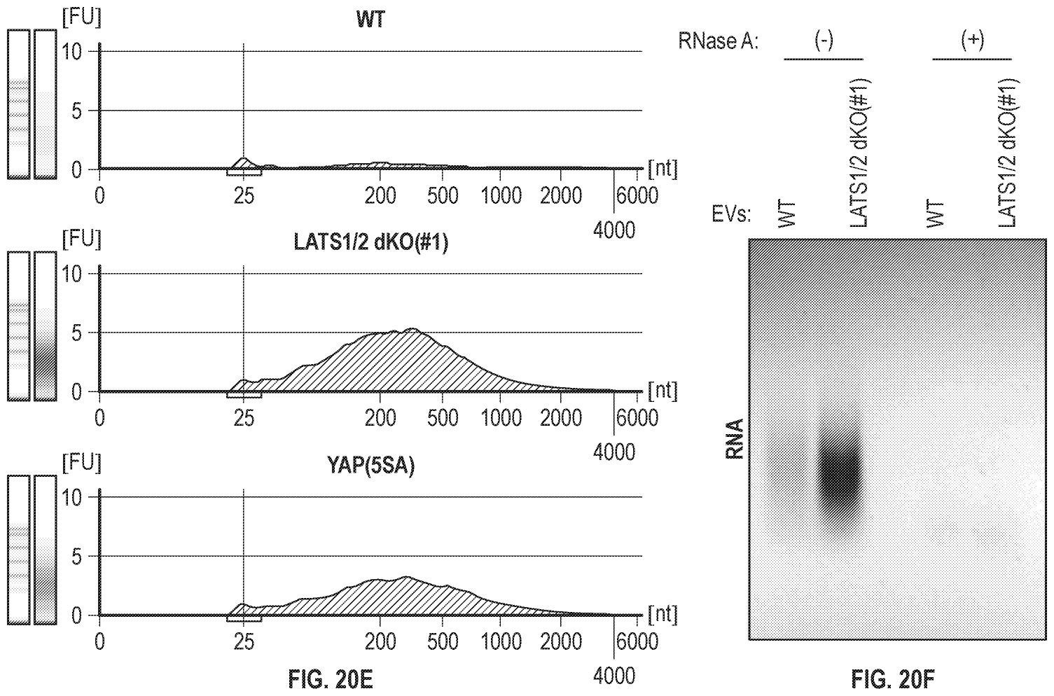

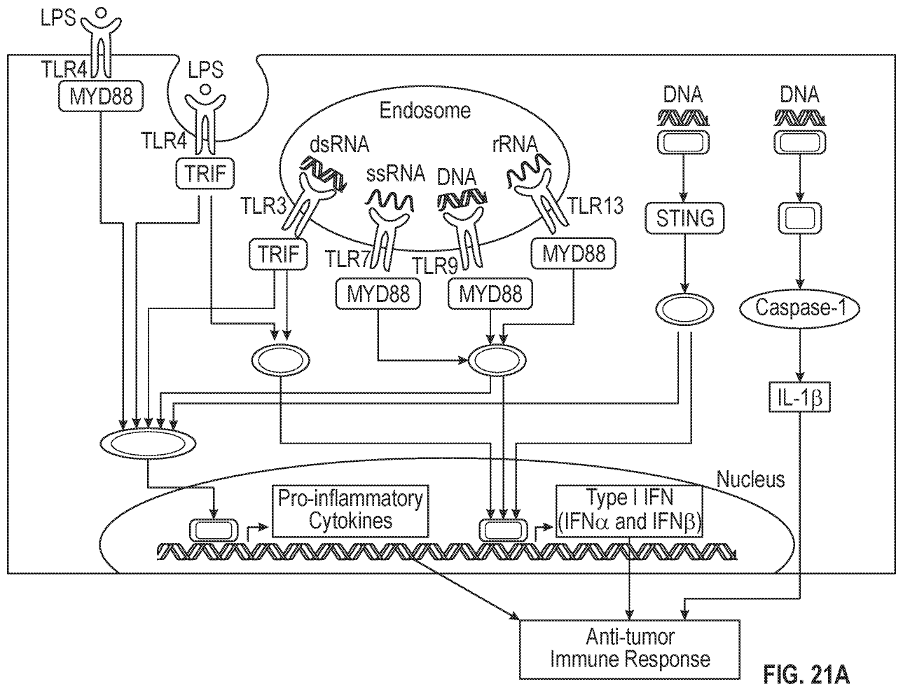

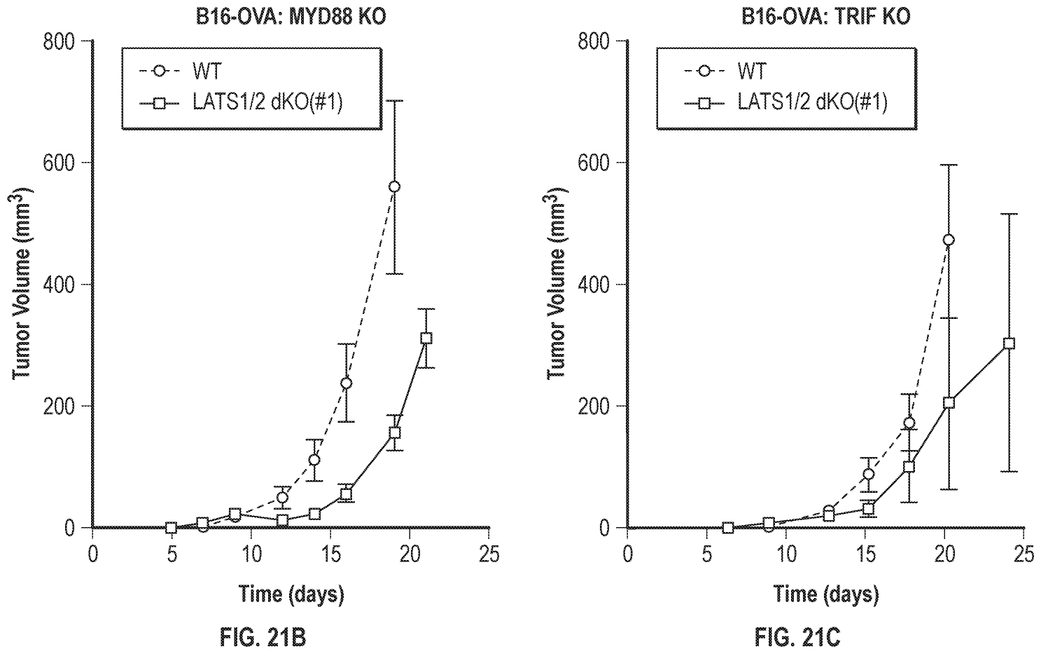

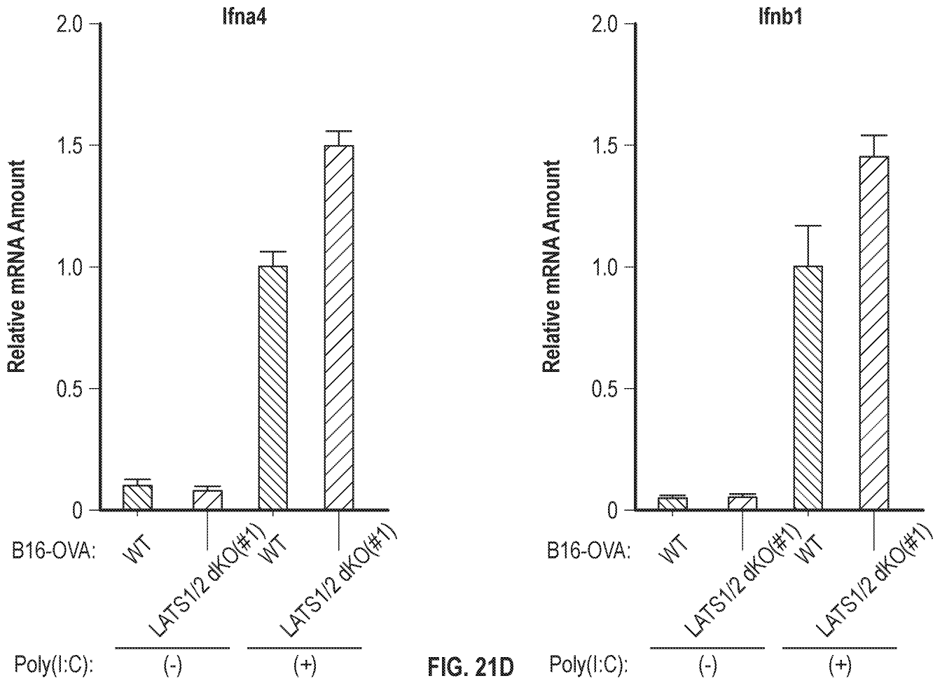

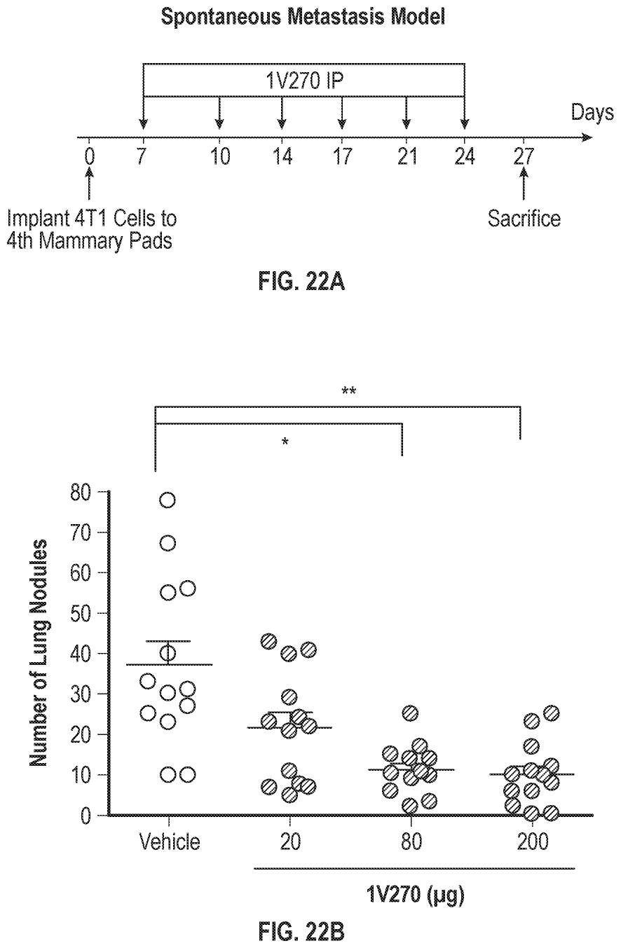

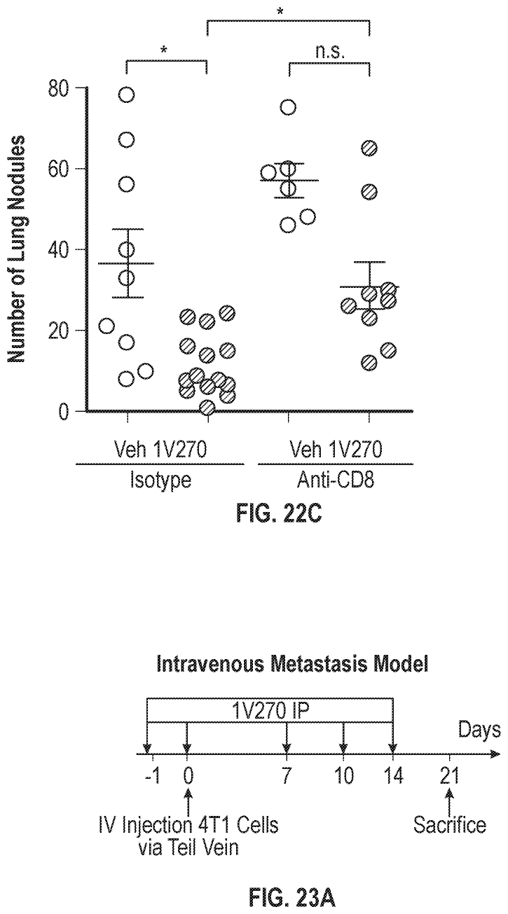

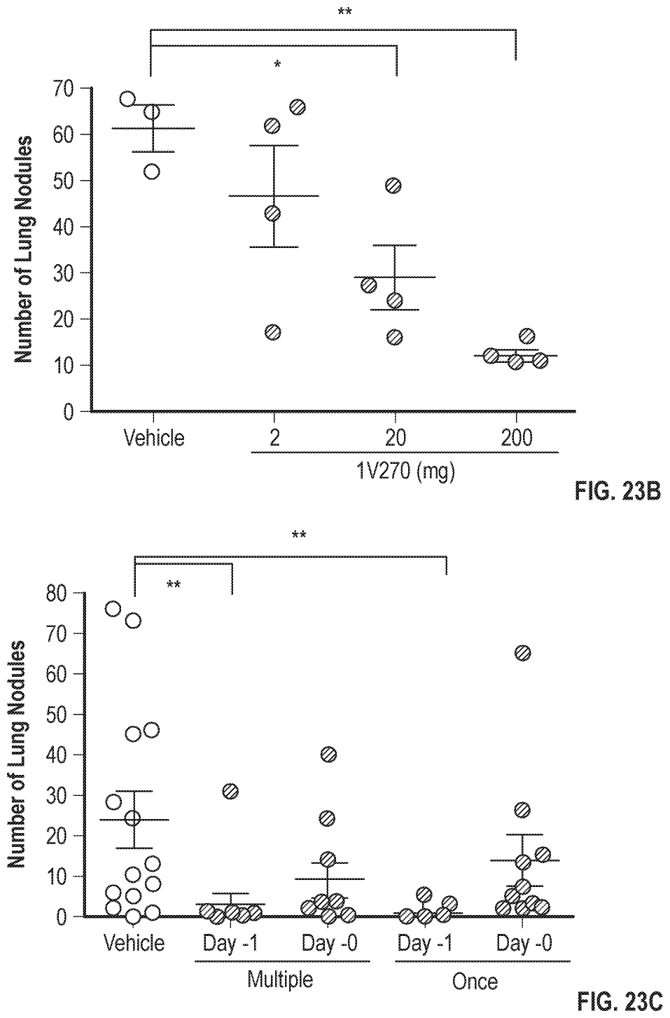

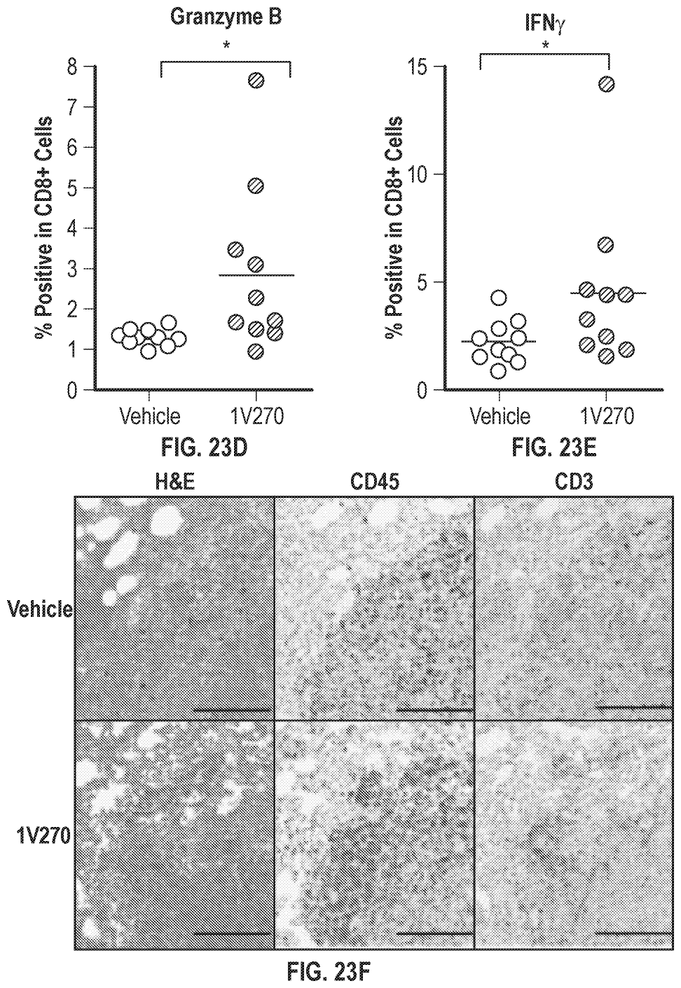

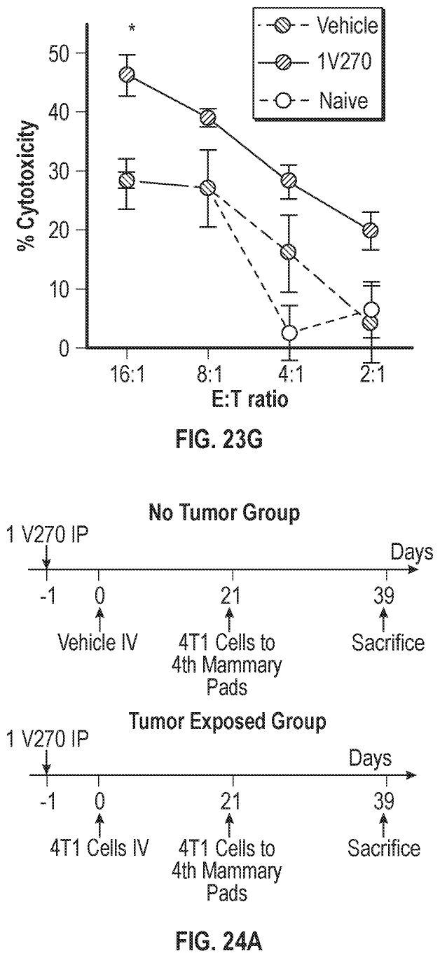

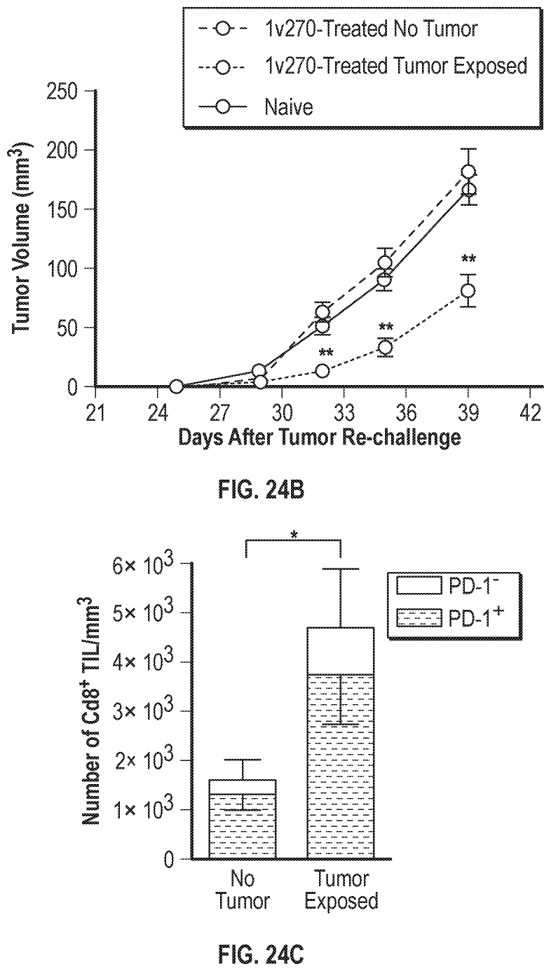

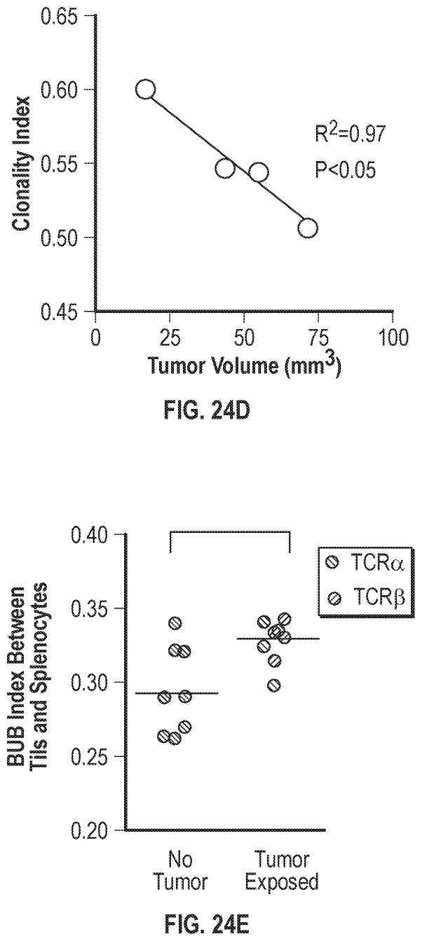

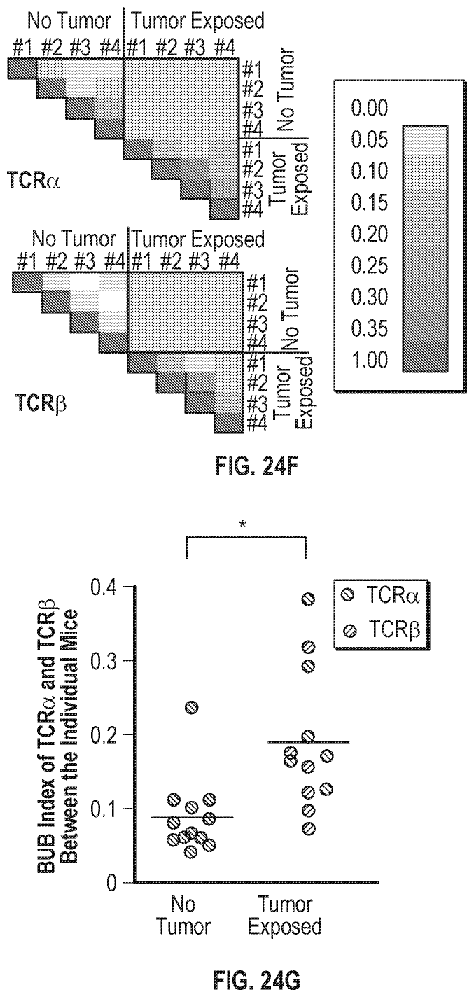

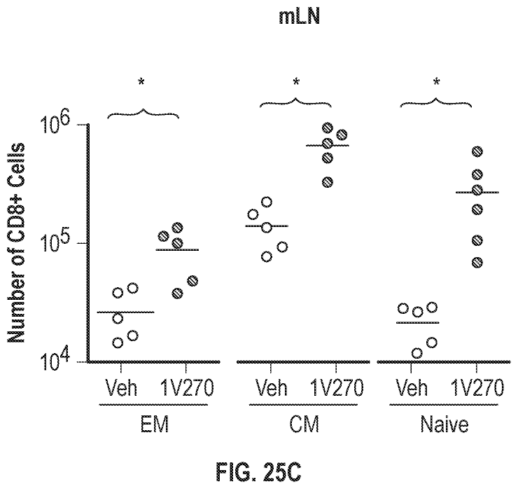

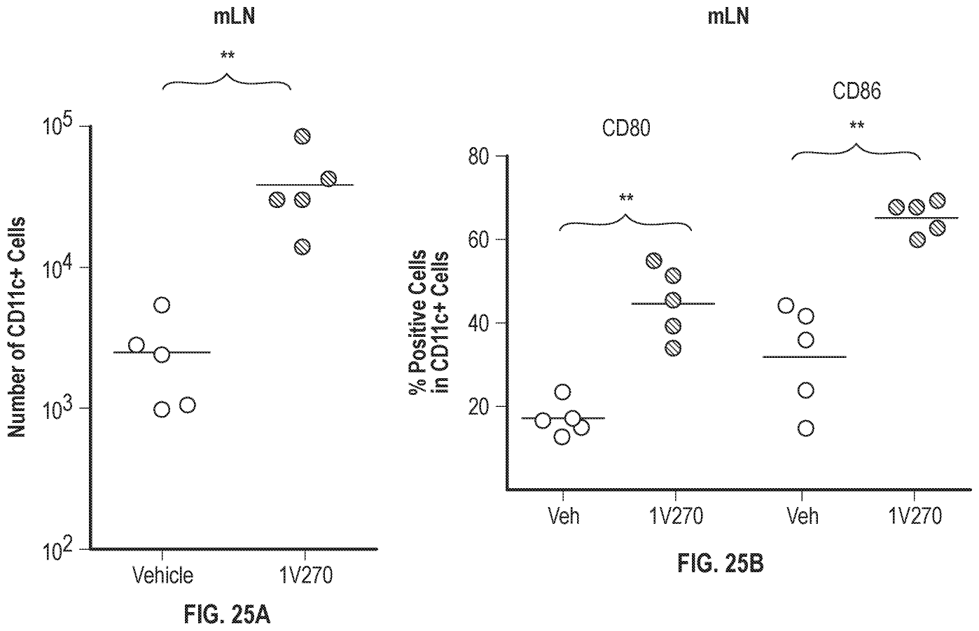

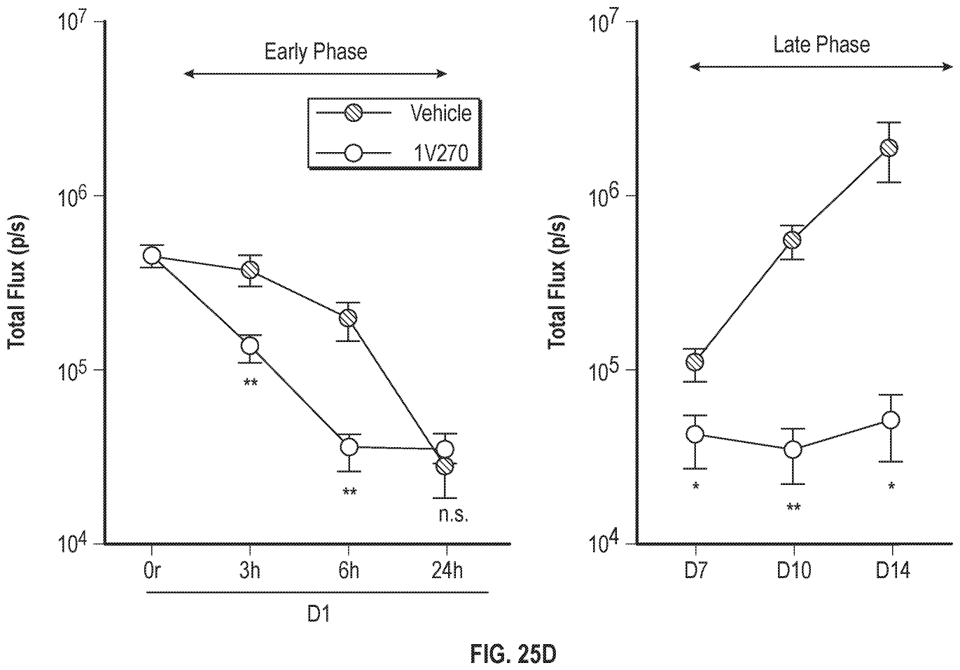

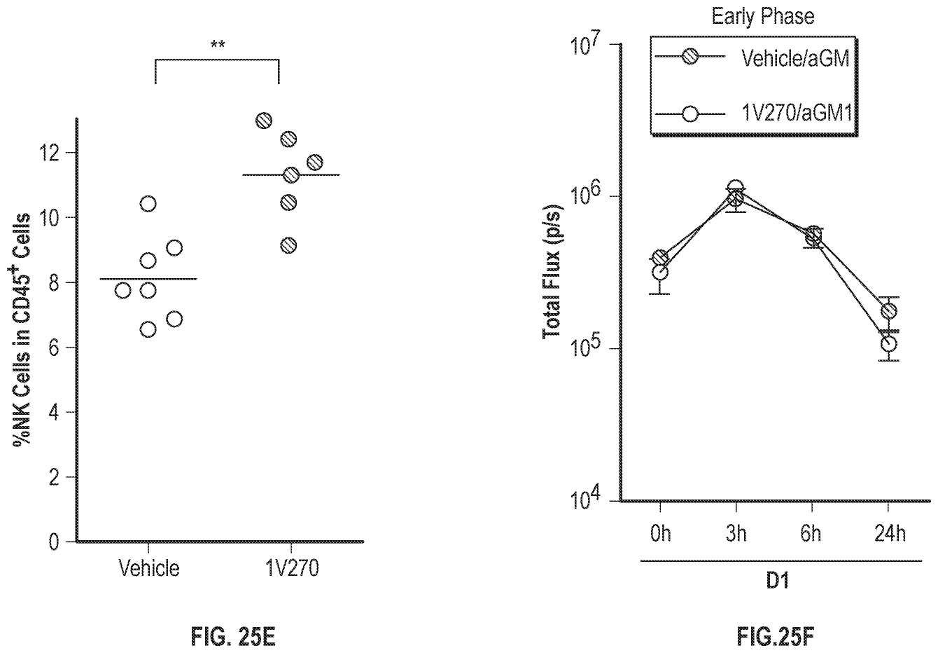

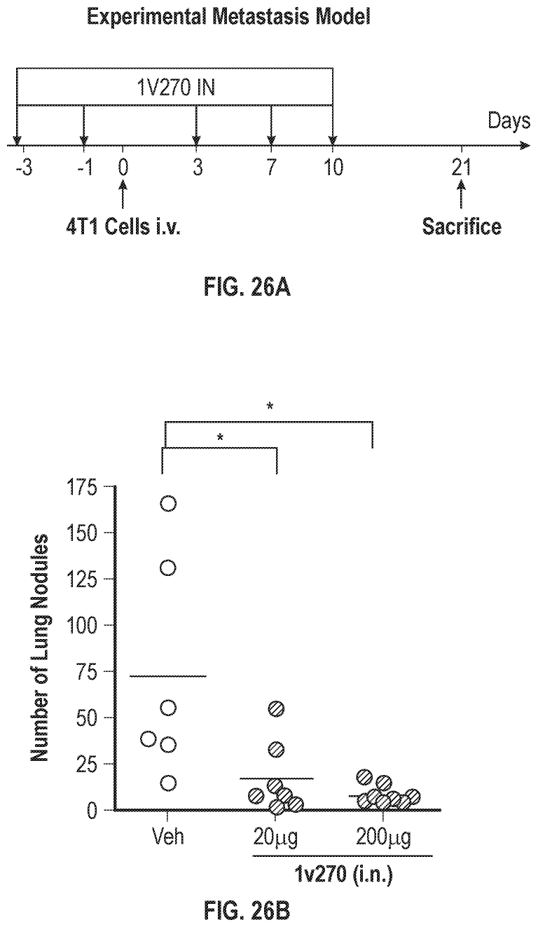

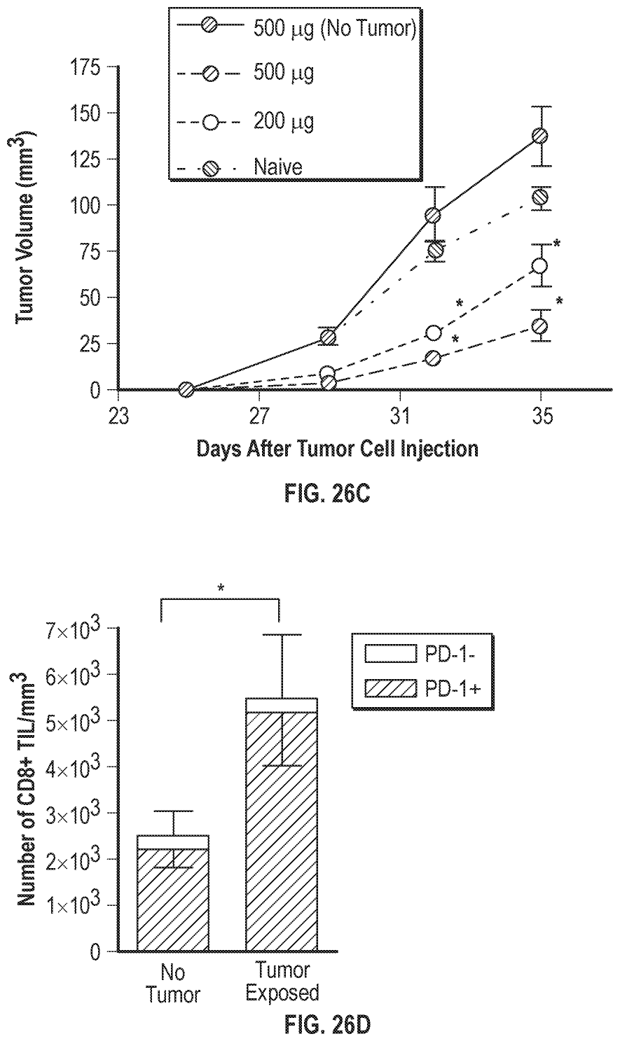

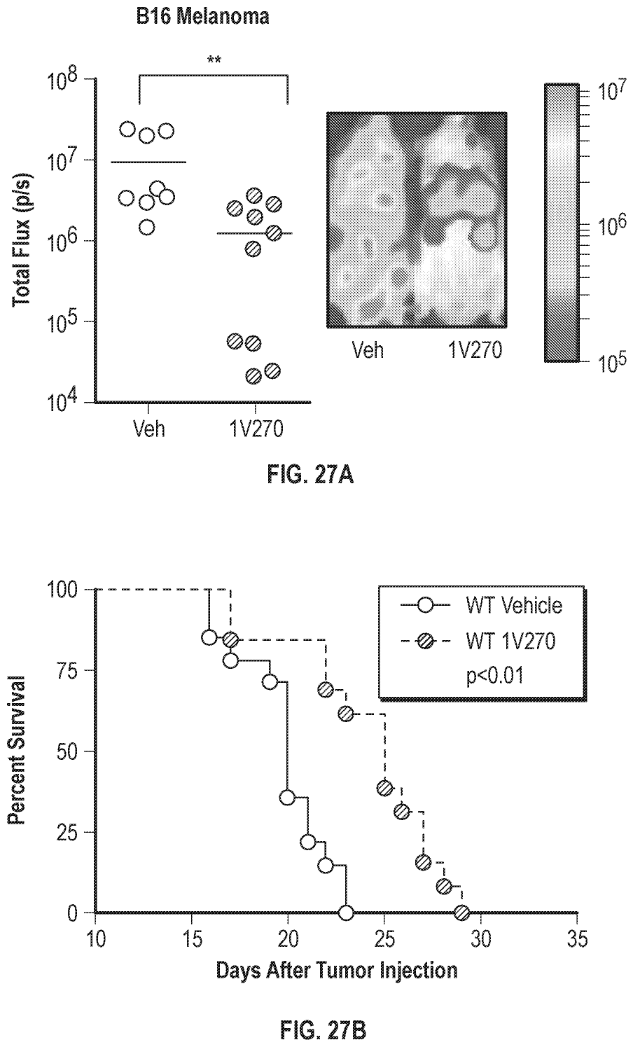

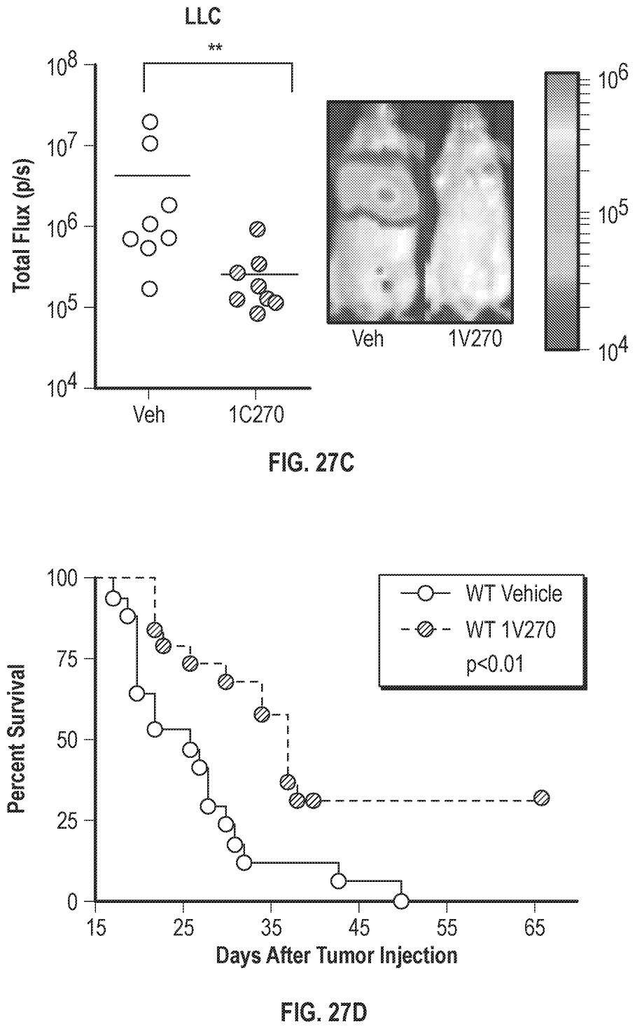

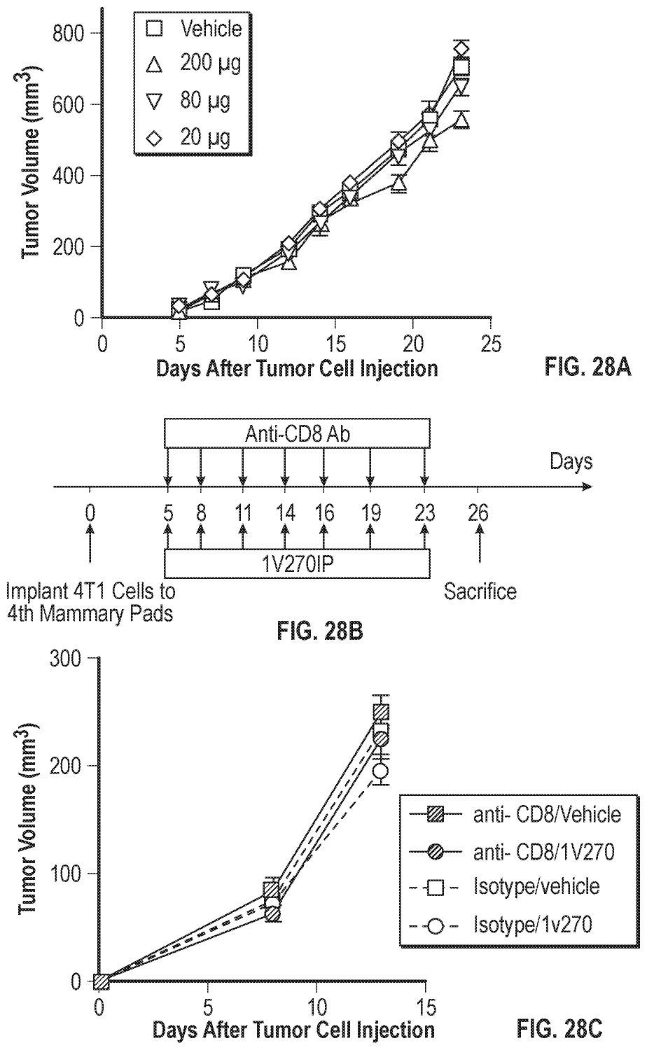

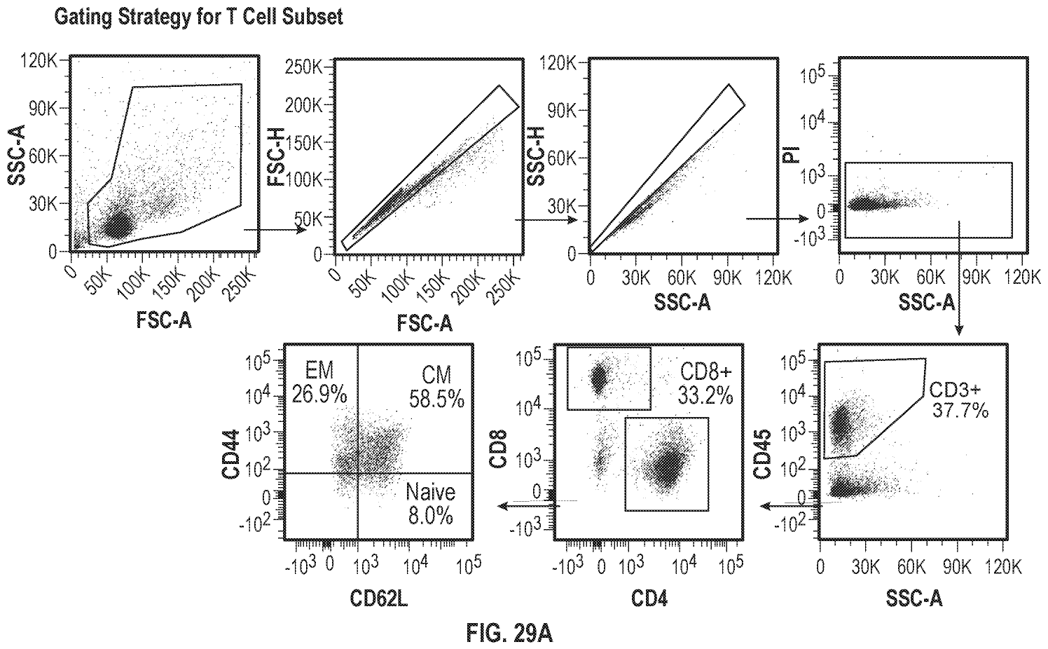

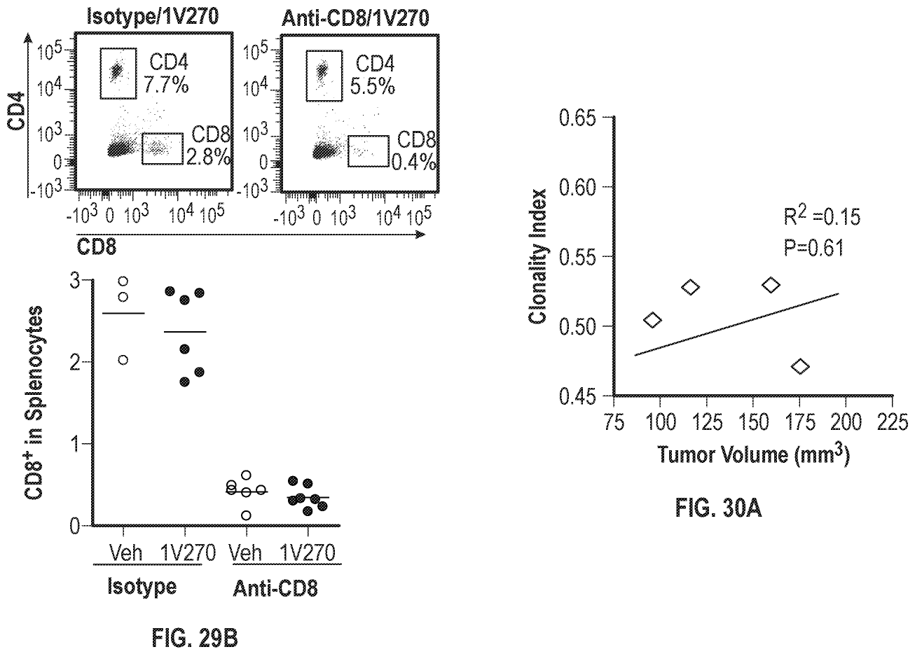

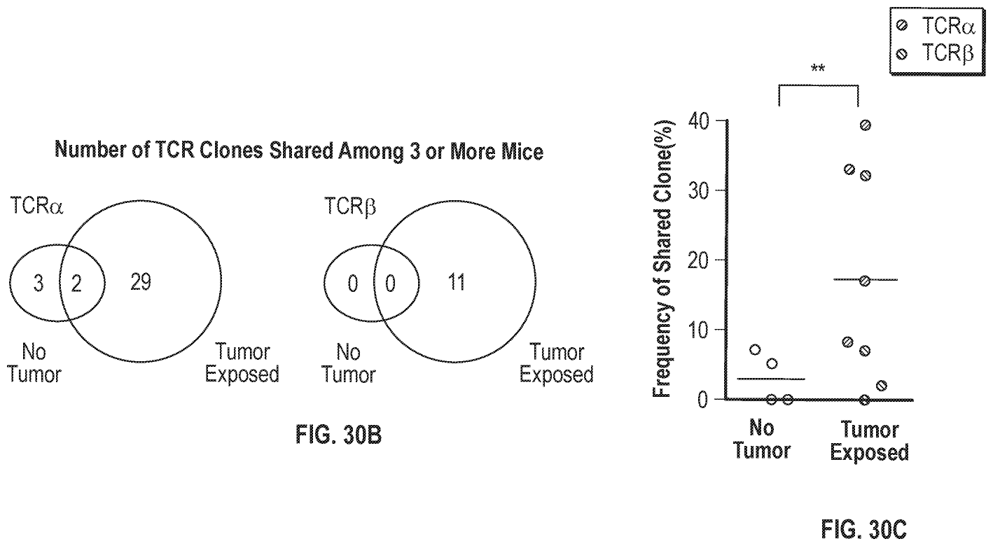

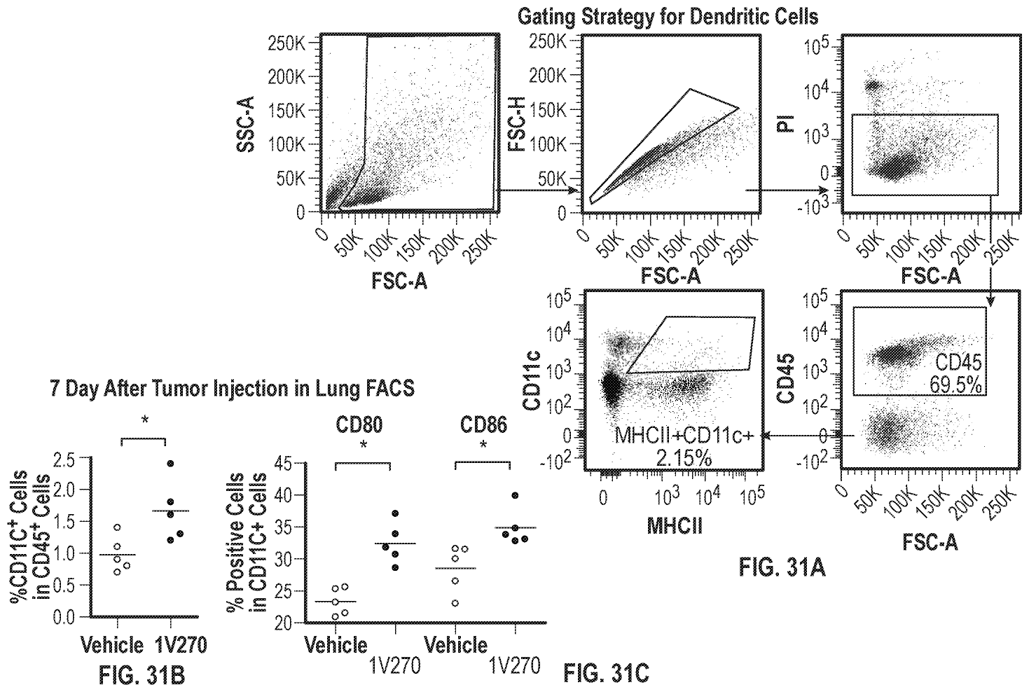

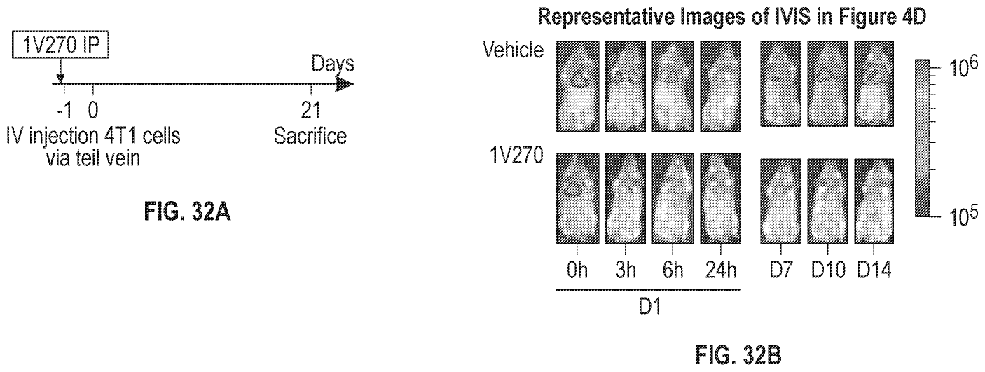

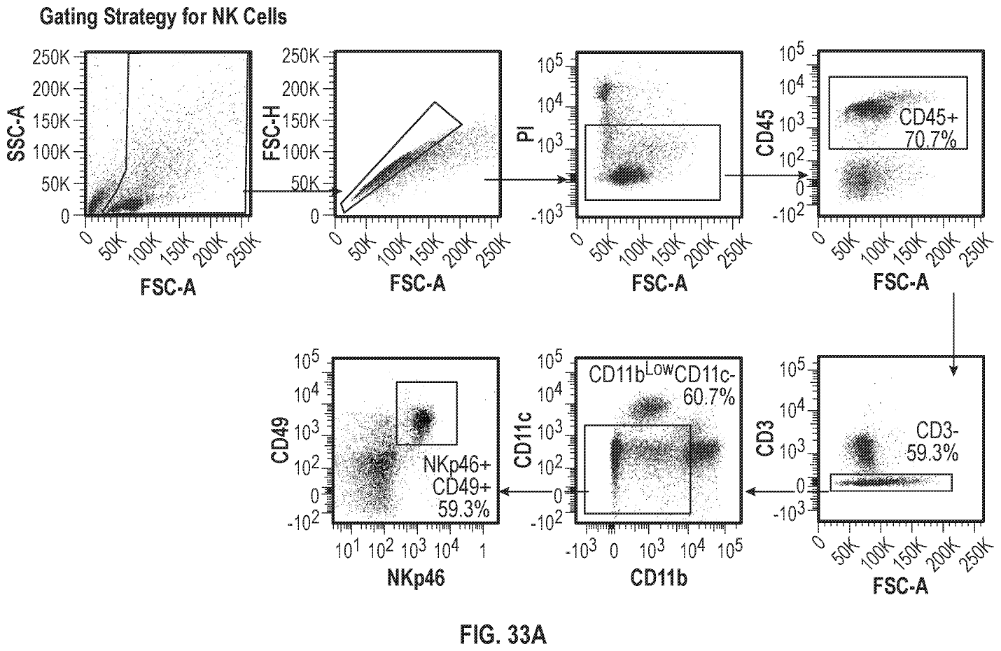

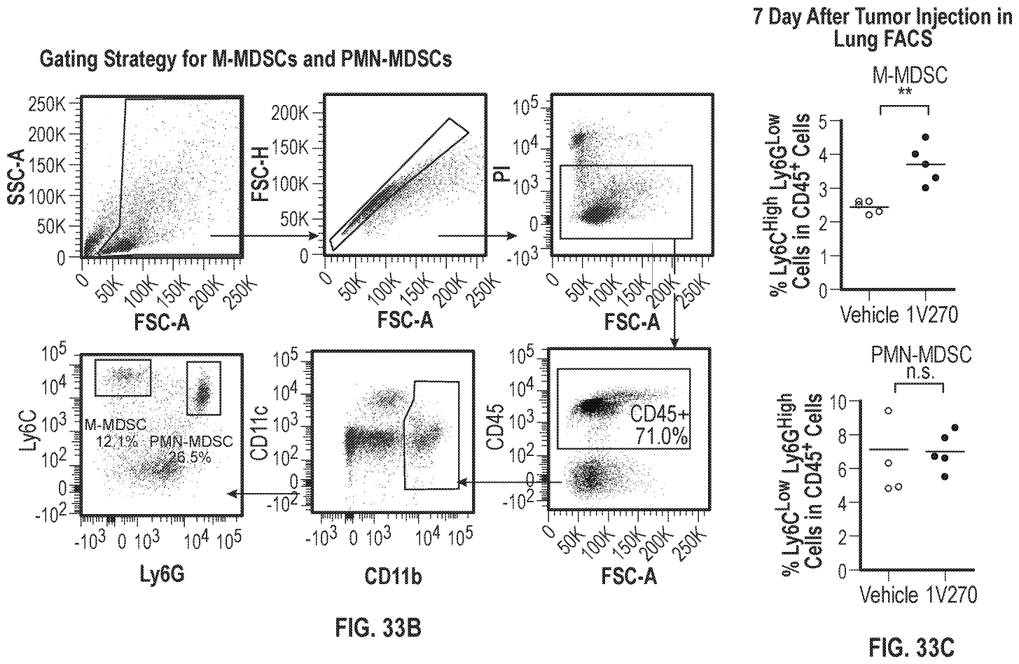

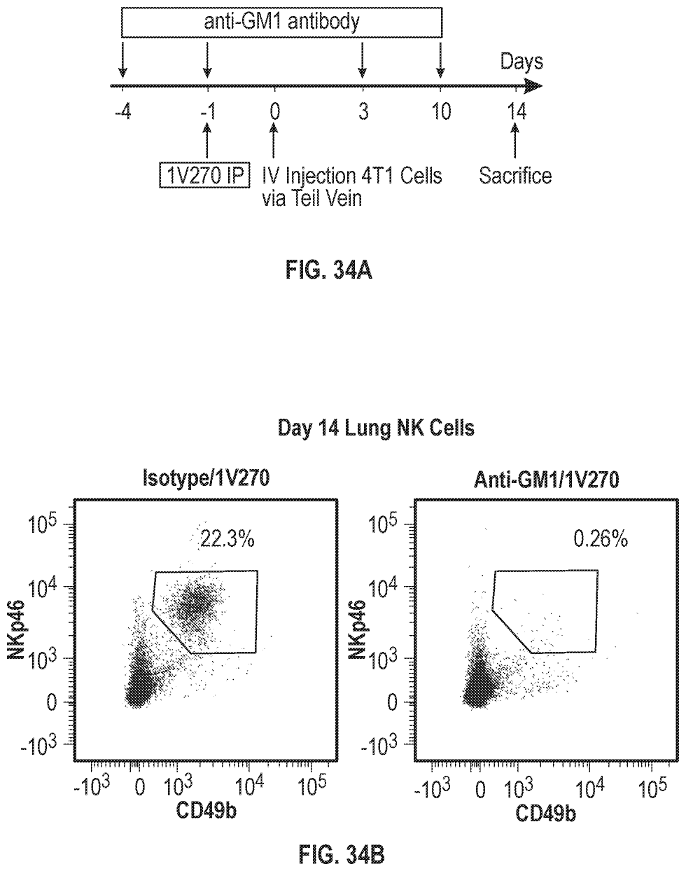

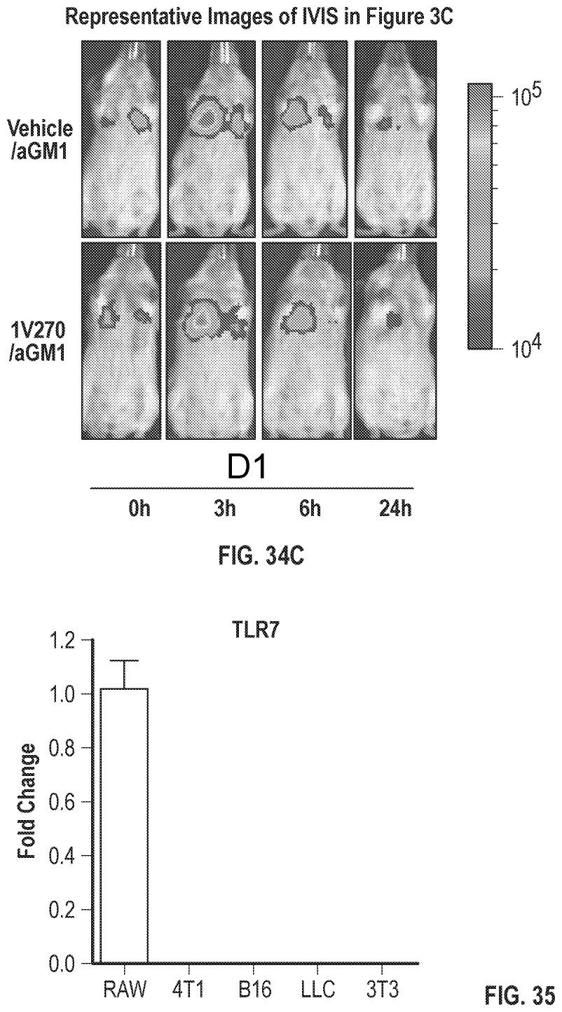

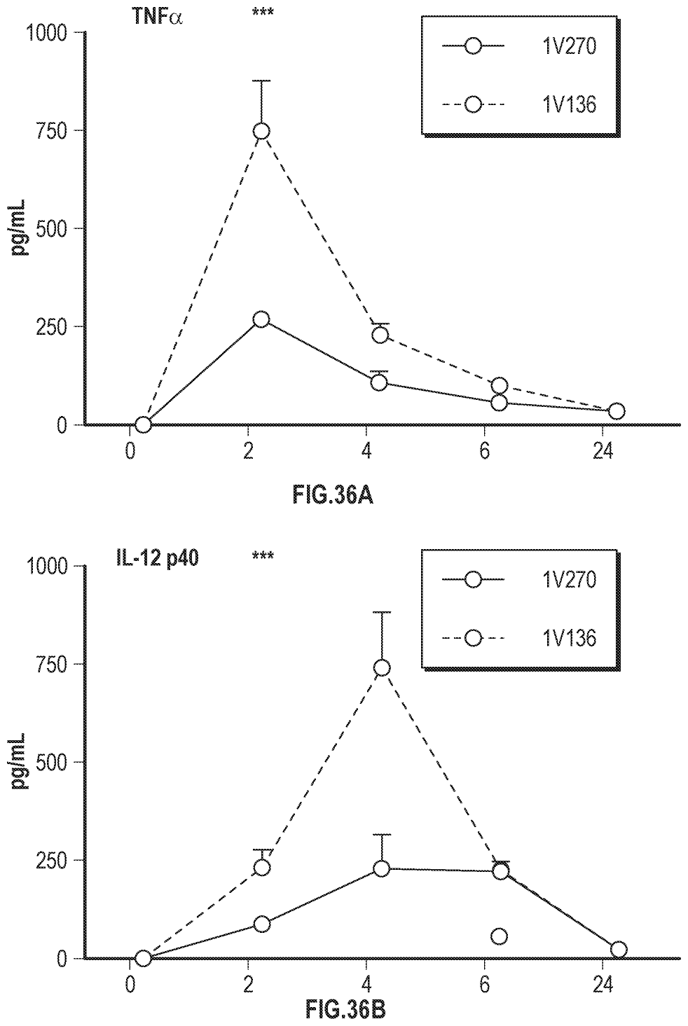

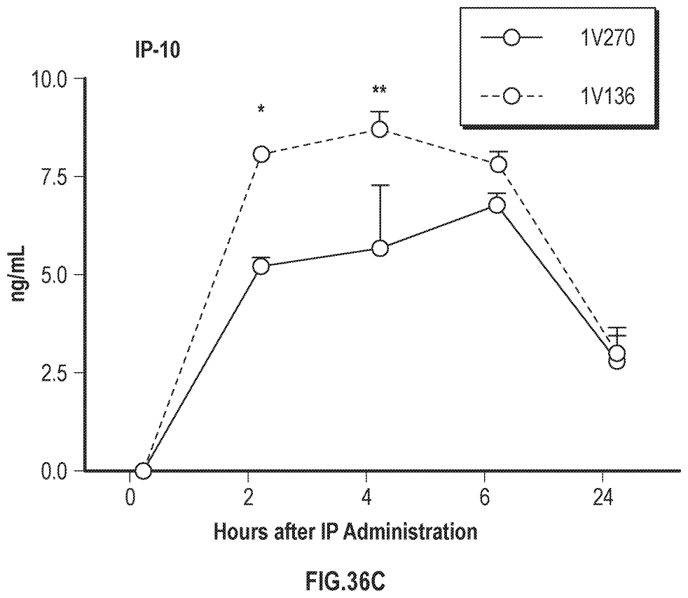

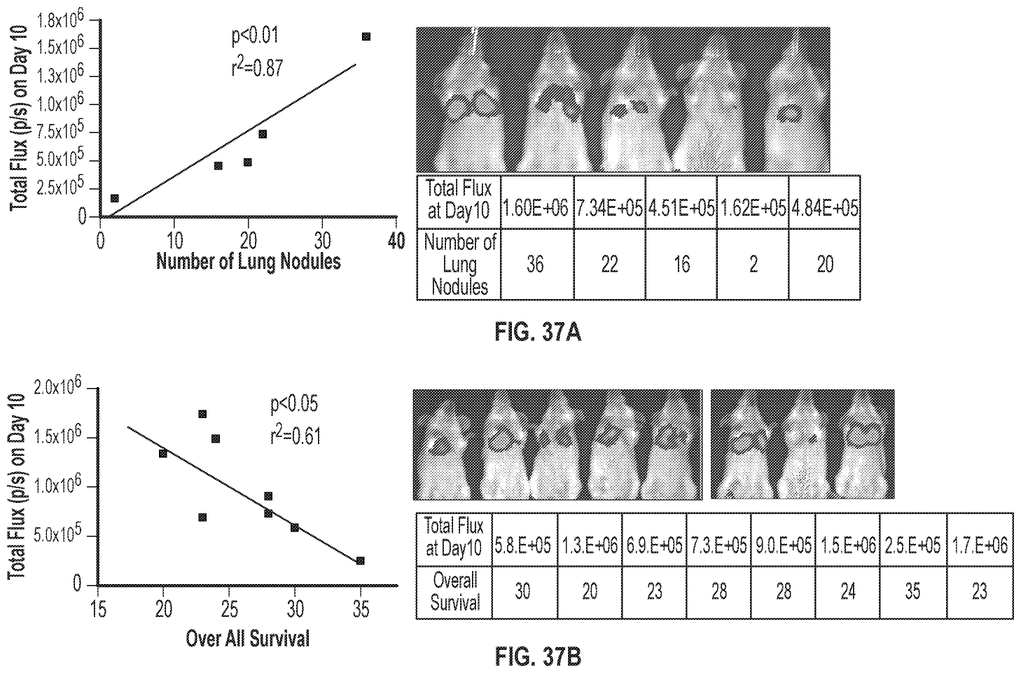

FIGS. 6A-6F. Extracellular vesicles released from LATS1/2 null cells are immunostimulatory. (A) Bone marrow-derived dendritic cells (BMDCs) were pretreated with conditioned medium from WT or LATS1/2 dKO B16-OVA melanoma cells (or control medium) and pulsed with OVA protein. BMDCs were then subjected to an in vitro cross-presentation assay using CFSE-labeled CD8⁺ T cells isolated from OVA-specific T cell receptor transgenic OT-I mice. OT-ICD8⁺ T cells proliferate when they were stimulated with OVA antigen via cross-presentation by BMDCs, resulting in dilution of CFSE content. Representative histograms of the gated CD8⁺ T cells are shown in the left panel. The division index was calculated and data are presented as means±SEM of 3 independent experiments in the right panel. ***p<0.001, one-way ANOVA test followed by Tukey's multiple comparison test. (B) BMDCs were stimulated with conditioned medium or EVs from WT or LATS1/2 dKO B16-OVA cell culture supernatants and then IL-12 levels in the culture supernatants were determined by ELISA. Data are represented as mean±SEM; n=3 independent experiments for conditioned medium stimulation, n=4 independent experiments for EVs stimulation. *p<0.05; ***p<0.001, one-way ANOVA test followed by Tukey's multiple comparison test. (C) C57BL/6 mice were inoculated with irradiated WT B16-OVA cells at the base of the tail and EVs freshly isolated from culture supernatants of equal numbers of WT or LATS1/2 dKO B16-OVA cells were injected every 3 days (day 0, 3, 6, and 9). At day 12, mice were challenged with WT B16-OVA melanoma and tumor growth was monitored. Data are represented as mean±SEM; n=8 tumors for each group. The tumor growth curves shown in FIG. 4B are presented in a lighter color for reference. p value was determined using two-way ANOVA test, comparing WT EV-immunized group [WT+WT EVs→WT] to LATS1/2 dKO EV-immunized group [WT+LATS1/2 dKO EVs→WT]. ***p<0.001. (D) EVs isolated from culture supernatants of equal numbers of WT or LATS1/2 dKO B16-OVA cells were subjected to nanoparticle tracking analysis (NanoSight) to quantify the number and size distribution. The numbers of particles are presented as means±SEM of 3 independent experiments. **p<0.01, unpaired t-test. (E) Protein concentrations of EVs isolated as in (D) were determined. Data are means±SEM of 6 independent experiments. ***p<0.001, unpaired t-test. (F) EVs were isolated from culture supernatants of equal numbers of WT, LATS1/2 dKO, or YAP(5SA)-overexpressing B16-OVA cells and RNA concentrations were determined by Agilent TapeStation. Data are means±SEM of 3 independent experiments. ***p<0.001, one-way ANOVA test followed by Tukey's multiple comparison test.

FIG. 7. Exemplary protocol for collection and expansion of autologous T cell therapy.

FIGS. 8A-8E. LATS1/2 Deletion Enhances Anchorage-Independent Tumor Cell Growth In Vitro (A) Wild-type (WT) and two independent clones of LATS1/2 double knockout (dKO) B16-OVA melanoma cells were serum starved or treated with Latrunculin B (LatB) and then subjected to immunoblot (IB) analysis with antibodies to the indicated proteins. (B) LatB-treated or non-treated (control) B16-OVA cells were subjected to immunostaining analysis. YAP/TAZ subcellular localization was determined by immunofluorescence staining for endogenous YAP/TAZ (green) along with DAPI for DNA (blue). Representative images are presented in the left panel. (right panel) Cells in five randomly selected views (about 100 cells) were selected for the quantification of YAP/TAZ localization. N, nuclear; C, cytoplasmic. (C) B16-OVA cells were subjected to soft-agar colony-formation assay, and the colonies were stained with crystal violet for quantification. (D) Soft-agar colony-formation assay of SCC7 squamous cell carcinoma cells. E) Soft-agar colony-formation assay of 4T1 breast cancer cells. Data are presented as means±SD from three independent experiments (C-E). The p values were determined using a one-way ANOVA test followed by Tukey's multiple comparison test (C and D) or an unpaired t test (E). **p<0.01; ***p<0.001. See also FIG. 51.

FIGS. 9A-9G. Loss of LATS1/2 in Tumors Inhibits Tumor Growth In Vivo (A) Equal numbers of WT or LATS1/2 dKO B16-OVA cells were transplanted into C57BL/6 mice, and tumor growth was monitored after the indicated times. Data are presented as means±SEM; n=8 tumors for each group. ***p<0.001, two-way ANOVA test. (B) C57BL/6 mice were injected with WT or LATS1/2 dKO B16-OVA melanoma, and tumor weight was determined 20 days after transplantation. Data are presented as means±SEM; n=6 tumors for WT, and n=8 tumors for LATS1/2 dKO (note that two of the tumors were completely rejected). **p<0.01, Mann-Whitney test. (C) Kaplan-Meier tumor-free survival curves for mice injected with WT or LATS1/2 dKO B16-OVA cells are shown (n=14 mice for each group). ***p<0.001, log rank test. (D) WT or two independent clones of LATS1/2 dKO SCC7 cells were transplanted into C3H/HeOu mice, and tumor growth was monitored after the indicated times. Data are presented as means±SEM; n=8 tumors for each group. The p values were determined using a two-way ANOVA test, comparing each group to the WT group. ***p<0.001. (E) Kaplan-Meier tumor-free survival curves for mice injected with WT or LATS1/2 dKO SCC7 cells are shown (n=4 mice for each group). The p values were determined using a log-rank test, comparing each group to the WT group. **p<0.01. F and G) In (F), BALB/c mice were injected with WT or LATS1/2 dKO 4T1 cells, and primary tumor weight was determined 28 days after transplantation. (G) WT or LATS1/2 dKO 4T1 cells were transplanted into the mammary fat pad of BALB/c mice, and lung metastasis of the primary tumor was determined 28 days after transplantation. Normal lung tissue was stained with black India ink, whereas tumor nodules remained white. The gross appearance of the lungs (left panel) and the tumor nodules on the lungs (right panel) were examined. Data are presented as means±SEM; n=16 tumors in (F), and n=8 mice in (G) for each group. ***p<0.001, Mann-Whitney test. See also FIG. 16.

FIGS. 10A-10I. LATS1/2 Deficiency in Tumor Cells Induces Host Anti-tumor Immunity (A) WT or LATS1/2 dKO B16-OVA melanoma cells were injected into C57BL/6 mice. Tumors were paraffin embedded and stained with H&E 12 days after transplantation. Arrowheads indicate infiltration of inflammatory cells. (B) Frozen sections from WT or LATS1/2 dKO B16-OVA melanomas were subjected to immunostaining analysis of CD45 (red) along with DAPI for DNA (blue). (C) WT and two independent clones of LATS1/2 dKO B16-OVA melanoma cells were subjected to immunoblot (IB) analysis with antibodies to the indicated proteins. (D-F) In (D), C57BL/6 mice were injected (or not injected) with WT or LATS1/2 dKO B16-OVA melanoma cells, and serum anti-OVA IgG concentrations were determined by ELISA 12 days after transplantation. (E) Splenocytes from C57BL/6 mice injected as in (D) were re-stimulated ex vivo with SIINFEKL pep tide and then subjected to flow-cytometric analysis. SIINFEKL is an OVA-derived pep tide being presented through the major histocompatibility complex class I (MHC class I) molecule H-2Kb. Frequency of CD8⁺ T cells expressing activation markers, Granzyme B or interferon g (IFNg), was determined. (F) Splenocytes from C57BL/6 mice injected as in (D) were subjected to flow-cytometric analysis. OVA-specific CD8⁺ T cells were quantified using Kb-SIINFEKL tetramers and plotted as a percentage of total CD8⁺ T cells. Data are presented as means±SEM; n=4 mice for the uninjected group, n=10 mice for the WT-injected group, and n=10 mice for the LATS1/2 dKO-injected group. ***p<0.001, one-way ANOVA test followed by Tukey's multiple comparison test. (G) C57BL/6 mice were injected as in (D), and the inguinal lymph nodes were cultured ex vivo with OVA protein. IFNg levels in the culture supernatants were determined by ELISA. Data are presented as means±SEM of triplicate cultures of pooled lymph node cells from 4 mice per group. ***p<0.001, one-way ANOVA test followed by Tukey's multiple comparison test. (H) C57BL/6 mice were injected as in (D) and CD8⁺ T cells were isolated from splenocytes. T cell cytotoxicity assay was performed with CFSE (carboxyfluorescein succinimidyl ester)-labeled EL4 cells ex vivo and the percentage of specific killing was plotted. Data are presented as means±SEM of five independent experiments with pooled CD8⁺ T cells from 3-4 mice per group. ***p<0.001, one-way ANOVA test followed by Tukey's multiple comparison test. (I) WT or LATS1/2 dKO B16-OVA melanoma cells were injected into C57BL/6 mice, and tumors were subjected to flow-cytometric analysis 12 days after transplantation. Data are presented as means±SEM of the percentage of CD8⁺ T cells infiltrating into tumors among total CD45⁺ cells; n=4 tumors for each group. ***p<0.001, unpaired t test. See also FIG. 17.

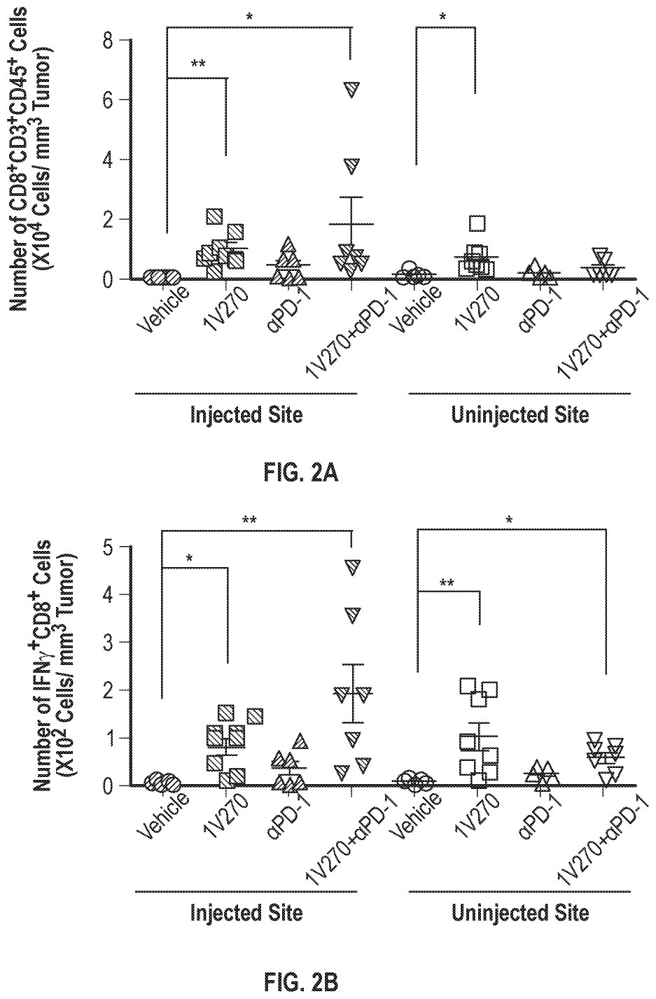

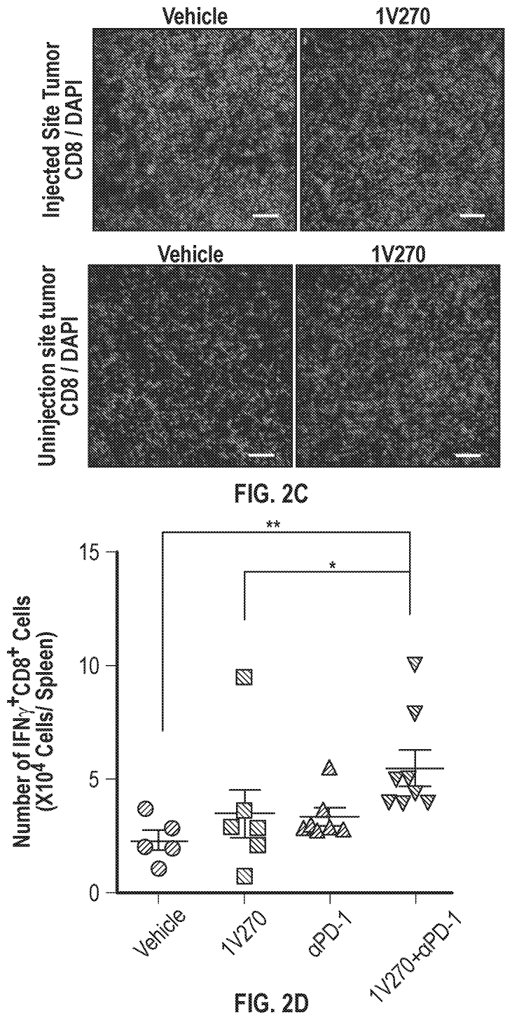

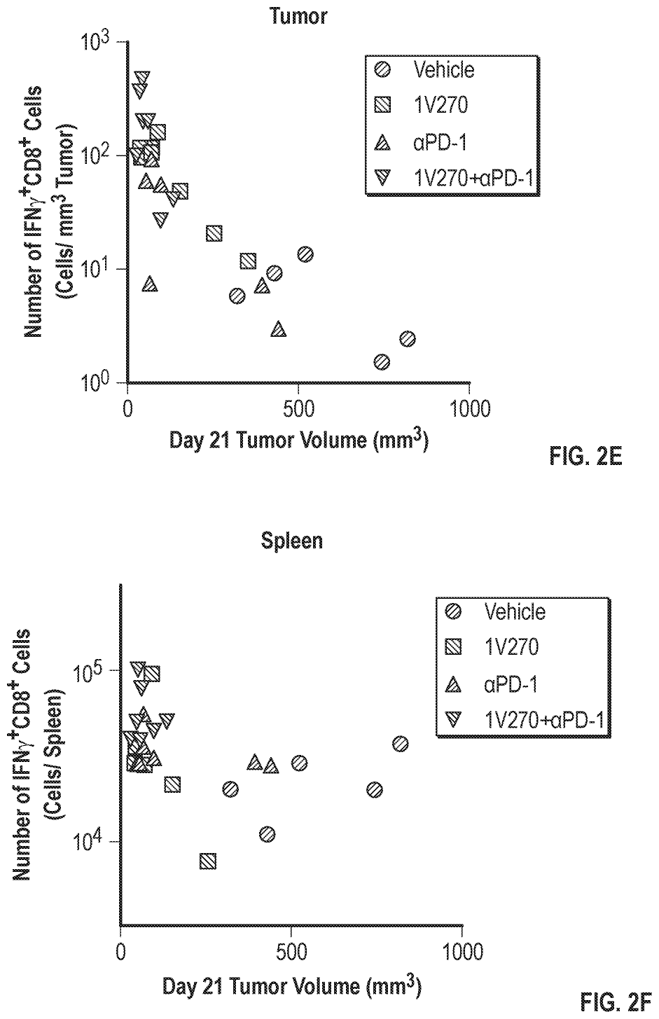

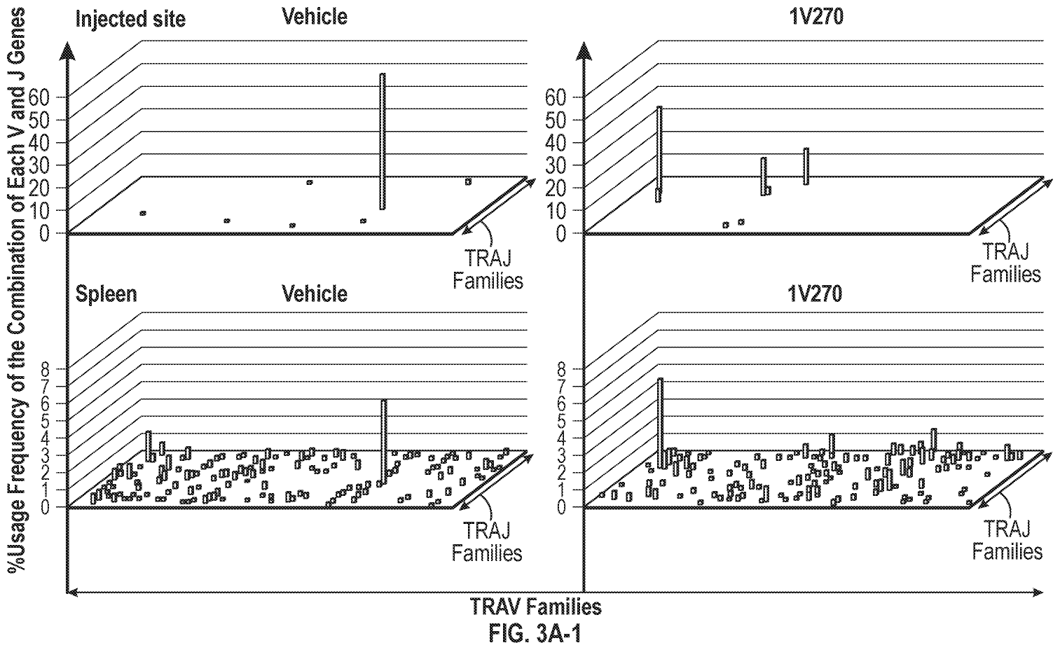

FIGS. 11A-11G. LATS1/2 Deletion in Tumors Stimulates Host Adaptive Immunity and Enhances Tumor Vaccine Efficacy (A) WT or LATS1/2 dKO B16-OVA melanoma cells were injected into C57BL/6 mice, and tumor growth was monitored after the indicated times (left panel). For coinjection experiments, WT and LATS1/2 dKO cells were injected into opposite flanks in the same mouse (right panel). “WT [with LATS1/2 dKO(#1)]” (blue line) indicates WT tumor growth, and “LATS1/2 dKO(#1) [with WT]” (yellow line) indicates LATS1/2 dKO tumor growth, in the co-injected mice. Data are presented as means±SEM; n=8 tumors for WT or LATS1/2 dKO group, n=6 tumors for each co-injected group. The p value was determined using two-way ANOVA test, comparing the “WT [with LATS1/2 dKO(#1)]” group to the WT group. ***p<0.001. (B and C) In (B), C57BL/6 mice were immunized intradermally at the base of the tail with equal numbers of irradiated WT or LATS1/2 dKO B16-OVA cells (or PBS control). 12 days after immunization, mice were challenged with WT B16-OVA melanoma, and tumor growth was monitored after the indicated times. Data are presented as means±SEM; n=8 tumors for each group. (C) Kaplan-Meier tumor-free survival curves for mice immunized and challenged as in (B) are shown (n=12 mice for each group). The survival curve of C57BL/6 mice challenged with WT B16-OVA melanoma without vaccination in FIG. 16C is also shown in light gray for reference. A schematic representation of vaccination experiment with irradiated-tumor cells is shown in the lower panel. The p value was determined using a two-way ANOVA test (B) or a log-rank test (C), comparing the WT-immunized group [WT/WT] to the LATS1/2 dKO-immunized group [LATS1/2 dKO(#1)/WT]. ***p<0.001. (D) C3H/HeOu mice were first injected with non-irradiated LATS1/2 dKO SCC7 cells. 60 days after the initial injection, mice designated tumor-free were rechallenged with WT SCC7 cells, and tumor growth was monitored [LATS1/2 dKO(#1)/WT]. The tumor growth curve of WT SCC7 injected into naive C3H/HeOu mice in FIG. 2D is also shown in light gray for reference (WT). Data are presented as means±SEM; n=8 tumors for each group. ***p<0.001, two-way ANOVA test. A schematic representation of the rechallenge experiment is shown in the lower panel. (E) WT or LATS1/2 dKO B16-OVA cells were transplanted into Rag-1 knockout (KO) mice that lack mature B and T lymphocytes. Tumor growth was monitored after the indicated times. Data are presented as means±SEM; n=8 tumors for each group. ns, not significant (p>0.05, two-way ANOVA test). (F) Kaplan-Meier tumor-free survival curves for mice transplanted as in (E) are shown (n=10 mice for each group). ns, not significant (p>0.05, log-rank test). (G) WT and LATS1/2 dKO B16-OVA melanoma cells were injected into opposite flanks in the same Rag-1 KO mouse. Data are presented as means±SEM; n=6 tumors for each group. The tumor growth curves shown in (E) are shown in a lighter color for reference. The p value was determined using a two-way ANOVA test, comparing the “WT [with LATS1/2 dKO(#1)]” group to the WT group. ns, not significant (p>0.05).

FIGS. 12A-12F. EVs Released from LATS1/2-Null Tumor Cells Stimulate Host Immune Responses (A) Bone marrow-derived dendritic cells (BMDCs) were pretreated with conditioned medium from WT or LATS1/2 dKO B16-OVA melanoma cells (or control medium) and pulsed with OVA protein. BMDCs were then subjected to an in vitro crosspresentation assay using CFSE-labeled CD8⁺ T cells isolated from OVA-specific T-cell-receptor transgenic OT-I mice. OT-ICD8⁺ T cells proliferate when stimulated with OVA antigen via crosspresentation by BMDCs, resulting in dilution of CFSE content. Representative histograms of the gated CD8⁺ T cells are shown in the left panel. The division index was calculated, and data are presented as means±SEM of three independent experiments in the right panel. ***p<0.001, one-way ANOVA test followed by Tukey's multiple comparison test. (B) BMDCs were stimulated with conditioned medium or EVs from WT or LATS1/2 dKO B16-OVA cell culture supernatants, and then IL-12 levels in the culture supernatants were determined by ELISA. Data are presented as means±SEM; n=3 independent experiments for conditioned medium stimulation, n=4 independent experiments for EVs stimulation. *p<0.05; ***p<0.001, one-way ANOVA test followed by Tukey's multiple comparison test. (C) C57BL/6 mice were inoculated with irradiated WT B16-OVA cells at the base of the tail, and EVs freshly isolated from culture supernatants of equal numbers of WT or LATS1/2 dKO B16-OVA cells were injected every 3 days (days 0, 3, 6, and 9). At day 12, mice were challenged with WT B16-OVA melanoma and tumor growth was monitored. Data are presented as means±SEM; n=8 tumors for each group. The tumor growth curves shown in FIG. 11B are presented in a lighter color for reference. The p value was determined using a two way ANOVA test, comparing WT EV-immunized group (WT+WT EVs/WT) to LATS1/2 dKO Evimmunized group (WT+LATS1/2 dKO EVs/WT). ***p<0.001. (D) EVs isolated from culture supernatants of equal numbers of WT or LATS1/2 dKO B16-OVA cells were subjected to nanoparticle tracking analysis (NanoSight) to quantify the number and size distribution. The numbers of particles are presented as means±SEM of three independent experiments. **p<0.01, unpaired t test. (E) Protein concentrations of EVs isolated as in (D) were determined. Data are presented as means±SEM of six independent experiments. ***p<0.001, unpaired t test. (F) EVs were isolated from culture supernatants of equal numbers of WT, LATS1/2 dKO, or YAP(5SA)-overexpressing B16-OVA cells, and RNA concentrations were determined by Agilent TapeStation. Data are presented as means±SEM of three independent experiments. ***p<0.001, one-way ANOVA test followed by Tukey's multiple comparison test. See also FIGS. 18-20.

FIG. 13A-13I. LATS1/2-Deleted Tumor EVs Stimulate Anti-tumor Immunity via the TLRs-Type I IFN Pathway (A-G) WT or LATS1/2 dKO B16-OVA cells were transplanted into mice deficient in Myd88 (A; n=12 mice for the WT group, and n=13 mice for the LATS1/2 dKO group), Ticam1 (also known as TRIF) (B; n=6 mice for each group), Tmem173 (also known as STING) (C; n=4 mice for each group), Casp1 (also known as caspase-1) (D; n=4 mice for the WT group, and n=5 mice for the LATS1/2 dKO group), Tlr4 (E; n=9 mice for each group), Tlr7 (F; n=8 mice for each group), or Tlr9 (G; n=7 mice for each group), and Kaplan-Meier tumor-free survival curves are shown. The survival curves of WT C57BL/6 mice injected with WT or LATS1/2 dKO B16-OVA in FIG. 9C are shown in a lighter color for reference. The p value was determined using a log-rank test, comparing KO mice injected with LATS1/2 dKO B16-OVA cells (orange) to corresponding wild-type C57BL/6 mice injected with LATS1/2 dKO B16-OVA cells (light red). ns, not significant (p>0.05); *p<0.05; **p<0.01; ***p<0.001. (H) WT or LATS1/2 dKO B16-OVA cells were injected into Ifnar1 KO mice that lack functional type I IFN receptor, and tumor growth was monitored after the indicated times. The tumor growth curves of WT or LATS1/2 dKO B16-OVA cells injected into wild-type C57BL/6 mice in FIG. 2A are shown in a lighter color for reference. Data are presented as means±SEM; n=8 tumors for each group. (I) Kaplan-Meier tumor-free survival curves for mice injected as in (H) are shown (n=8 mice for each group). The survival curves of WT C57BL/6 mice injected with WT or LATS1/2 dKO B16-OVA in FIG. 2C are shown in a lighter color for reference. The p value was determined using a log-rank test, comparing Ifnar1 KO mice injected with LATS1/2 dKO B16-OVA cells (orange) to corresponding WT C57BL/6 mice injected with LATS1/2 dKO B16-OVA cells (light red). ***p<0.001. See also FIG. 21.

FIG. 14. Proposed Model for the Regulation of Anti-tumor Immunity by the Hippo Pathway in Tumors Poorly immunogenic tumor cells evade host immune defenses, despite expressing antigenic neoepitopes. LATS1/2 deletion in tumor cells stimulates nucleicacid-rich EV secretion, which induces a type I IFN response via the TLRs-MYD88/TRIF pathway. Type I IFN stimulates multiple components of host immune responses, including cross-presentation of tumor-derived antigens by antigen-presenting cells and T cell activation. Activated T cells, in turn, facilitate tumor-specific responses of cytotoxic T cells and antibody production by B cells, promoting tumor destruction. Thus, loss of LATS1/2 in tumors leads to the rejection of both LATS1/2-deficient and LATS1/2-adequate tumor cells by enhancing host anti-tumor immune responses.

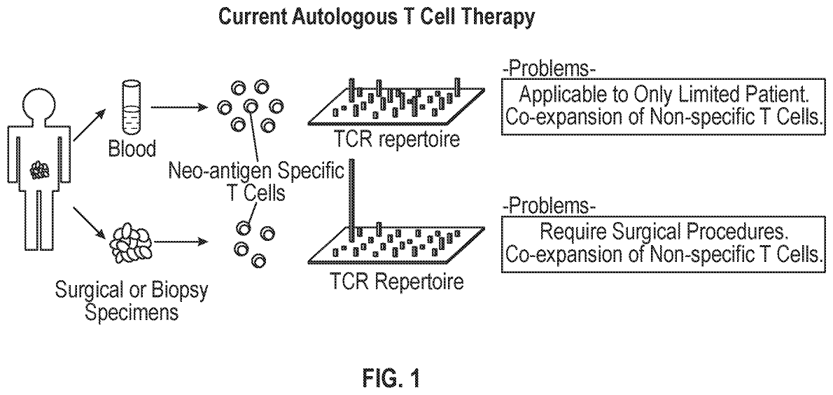

FIGS. 15A-15G. LATS1/2 Deletion Enhances Anchorage-Independent Tumor Cell Growth In Vitro, Related to FIG. 8. (A) LATS1/2 dKO B16-OVA cells grow similarly to WT on regular cell culture plates. Wild-type (WT) and two independent clones of LATS1/2 double knockout (dKO) B16-OVA melanoma cells (1×10⁵) were plated in 6-well culture dishes and cell number was determined with a hemocytometer after the indicated times. Data are means±SD of triplicate cultures from a representative experiment. ns, not significant (p>0.05, two-way ANOVA test). (B) Deletion of LATS1/2 in SCC7 cells abolishes YAP phosphorylation in response to actin polymerization inhibitor Latrunculin B (LatB) treatment and glycolysis inhibitor 2-deoxy-D-glucose (2-DG) treatment. WT and two independent clones of LATS1/2 dKO SCC7 squamous cell carcinoma cells were subjected to immunoblot (IB) analysis with antibodies to the indicated proteins. The mouse YAP S112 is equivalent to human YAP S127, which is the major regulatory site responsible for YAP cytoplasmic localization. Where indicated, gels containing phos-tag were employed for assessment of YAP phosphorylation status. YAP proteins can be separated into multiple bands in the presence of phos-tag depending on differential phosphorylation levels, with phosphorylated proteins migrating more slowly. (C) Loss of LATS1/2 promotes YAP/TAZ nuclear localization. LatB-treated or non-treated (control) SCC7 cells were subjected to immunostaining analysis. YAP/TAZ subcellular localization was determined by immunofluorescence staining for endogenous YAP/TAZ (green) along with D API for DNA (blue). Representative images are presented in the left panel. (Right panel) Cells in five randomly selected views (about 100 cells) were selected for the quantification of YAP/TAZ localization. N, nuclear; C, cytoplasmic. (D) LATS1/2 deletion promotes anchorage-independent growth of SCC7 cells in vitro. Representative images of the soft-agar colony-formation assay in FIG. 8D are shown. (E) Deletion of LATS1/2 in 4T1 cells abolishes YAP phosphorylation in response to serum starvation, LatB treatment, and 2-DG treatment. WT and LATS1/2 dKO 4T1 breast cancer cells were subjected to immunoblot and phos-tag analysis. (F) Loss of LATS1/2 promotes YAP/TAZ nuclear localization. LatB-treated or non-treated (control) 4T1 cells were subjected to immunostaining analysis. YAP/TAZ subcellular localization was determined by immunofluorescence staining for endogenous YAP/TAZ (green) along with DAPT for DNA (blue). Representative images are presented in the left panel. (Right panel) Cells in five randomly selected views (about 100 cells) were selected for the quantification of YAP/TAZ localization. N, nuclear; C, cytoplasmic. (G) LATS1/2 deletion promotes anchorage-independent growth of 4T1 cells in vitro. Representative images of the soft-agar colony-formation assay in FIG. 1E are shown.

FIGS. 16A-16C. Loss of LATS1/2 in Tumor Cells Inhibits Tumor Growth In Vivo, Related to FIG. 2 (A) Deletion of LATS1/2 in B16-OVA melanoma inhibits tumor growth in vivo. Wild-type (WT) or clone #2 of LATS1/2 double knockout (dKO) B16-OVA cells were injected into C57BL/6 mice and tumor weight was determined 16 days after transplantation. Data are represented as mean±SEM; n=6 tumors for each genotype. **p<0.01, Mann-Whitney test. (B) Deletion of LATS1/2 in SCC7 squamous cell carcinoma inhibits tumor growth in vivo. The gross appearance of C3H/HeOu mice injected with WT or two independent clones of LATS1/2 dKO SCC7 cells was examined 18 days after transplantation. (C) Deletion of LATS1/2 in 4T1 breast cancer inhibits tumor growth in vivo. The gross appearance of the primary tumors of WT or LATS1/2 dKO 4T1 cells injected into BALB/c mice was examined 28 days after transplantation.

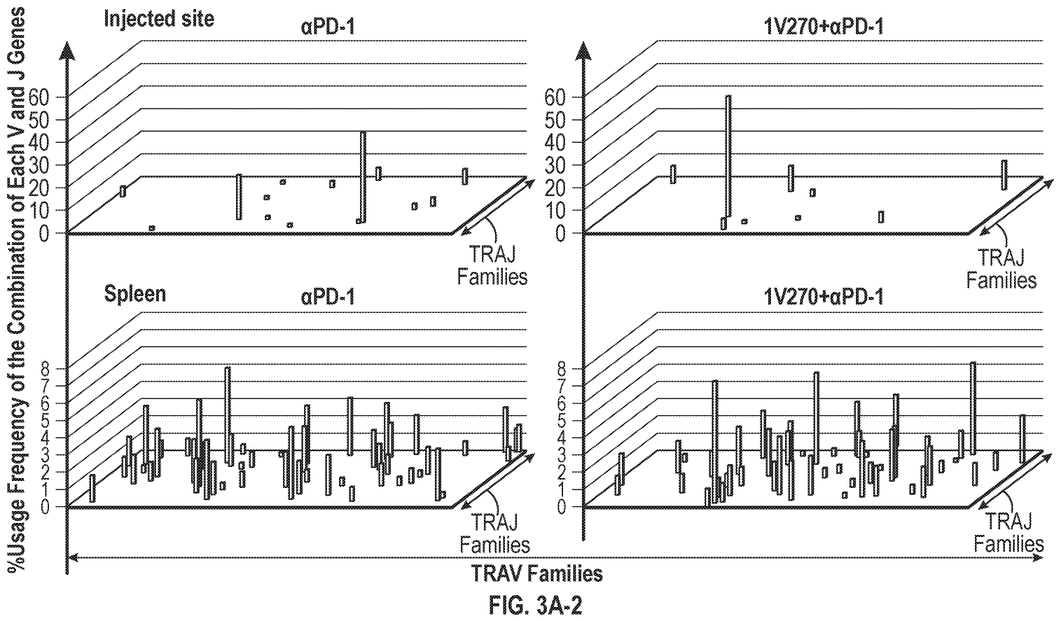

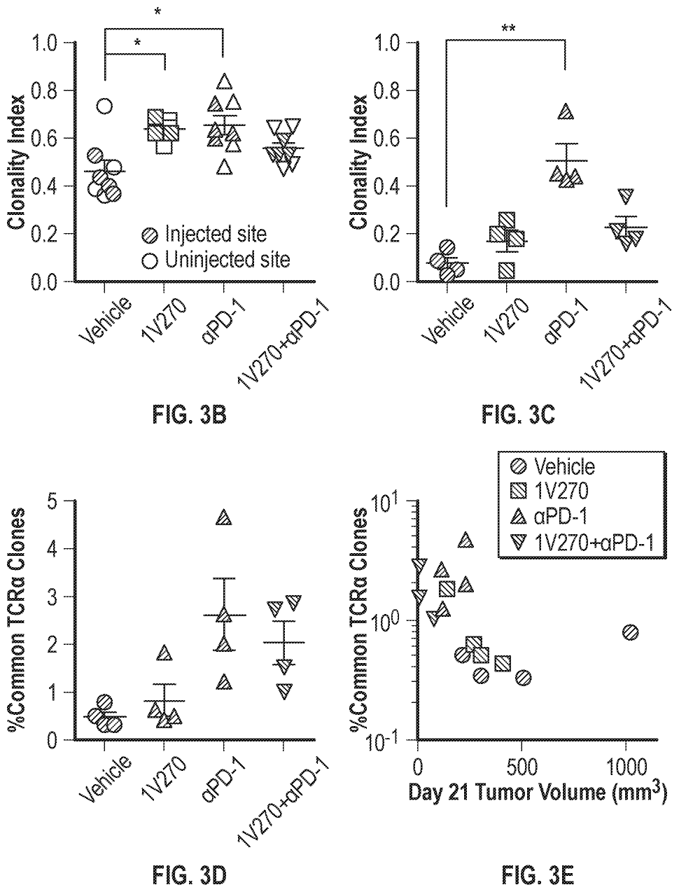

FIGS. 17A-17F. LATS1/2 Deficiency in Tumor Cells Stimulates Host Anti-tumor Immunity, Related to FIG. 10. (A) LATS1/2 deletion induces immune responses. Wild-type (WT) or LATS1/2 double knockout (dKO) 4T1 breast cancer cells were injected into BALB/c mice. Tumors were paraffin-embedded and stained with hematoxylin and eosin (H&E) 28 days after transplantation. Arrowheads indicate infiltration of inflammatory cells. (B) CD45+ leukocytes infiltrate into LATS1/2 null tumors. Frozen sections from WT or LATS1/2 dKO 4T1 breast cancers were subjected to immunostaining analysis of CD45 (red) along with DAPI for DNA (blue). (C) LATS1/2 null tumors activate CD8+ T cells. C57BL/6 mice were injected (or not) with WT or LATS1/2 dKO B16-OVA melanoma cells. 12 days after transplantation, splenocytes were collected and re-stimulated ex vivo with SIINFEKL peptide and then subjected to flow cytometric analysis. SIINFEKL is an OVA-derived peptide being presented through the class I major histocompatibility complex (MHC class I) molecule, H-2Kb. Representative scatterplots of the gated CD8+ T cells in FIG. 10E are shown. Gating of CD8+ T cells was performed after background assessment. Numbers indicate the percentage of Granzyme B or interferon γ (IFNγ) positive cells in the gated CD8+ population. FSC, forward scatter. (D) LATS1/2 deletion in tumors increases OVA-specific CD8+ T cells. Splenocytes from C57BL/6 mice injected as in (C) were subjected to flow cytometric analysis. OVA-specific CD8+ T cells were quantified using K^{b}-SIINFEKL-tetramers. Representative scatterplots of the gated CD8+ T cells in FIG. 10F are shown. Gating of CD8+ T cells was performed after background assessment. Numbers indicate the percentage of tetramer positive cells in the gated CD8+ population. (E) CD8+ T cells from LATS1/2 dKO tumor-challenged mice show increased OVA-specific cytotoxicity. C57BL/6 mice were injected (or not) with WT or LATS1/2 dKO B16-OVA melanoma cells and CD8+ T cells were isolated from splenocytes. T cell cytotoxicity assay was performed with CFSE-labeled EL4 cells ex vivo. The frequency of CSFE^{high} (irrelevant peptide control) and CFSE^{low} (SIINFEKL loaded target) EL4 cells was determined by flow cytometric analysis 18 hours after incubation. Representative histograms of the gated CSFE⁺ EL4 cells in FIG. 10H are shown in the upper panel. Gating of CSFE⁺ EL4 cells was performed after background assessment. Numbers indicate the percentage of the gated cells in CSFE⁺ population. Schematic representation of ex vivo cytotoxicity assay using CFSE-labeled EL4 cells is shown in the lower panel. CFSE, carboxy fluorescein succinimidyl ester. (F) CD8+ T cells infiltrate into LATS1/2 dKO tumors. WT or LATS1/2 dKO B16-OVA melanoma cells were injected into C57BL/6 mice and tumors were subjected to flow cytometric analysis 12 days after transplantation. Representative scatterplots of the gated CD45+ cells in FIG. 3I are shown. Gating of CD45+ T cells was performed after background assessment. Numbers indicate the percentage of CD8 positive cells in the gated CD45+ population.

FIGS. 18A-18H. Overexpression of YAP or TAZ in Tumor Cells Partially Suppresses Tumor Growth In Vivo, Related to FIG. 11. (A) YAP/TAZ activation in LATS1/2 null tumors. Wild-type (WT) or LATS1/2 double knockout (dKO) B16-OVA melanoma cells were injected into C57BL/6 mice. 20 days after transplantation, tumor samples were harvested and then subjected to immunoblot (IB) analysis with antibodies to the indicated proteins. YAP dephosphorylation and TAZ accumulation were evident in tumors deficient for LATS1/2. n=3 tumors for each group. (B) Increased YAP/TAZ transcriptional activity in LATS1/2 null tumors. The tumor samples, same as in (A), were subjected to RT and real-time PCR analysis of the indicated YAP/TAZ target gene mRNA. Normalized data are expressed relative to the corresponding value for WT tumors and are mean±SEM; n=3 tumors for each group. *p<0.05, unpaired t test. (C) Expression levels of YAP(5SA) or TAZ(4SA) in B16-OVA melanoma cells. YAP(5SA) and TAZ(4SA) are active mutants of YAP/TAZ with all five/four LATS1/2 phosphorylation sites mutated to alanine, thereby unresponsive to inhibition by the LATS1/2 kinase. B16-OVA cells stably expressing YAP(5SA), TAZ(4SA), or control vector were subjected to immunoblot analysis with antibodies to the indicated proteins. (D) YAP(5SA) or TAZ(4SA) overexpression promotes anchorage-independent growth of B16-OVA cells in vitro. Soft-agar colony-formation assay was performed and the colonies were stained with crystal violet for quantification. Data are means±SD from 3 independent experiments. **p<0.01; ***p<0.001, one-way ANOVA test followed by Tukey's multiple comparison test. (E) YAP(5SA) or TAZ(4SA) overexpression in B16-OVA melanoma inhibits tumor growth in vivo. B16-OVA cells stably expressing YAP(5SA), TAZ(4SA), or control vector were injected into C57BL/6 mice and tumor growth was monitored after the indicated times. Data are represented as mean±SEM; n=8 tumors for each group. p values were determined using two-way ANOVA test, comparing each group to control group. ***p<0.001. (F) Expression levels of YAP(5SA) or YAP(5SA/S94A) in B16-OVA melanoma cells. TEAD1-4 are the major YAP-associated transcription factors, and their binding to YAP requires the Ser94 residue in YAP. YAP(5SA/S94A) is unable to bind TEAD1-4 and thus fails to promote TEAD-dependent transcription. (G) YAP(5SA), but not YAP(5SA/S94A), promotes downstream target gene transcription in B16-OVA cells. Total RNA extracted from B16-OVA cells stably expressing the indicated constructs was subjected to RT and real-time PCR analysis of the indicated YAP/TAZ target genes. Data are means±SD of triplicates from a representative experiment. (H) Tumor growth suppression by YAP in vivo requires TEAD-binding of YAP. B16-OVA cells stably expressing YAP(5SA/S94A) were injected into C57BL/6 mice and tumor growth was monitored after the indicated times. The tumor growth curves shown in (E) are presented in a lighter color for reference. Data are represented as mean±SEM; n=8 tumors for each group. p value was determined using two-way ANOVA test, comparing YAP(5SA) group to YAP(5SA/S94A) group. ***p<0.001.

FIGS. 19A-19B. EVs Released from LATS1/2-Null Tumor Cells Stimulate Host Immune Responses, Related to FIG. 12. (A) Enrichment of the EV protein markers, such as CD81, ALIX, and FLOT1 in the EV preparation. Whole cell lysate (WCL) of wild-type (WT) or LATS1/2 double knockout (dKO) B16-OVA melanoma cells as well as EVs isolated from their culture supernatants were subjected to immunoblot (IB) analysis with antibodies to the indicated proteins. Equal amounts of protein samples (2.5 mg) were resolved in SD S-PAGE in non-reducing conditions. (B) Detergent treatment abolishes the activity of EV preparations in stimulating bone marrow-derived dendritic cells (BMDCs). Culture supernatants of WT or LATS1/2 dKO B16-OVA melanoma cells were either treated or untreated with detergent (1% Triton X-100) and then ultracentrifuged to isolate EVs. The resulting EV pellets were re-suspended in PBS and used to stimulate BMDCs. 18 hours after incubation, IL-12 levels in the culture supernatants of BMDC were determined by ELISA. Data are represented as mean±SEM of 3 independent experiments. p value was determined using one-way ANOVA test followed by Tukey's multiple comparison test. ***p<0.001; ns, not significant (p>0.05).

FIGS. 20A-20F. EVs Released from LATS1/2-Null Tumors Contain More Nucleic-Acid-Binding Proteins in Comparison to WT EVs, Related to FIG. 12. (A) LATS1/2 null tumor cells secrete more EVs. EVs isolated from culture supernatants of wild-type (WT) or LATS1/2 double knockout (dKO) B16-OVA melanoma cells were subjected to nanoparticle tracking analysis (NanoSight) to quantify the number and size distribution. Representative histograms in FIG. 12D are shown. (B) Proteomic profiling of total proteins identified in EVs show enrichment of previously reported exosomal and microvesicle cargo proteins. EVs were isolated from culture supernatants of WT or LATS1/2 dKO B16-OVA cells and subjected to mass spectrometry analysis. Enrichment analysis of the Gene Ontology (GO) cellular component of total EV proteins identified (1,772 proteins) was done using the PANTHER program. The enrichment p value of each term was transformed to a −log₁₀ (p value). The top 3 most significantly enriched cellular components are indicated. (C) Heatmap of the total EV proteins identified. Absolute protein abundances were estimated using the iBAQ algorithm. The iBAQ-scaled protein expression (Ex) was transformed to a log₂ (Ex) and the scale indicates relative expression. Data represent two independent biological replicates. See Table 51 for protein contents. (D) RNA binding and nucleic-acid-binding proteins are enriched in EVs from LATS1/2 null tumor cells. Enrichment analysis of the GO molecular function of the top 100 most significantly increased proteins in LATS1/2 dKO EVs is shown. The enrichment p value of each term was transformed to a−log₁₀ (p value). (E) EVs from LATS1/2-deficient tumor cells or YAP(5SA)-overexpressing tumor cells contain higher amounts of RNA than EVs from WT tumor cells. EVs were isolated from culture supernatants of equal numbers of WT, LATS1/2 dKO, or YAP(5SA)-overexpressing B16-OVA cells and RNA concentrations were determined by Agilent TapeStation. Representative histograms in FIG. 5F are shown. (F) EVs from B16-OVA cells contain single stranded RNA. EVs were isolated from culture supernatants of equal numbers of WT or LATS1/2 dKO B16-OVA cells. RNA was then purified from EV samples and either treated or untreated with single-strand-specific ribonuclease (RNase A, the reaction was performed under high salt concentrations to achieve single-strand specificity), followed by agarose gel electrophoresis in non-denaturing conditions.

FIGS. 21A-21D. EVs from LATS1/2-Deleted Tumor Cells Stimulate Anti-tumor Immunity via the TLRs-Type-I-IFN Pathway, Related to FIG. 13. (A) Schematic representation of the endogenous nucleic-acid-sensing pathway s and TLR signaling in mice (Junt and Barchet, 2015). TLR, toll-like receptor; LPS, lipopoly saccharide; dsRNA, double-stranded RNA; ssRNA, single-stranded RNA; rRNA, ribosomal RNA; TRIF, TIR-domain-containing adaptor-inducing interferon-b; MYD88, myeloid differentiation primary response 88; STING, stimulator of interferon genes; IL, interleukin; IFN, interferon. (B) Wild-type (WT) or LATS1/2 double knockout (dKO) B16-OVA cells were injected into Myd88 knockout (KO) mice and tumor growth was monitored after the indicated times. Data are represented as mean±SEM; n=8 tumors for each group. (C) WT or LATS1/2 dKO B16-OVA cells were injected into Ticam1 (also known as TRIF) KO mice and tumor growth was monitored after the indicated times. Data are represented as mean±SEM; n=10 tumors for each group. (D) LATS1/2 deletion has little effect on the mRNA abundance of type I IFN in tumor cells. WT or LATS1/2 dKO B16-OVA cells were stimulated (or not) with 2 mg/ml poly(I:C) complexed with 4 mg/ml Poly Jet for 24 hours. Total RNA was extracted from the cells and then subjected to RT and real-time PCR analysis of the indicated mRNA. Data are means±SD of triplicates from a representative experiment.

FIGS. 22A-22C. Systemic administration of 1V270 inhibits lung metastasis in the 4T1 murine syngeneic breast cancer model in a CD8⁺ T cell dependent manner. (A) Protocol of spontaneous metastasis model. 4T1 cells (5×10⁵) were inoculated in both 4th mammary pads of Balb/c mice (n=13/group) 1V270 (20, 80, 04 200 μg/injection) was administered on days 7, 10, 14, 17, 21 and 24. (B) The numbers of lung nodules were counted by staining with India ink after harvesting lungs on day 27. (C) The mice (n=6-15/group) were orthotopically implanted with 4T1 cells and i.p. treated with 1V270 (20 μg/injection) as shown in FIG. 1A. CD8⁺ cells were depleted by administration of anti-CD8 or isotype mAbs on days 5, 8, 11, 16, 19, and 23. The numbers of lung nodules were counted on day 27. Each dot indicates an individual mouse and horizontal and vertical bars indicate means±SEM. Data shown are pooled from 2 independent experiments. *P<0.05, **P<0.01 by Kruskal-Wallis test with Dunn's post hoc test comparing treatment groups against vehicle group. n.s; statistically not significant.

FIGS. 23A-23G. Systemic administration of 1V170 induces tumor-specific CD8⁺ T cells. (A) Protocol of IV metastasis model. (B) BALB/c mice (n=B-15/group) were i.v. injected with 4T1 cells (2×10⁴) on day 0.1V270 (2, 20, or 200 μg/injection) was i.p. administered on days −1, 7, 10, and 14. The numbers of lung nodules were counted on day 21. (C) 200 μg/injection 1V270 was i.p. administered on days −1 or 0 followed by days 7, 10, and 14 where indicated. The numbers of lung nodules were counted on day 21. Each dot indicates an individual mouse and horizontal and vertical bars indicate means±SEM. *P<0.05, **P<0.01 Kruskal-Wallis test with Dunn's post hoc test comparing treatment groups against vehicle group. (D-G) BALB/c mice (n=10/group) were treated with 1V270 (200 μg/injection) on day-1 and 4T1 cells were inoculated on day 0. (D,E) Three weeks later, mLNs cells were stained for CD3 and CD8. Intracellular granzyme B (D) and IFNγ (E) were analyzed by flow cytometry. Data are representative of 2 independent experiments showing similar results. *P<0.05, by the Mann-Whitney U test comparing the 1V270 treatment groups against the vehicle treated group. (F) Histological analysis of lungs on day 21. Representative images of H&E staining and immunohistochemical staining for CD3 and CD45. Scale bar: 100 μm. Original magnification ×200. (G) Tumor-specific cytotoxicity was examined using 4T1 cells as target cells and BALB/3T3 cells as irrelevant target cells. The splenocytes incubated with 4T1 cell lysate and IL-2 were cocultured with 4T1 and BALB/3T3 cells at 16:1 and 2:1 effector to target cell ratios (E:T), respectively, for 16 hours. The percent specific killing was calculated. Data were analyzed by two-way ANOVA using a Bonferroni post hoc test comparing treatment groups against vehicle group. *P<0.05. Data are representative of 3 independent experiments showing similar results.

FIGS. 24A-24G. Tumor infiltrating T cells in 1V270 treated mice show high clonalities and increased frequency of intra- and inter-individual common clones (A) Experimental protocols of the secondary challenge studies. Two groups of BALB/c mice (n=5/group) were i.p, treated with 1V270. One cohort of mice was i.v. injected with 4T1-GLF cells (2×10⁴) on day 0 and tumor growth in the lungs was monitored by IVIS on day 20. Another cohort did not receive i.v. tumor injection (no tumor-exposed mice). Native BALB/c mice served as controls. 4T1 cells were orthotopically inoculated on day 21. (B) Tumor growth was measured with a caliper and calculated using the formula: volume (mm3)=(width)2×length/2. (C) TILs s were isolated from the secondarily challenged tumor on day 39 and stained for CD8⁺ T cells (CD3⁺CD8⁺) and PD-1. The numbers of tumor-infiltrating cos• T cells were expressed per tumor volume (mm³). Data are representative of 2 independent experiments showing similar results. Results were analyzed by the Mann-Whitney U test comparing the 1V270 treatment groups against the vehicle treated group. **P<0.01. (D·G) TCR repertoire of CD8⁺ T cells from TILs s and splenocytes were examined. (D) The clonality index (1-normalized Shannon index) of CD8⁺ T cells infiltrating to the left side of the secondarily challenged tumor was plotted against tumor volume in the tumor exposed mice. Correlation between the clonality index and the tumor volume was evaluated by Pearson's method (R²=0.97, p<0.05). (El BUB overlap index of CD8⁺ T cells TCRα (green) or TCRβ (blue) between Tls and splenocytes in each individual. Higher BUB index shows higher similarity of TCR repertoire between TILs and splenocytes. Each point shows the BUB index of individual mouse. (F) Heat map of BUB overlap index of TCRα or TCRβ in the same group. The color scale bar on the left shows that white is zero and red is 1. (G) BUB overlap index of TCRα (green) or TCRβ (blue) between individual mice was plotted. Statistical analysis was performed by the Mann-Whitney U test for comparing two groups. *P<0.05. Each point represents the BUB overlap index of TCRα or TCRβ between pairs of individual mice in the same groups.

FIGS. 25A-25F. Innate immune cells, NK cells and dendritic cells are activated following 1V270 therapy and contribute to anti-metastatic effects (A-C) Systemic administration of 1V270 activates dendritic cells and increases CD8⁺ cell recruitment in the lung draini lymph node. (A) BALB/c mice (n=S/group) were treated with 1V270 on day −1 and then tumor cells were i.v. administered on day 0. Seven days later, mediastinal lymph node (mLN) cells were stained for dendritic cells (DC; CD45⁺CD11c⁺MHC classll⁺). (B) DCs were further stained for CD80 and CD86. (C) The mLN cells were also stained for central memory CD8⁺ T cells (CD3+CD8⁺CD44⁺CD62L⁺), effector memory CD8⁺ T cells (CD3+CD8⁺CD44⁺CD62L⁺), and naive CD8⁺ T cells (CD3+CD8⁺CD44⁻CD62L⁻). Each dot indicates an individual mouse and horizontal and vertical bars indicate means±SEM. Data are representative of 3 independent experiments showing similar results. *P<0.05, **P<0.01 by Mann-Whitney U test comparing the individual groups. (D-F) In vivo imaging analyses using GLF•expressing 4T1 cells (4T1-GLF). (D) BALB/c mice (n=14-15/group) were i.p, administered with 200 μg 1V270 or vehicle. Next day, 2×10⁴ 4T1-GLF cells were i.v. injected through the tail vein. Tumor signals were quantified by IVIS. Data (mean±SEM) were pooled from 3 independent experiments showing similar results. *P<0.05, **P<0.01 by two-way ANOVA using a Bonferroni post hoc test comparing treatment groups against the vehicle group. The data are displayed as radiance on a color bar with a range of 1×10⁵ to 1×10⁶. (E,F) NK cells recruited by 1V270 therapy prevent colonization of 4T1 cells. (E) BALB/c mice (n=G−7/group) were treated with 1V270 (200 μg/injection) on day −1 and then tumor cells were i.v. administered on day 0. On the next day, lung cells were stained for NK markers (CD45⁺CD3⁺ NKp46⁺CD49⁺) and analyzed by flow cytometry. Mann-Whitney U test was used to comp are treatment groups against the vehicle group. **P<0.01. (F) 1V270 treated BALB/c mice (n=10/group) received 4T1 cells i.v. on day 0. NK cells were depleted by administration of anti-asialo-GM1 antibody (S0 μg/injection) on day −4, and −1. Tumor signals were analyzed as described above. Data (mean±SEM) were pooled from 2 independent experiments showing similar results. *P<0.05 by two-way ANOVA using Bonferroni post hoc test was used to comp are treatment groups against the vehicle in the first 24 hours. n.s.: not significant

FIGS. 26A-26D. Intranasally administered 1V270 inhibits pulmonary colonization in experimental metastasis models (A) Protocol for testing the therapeutic efficacy of i.n. treatment with 1V270 in IV metastasis model. BALB/c mice (n=6-8/group) were i.n. administered with 1V270 (20 or 200 μg) or vehicle on days −3, −1, 3, 7, and 10. 4T1 cells were v. injected on day 0. (B) The numbers of lung nodules were counted on Day 21. *P<0.05 calculated by Kruskal-Wallis test with Dunn's post hoc test. (C) Intranasal 1V270 therapy attenuated the growth of secondarily challenged tumors. BALB/c mice (n=S) were i.n. treated with 200 μg 1V270 on days −3, −1, 3, 7 and 10. 4T1 cells were injected on day 0. On day 21, the surviving mice were orthotopically inoculated with 4T1 cells and tumor growth was monitored. The mice treated with 1V270 without i.v. tumor cell injection served as controls. (D) TILs were isolated from the secondarily challenged tumor, and the numbers of CD8⁺ T cells and PD-1 expressing CD8⁺ T cells were analyzed by flow cytometric assay. Data were analyzed by the Mann-Whitney U test comparing two groups. *P<0.05. Data are representative of 2 independent experiments showing similar results.

FIGS. 27A-27D. Systemic 1V270 therapy effectively inhibits lung colonization in melanoma and Lewis lung cell carcinoma (LLC) models. Therapeutic effects of 1V270 were evaluated in IV metastatic models of 816 melanoma (A, B) and LLC (C, D). BG-albino mice (n=B-10) (A, C), C57BL6 wild type mice (n=15-20) (B and D), were i.p. administered with 200 μg 1V270 or vehicle!iNext day, 5×10⁵ B16-GLF cells (A) or 1×10⁵ LLC-GLF cells (c) were i.v. administered (A,C). The tumor signals were quantified by IVIS at day 14. The data are displayed as a radiance on a color bar with a range of 1×10⁶ to 1×10⁷ to 1×10⁶ (C). (B,D) The mice were monitored daily and were euthanized upon reaching the criteria according to UCSD IACUC guidelines. The survival data were analyzed by Log-Rank test. Data were pooled from 2 independent experiments showing similar results and were analyzed by Living Image® software. Each dot indicates an individual mouse and horizontal bars represent means. Statistical differences were analyzed by the Mann-Whitney U test comparing 1V270 treatment groups against the vehicle. **P<0.05.

FIGS. 28A-28C. Systemic administration of 1V270 inhibits lung metastasis, but not the primary tumor growth (A) Growth curves of the orthotopically implanted primary tumor in the spontaneous metastasis model. 4T1 cells (5×10⁵) were inoculated to both 4th mammary pads of BALB/c mice (n=I 3/group). 1V270 (20, 80, or 200 μg/injection) was i.p. administered on days 7, 10, 14, 17, 21, and 24 as shown in FIG. 1A. Tumor growth was measured with a caliper and calculated using the formula: volume (mm³)=(width)²×length/2. (B) Experimental protocol of T cell depletion. BALB/c mice (n=6-15/group) were i.p. treated with anti-CDS-mAb. (C) Growth curves of the orthotopically implanted primary tumor in the CD⁸⁺ T cells depleted mice. Data shown are mean±SEM and representative of two independent experiments showing similar data.

FIGS. 29A-29B. Representative gating process of CD8⁺ T cells in TILs by flow cytometry (A) Representative gating process of CD8⁺ T cells (CD45+CD3⁺CD8⁺). (B) Representative flow cytometric plots of CD8⁺ T cells on day 26 following CD8⁺ T cell depletion. Over 80% CD8⁺ T cell population was depleted both in vehicle and 1V270 treated groups.

FIGS. 30A-30C. 1V270 therapy increases frequency of commonly shared clones between individuals (A) The clonality index (I-normalized Shannon index) of CD8⁺ T cells infiltrating to left side of the secondarily challenged tumor were plotted against both sides of tumor volume in the no tumor exposed mice. The correlation was evaluated by a Pearson's correlation test (R²=0.15, p=0.61). (B) The Venn diagram of “shared clones” among the individual mice in the same group. Based on the sequence of CDR3 region, number of TCR clones shared between individual mice were counted. The shared clone was defined as the TCR clone consisting of identical V and D genes and amino acid sequence of CDR3 shared among 3 or more mice in the groups. (C) The percentage frequency of shared clones in the total reads in TILs. The frequency was calculated by dividing the sum of number of sequence reads from shared clones by the total number of reads. Each dot represents the frequency of the shared clones of either TCR α (green) or TCRβ (blue) in the individual mice.

FIGS. 31A-31C. Systemic administration of 1V270 activates local dendritic cells in the lungs (A) Representative gating process of CD11c+MHC classll+ dendritic cells in the lungs. (B) BALB/c mice (n=S/group) were treated with 1V270 (200 μg/injection) on day −1 and then tumor cells were i.v. administered on day 0. Seven days later, single cell suspensions derived from the lungs were stained for CD1 k′MHC class Ir′ dendritic cells (left panel). The cells were further assayed for costimulatory molecules (CD80, and CD86) expression (right panel). Each dot indicates an individual mouse and horizontal and vertical bars indicate means±SEM. *P<0.05, **P<0.0 I by Kruskal-Wallis test with Dunn's post hoc test comparing treatment groups against vehicle.

FIGS. 32A-32B. In vivo imaging in the IV metastasis model (A) In vivo imaging protocol in IV metastasis model. (B) Representative of lung tumor signals by IVIS in FIG. 4D.

FIGS. 33A-33C. 1V270 therapy recruits innate immune cells to the lungs. (A-C) Representative flow cytometric plots and gating process of immune cells in the lungs. BALB/c mice (n=6-7/group) were treated with 1V270 (200 μg/injection) on day −1 and then tumor cells were i.v. administered on day 0. Next day, single cell suspensions derived from the lungs were stained for (A) NK cells (CD45⁺CD3⁻NKp46⁺CD49⁺), (B) M-MDSCs (CD45⁺CD11b⁺Ly6G⁻Ly6^{high}) and (C) PMN-MDSCs (CD45⁺CD11b⁺Ly6G⁺) were analyzed by flow cytometric assay. (C) Frequencies of M-MDSCs and PMN-DSCs were compared between 1V270 treated and vehicle-treated mice. Each dot indicates an individual mouse and horizontal and vertical bars indicate means±SEM. Data are representative of 2 independent experiments showing similar results. Mann-Whitney U test was used to comp are treatment groups against the vehicle group. *P<0.05, **P<0.01.

FIGS. 34A-34C. Antibody-mediated depletion of NK cells reverses inhibition of tumor cell colonization by 1V270 therapy in the early phase. (A) Experimental protocol of NK cell depletion. Anti-asialo GMI rabbit polyclonal Ab (aGM1) or rabbit IgG poly clonal Ab was injected on days −4, −1, 3, and 10. 1V270 (200 μg/injection) was administered on day −1 and 2×10⁴ 4T1 cells were i.v. injected on day 0. (B) Representative flow cytometric plots of NK cell frequency in the lung on day 14. (C) Representative of lung tumor signals of NK cell depleted mice by IVIS in FIG. 4F.

FIG. 35. Toll-like receptor 7 expression in 4T1, B16, and LLC cells. Expression of TLR7 in the cell lines used in this study was assessed by quantitative RT-PCR.

FIG. 36. Systemic administration of 1V270 induces significantly lower levels of cytokine induction by systemically administrated 1V270 and 1V136. BALB/c mice (n=5) were i.p. administered IV270 (200 μg=185 nmol/injection) or 2V136 (58 μg=185 nmol/injection) and sera were collected 2, 4, 6, and 24 hours following the administration. The levels of TNFα, IL-12, and IP-IO were measured by Luminex assay. *P<0.05, **P<0.01 by two way ANOVA using a Bonferroni post hoc test comparing treatment groups.

FIGS. 37A-37B. The correlation between the tumor signal in the lung at day 10, and the number of lung nodules or overall survival. (A) BALB/c mice (n=S) were injected with 2×10⁴ 4T1 cells on day 0. Tumor cell signals were monitored using IVIS. Tumor signals on day 10 were plotted with the number of lung tumor nodules examined on day 21. (B) BALB/c mice (n=8/group) were injected with 1V270 on day −1 and with 2×10⁴ 4T1 cells on day 0. Tumor signals were monitored using IVIS. Tumor signals on day 10 were plotted with survival (days) of individual mice. The representative tumor signal images by IVIS for (A) and (B) are shown at right. The correlation was evaluated by a Pearson's correlation test.

### DETAILED DESCRIPTION

### Definitions

A “phospholipid” as the term is used herein refers to a glycerol mono- or diester bearing a phosphate group bonded to a glycerol hydroxyl group with an alkanolamine group being bonded as an ester to the phosphate group, of the general formula

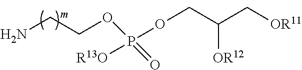

wherein R¹¹ and R¹² are each independently hydrogen or an acyl group, and R¹³ is a negative charge or a hydrogen, depending up on pH. When R13 is a negative charge, a suitable counterion, such as a sodium ion, can be present. For example, the alkanolamine moiety can be an ethanolamine moiety, such that m=1. It is also understood that the NH group can be protonated and positively charged, or unprotonated and neutral, depending up on pH. For example, the phospholipid can exist as a zwitterion with a negatively charged phosphate oxy anion and a positively charged protonated nitrogen atom. The carbon atom bearing OR¹² is a chiral carbon atom, so the molecule can exist as an R isomer, an S isomer, or any mixture thereof. When there are equal amounts of R and S isomers in a sample of the compound of formula (II), the sample is referred to as a “racemate.” For example, in the commercially available product 1,2-dioleoyl-sn-glycero-3-phosphoethanolamine, as used in Example I below, the R³ group is of the chiral structure

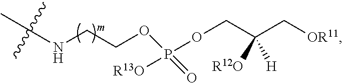

which is of the R absolute configuration.

A phospholipid can be either a free molecule, or covalently linked to another group for example as shown

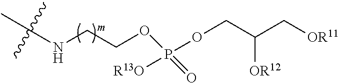

wherein a wavy line indicates a point of bonding.

Accordingly, when a substituent group, such as R³ of the compound of formula (I) herein, is stated to be a phospholipid what is meant that a phospholipid group is bonded as specified by the structure to another group, such as to an N-benzyl heterocyclic ring system as disclosed herein. The point of attachment of the phospholipid group can be at any chemically feasible position unless specified otherwise, such as by a structural depiction. For example, in the phospholipid structure shown above, the point of attachment to another chemical moiety can be via the ethanolamine nitrogen atom, for example as an amide group by bonding to a carbonyl group of the other chemical moiety, for example

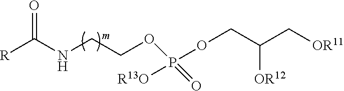

wherein R represents the other chemical moiety to which the phospholipid is bonded. In this bonded, amide derivative, the R¹³ group can be a proton or can be a negative charge associated with a counterion, such as a sodium ion. The acylated nitrogen atom of the alkanolamine group is no longer a basic amine, but a neutral amide, and as such is not protonated at physiological pH.

An “acyl” group as the term is used herein refers to an organic structure bearing a carbonyl group through which the structure is bonded, e.g., to glycerol hydroxyl groups of a phospholipid, forming a “carboxylic ester” group. Examples of acyl groups include fatty acid groups such as oleoyl groups, that thus form fatty (e.g, oleoyl) esters with the glycerol hydroxyl groups. Accordingly, when R¹¹ or R¹², but not both, are acyl groups, the phospholipid shown above is a mono-carboxylic ester, and when both R¹¹ and R¹² are acyl groups, the phospholipid shown above is a di-carboxylic ester.

Within the present disclosure it is to be understood that a compound of the formula (I) or a salt thereof may exhibit the phenomenon of tautomerism whereby two chemical compounds that are cap able of facile interconversion by exchanging a hydrogen atom between two atoms, to either of which it forms a covalent bond. Since the tautomeric compounds exist in mobile equilibrium with each other they may be regarded as different isomeric forms of the same compound. It is to be understood that the formulae drawings within this specification can represent only one of the possible tautomeric forms. However, it is also to be understood that the disclosure encompasses any tautomeric form, and is not to be limited merely to any one tautomeric form utilized within the formulae drawings. The formulae drawings within this specification can represent only one of the possible tautomeric forms and it is to be understood that the specification encompasses all possible tautomeric forms of the compounds drawn not just those forms which it has been convenient to show graphically herein. For example, tautomerism may be exhibited by a pyrazolyl group bonded as indicated by the wavy line. While both substituents would be termed a 4-pyrazolyl group, it is evident that a different nitrogen atom bears the hydrogen atom in each structure.

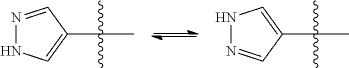 [SMILES: *c1cn[nH]c1 ; *c1cn[nH]c1]

Such tautomerism can also occur with substituted pyrazoles such as 3-methyl, 5-methyl, or 3,5-dimethylpyrazoles, and the like. Another example of tautomerism is amido-imido (lactam-lactim when cyclic) tautomerism, such as is seen in heterocyclic compounds bearing a ring oxygen atom adjacent to a ring nitrogen atom. For example, the equilibrium:

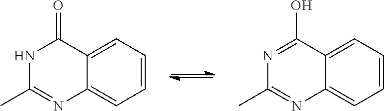

is an example of tautomerism.
Accordingly, a structure depicted herein as one tautomer is intended to also include the other tautomer.

### Optical Isomerism

It will be understood that when compounds of the present disclosure contain one or more chiral centers, the compounds may exist in, and may be isolated as pure enantiomeric or diastereomeric forms or as racemic mixtures. The present disclosure therefore includes any possible enantiomers, diastereomers, racemates or mixtures thereof of the compounds of the disclosure.

The isomers resulting from the presence of a chiral center comprise a pair of non-superimposable isomers that are called “enantiomers.” Single enantiomers of a pure compound are optically active, i.e., they are capable of rotating the plane of plane polarized light. Single enantiomers are designated according to the Cahn-Ingold-Prelog system. The priority of substituents is ranked based on atomic weights, a higher atomic weight, as determined by the systematic procedure, having a higher priority ranking Once the priority ranking of the four groups is determined, the molecule is oriented so that the lowest ranking group is pointed away from the viewer. Then, if the descending rank order of the other groups proceeds clockwise, the molecule is designated (R) and if the descending rank of the other groups proceeds counterclockwise, the molecule is designated (S). In the example in Scheme 14, the Cahn-Ingold-Prelog ranking is A>B>C>D. The lowest ranking atom, D is oriented away from the viewer.

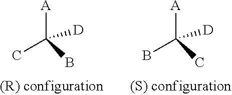

The present disclosure is meant to encompass diastereomers as well as their racemic and resolved, diastereomerically and enantiomerically pure forms and salts thereof. Diastereomeric p airs may be resolved by known separation techniques including normal and reverse phase chromatography, and crystallization.

“Isolated optical isomer” means a compound which has been substantially purified from the corresponding optical isomer(s) of the same formula. In one embodiment, the isolated isomer is at least about 80%, e.g., at least 90%, 98% or 99% pure, by weight.

Isolated optical isomers may be purified from racemic mixtures by well-known chiral separation techniques. According to one such method, a racemic mixture of a compound of the disclosure, or a chiral intermediate thereof, is separated into 99% wt. % pure optical isomers by HPLC using a suitable chiral column, such as a member of the series of DAICEL® CHIRALPAK family of columns (Daicel Chemical Industries, Ltd., Tokyo, Japan). The column is operated according to the manufacturer's instructions.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, behenic, salicylic, sulfanilic, 2-acetoxybenzoic, fumaric, toluenesulfonic, methanesulfonic, ethane disulfonic, oxalic, isethionic, and the like.

The pharmaceutically acceptable salts of the compounds useful in the present disclosure can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile may be employed. Lists of suitable salts are found in Remington's Pharmaceutical Sciences, 17th ed., Mack Publishing Company, Easton, Pa., p. 1418 (1985), the disclosure of which hereby incorporated by reference.

The compounds of the formulas described herein can be solvates, and in some embodiments, hydrates. The term “solvate” refers to a solid compound that has one or more solvent molecules associated with its solid structure. Solvates can form when a compound is crystallized from a solvent. A solvate forms when one or more solvent molecules become an integral part of the solid crystalline matrix up on solidification. The compounds of the formulas described herein can be solvates, for example, ethanol solvates. Another type of a solvate is a hydrate. A “hydrate” likewise refers to a solid compound that has one or more water molecules intimately associated with its solid or crystalline structure at the molecular level. Hydrates can form when a compound is solidified or crystallized in water, where one or more water molecules become an integral part of the solid crystalline matrix.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

The following definitions are used, unless otherwise described: halo or halogen is fluoro, chloro, bromo, or iodo. Alkyl, alkoxy, alkenyl, alkynyl, etc. denote both straight and branched groups; but reference to an individual radical such as “propyl” embraces only the straight chain radical, a branched chain isomer such as “isopropyl” being specifically referred to. Aryl denotes a phenyl radical or an ortho-fused bicyclic carbocyclic radical having about nine to ten ring atoms in which at least one ring is aromatic. Het can be heteroaryl, which encompasses a radical attached via a ring carbon of a monocyclic aromatic ring containing five or six ring atoms consisting of carbon and one to four heteroatoms each selected from the group consisting of non-peroxide oxygen, sulfur, and N(X) wherein X is absent or is H, O, (C₁-C₄)alkyl, phenyl or benzyl, as well as a radical of an ortho-fused bicyclic heterocycle of about eight to ten ring atoms derived therefrom, particularly a benz-derivative or one derived by fusing a propylene, trimethylene, or tetramethylene diradical thereto.

It will be appreciated by those skilled in the art that compounds of the disclosure having a chiral center may exist in and be isolated in optically active and racemic forms. Some compounds may exhibit polymorphism. It is to be understood that the present disclosure encompasses any racemic, optically-active, polymorphic, or stereoisomeric form, or mixtures thereof, of a compound of the disclosure, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine agonist activity using the standard tests described herein, or using other similar tests which are well known in the art. It is also understood by those of skill in the art that the compounds described herein include their various tautomers, which can exist in various states of equilibrium with each other.

### Methods to Enhance Cancer Specific CD8+ Cells

Cellular transformation, tumor growth, and metastasis constitute a multistep process that requires the continuous rewiring of signaling pathway s and alterations of the reciprocal interaction between cancer cells and the tumor microenvironment, thereby allowing cells to acquire features to become fully neoplastic and eventually malignant (Hanahan and Weinberg, 2011). The Hippo pathway has gained great interest in recent y ears as being strongly involved in several of these key hallmarks of cancer progression (Harvey et al., 2013; Moroishi et al., 2015a) and, in general, serves important regulatory functions in organ development, regeneration, and stem cell biology (Johnson and Halder, 2014; Yu et al., 2015). The heart of the mammalian Hippo pathway is a kinase cascade involving mammalian STE20-like protein kinase 1 (MST1; also known as STK4) and MST2 (also known as STK3) (homologs of Drosophila Hippo), as well as two groups of MAP4Ks (mitogen-activated protein kinase kinase kinase kinases)-MAP4K1/2/3/5 (homologs of Drosophila Happy hour) and MAP4K4/6/7 (homologs of Drosophila Misshapen)—and the large tumor suppressor 1 (LATS1) and LATS2 (homologs of Drosophila Warts) (Meng et al., 2016). When the Hippo pathway is activated, MST1/2 or MAP4Ks phosphorylate and activate the LATS1/2 kinases, which, in turn, directly phosphorylate and inactivate Yes-associated protein (YAP) and transcriptional coactivator with PDZ-binding motif (TAZ; also known as WWTR1), the two major downstream effectors that mediate transcriptional output of the Hippo pathway (Hansen et al., 2015). Activation of LATS1/2 kinases (and inactivation of YAP/TAZ) rep resents the major functional output of the Hippo pathway.

Previous studies have convincingly established the Hippo pathway as a suppressor signal for cellular transformation and tumorigenesis, though other studies revealed its oncogenic functions in certain contexts (Moroishi et al., 2015a; Wang et al., 2014). Deletion of MST1/2 in mouse liver results in tissue overgrowth and tumor development, demonstrating the tumor suppressor function of these kinases (Zhou et al., 2009). Complementarily, overexpression of YAP in mouse liver also promotes tissue overgrowth and tumorigenesis (Camargo et al., 2007; Dong et al., 2007). These studies have demonstrated an inhibitory role of the Hippo pathway in tumor initiation. However, effects of the Hippo pathway in tumor growth, especially in the context of reciprocal interactions between tumor cells and host anti-tumor immune responses, remain largely unknown.

Recent advances in cancer immunotherapy have improved patient survival. However, only a minority patients with pulmonary metastatic disease responds to treatment with checkpoint inhibitors. As an alternate approach, we have tested the ability of systemically administered 1V270, a toll-like receptor 7 (TLR7) agonist conjugated to a phospholipid, to inhibit lung metastases in two variant murine 4T1 breast cancer models, as well as in B16 melanoma, and Lewis lung c models. In the 4T1 breast cancer models, 1V270 therapy inhibited lung metastases if given up to a week after primary tumor initiation. The treatment protocol was facilitated by the minimal toxic effects exerted by the phospholipid TLR7 agonist, compared to the unconjugated agonist. The 1V270 therapy inhibited colonization by tumor cells in the lungs in a NK cell dependent manner. Additional experiments revealed that single administration of 1V270 led to tumor-specific CD8⁺ cell-dependent adaptive immune responses that suppressed late stage metastatic tumor growth in the lungs. T cell receptor (TCR) repertoire analyses showed that 1V270 therapy induced oligoclonal tumor-specific T cells in the lungs and regional lymph nodes. Different animals displayed commonly shared TCR clones following 1V270 therapy. Intranasal administration of 1V270 also suppressed lung metastasis and induced tumor-specific adaptive immune responses. These results indicate that systemic 1V270 therapy can induce tumor-specific cytotoxic T cell responses to pulmonary metastatic cancers, and that TCR repertoire analyses can be used to monitor, and to predict, the response to therapy.

Exemplary TLR7 ligands are shown below.

In one embodiment, the TLR7 ligand has formula (I):

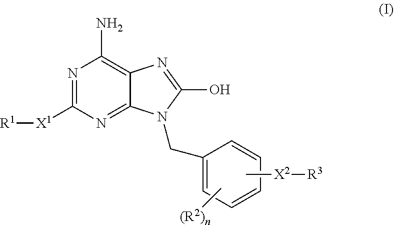

wherein X¹ is —O—, —S—, or —NR^{c}—;

R¹ is hydrogen, (C₁-C₁₀)alkyl, substituted (C₁-C₁₀)alkyl, C₆₋₁₀aryl, or substituted C₆₋₁₀aryl, C₅₋₉heterocyclic, substituted C₅₋₉heterocyclic;

R^{c} is hydrogen, C₁₋₁₀alkyl, or substituted C₁₋₁₀alkyl; or R^{c} and R¹ taken together with the nitrogen to which they are attached form a heterocyclic ring or a substituted heterocyclic ring;

each R² is independently —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl (aroyl), substituted —C(O)—(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), halo, nitro, or cyano, or R² is absent;

each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl;

wherein the substituents on any alkyl, aryl or heterocycle groups are hydroxy, C₁₋₆alkyl, hydroxy C₁₋₆alkylene, C₁₋₆alkoxy, C₃₋₆cycloalkyl, C₁₋₆alkoxyC₁₋₆alkylene, amino, cyano, halo, or aryl;

n is 0, 1, 2, 3 or 4;

X² is a bond or a linking group; and

R³ is a phospholipid comprising one or two carboxylic esters;

or a tautomer thereof;

or a pharmaceutically acceptable salt or solvate thereof.

In one embodiment, the composition of the disclosure comprises nanoparticles comprising a compound of formula (I). As used herein, a nanoparticle has a diameter of about 30 nm to about 600 nm, or a range with any integer between 30 and 600, e.g, about 40 nm to about 250 nm, including about 40 to about 80 or about 100 nm to about 150 nm in diameter. The nanoparticles may be formed by mixing a compound of formula (I), which may spontaneously form nanoparticles, or by mixing a compound of formula (I) with a preparation of lipids, such as phospholipids including but not limited to phosphatidylcholine, phosphatidylserine or cholesterol, thereby forming a nanoliposome. Optionally, a compound of formula (I), a lipid preparation and a glycol such as propylene glycol are combined.

In one embodiment, a composition comprises an amount of a compound of Formula (I):

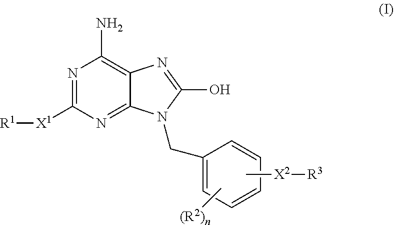

wherein X¹ is —O—, —S—, or —NR^{c}—;

R¹ is hydrogen, (C₁-C₁₀)alkyl, substituted (C₁-C₁₀)alkyl, C₆₋₁₀aryl, or substituted C₆₋₁₀ aryl, C₅₋₉ heterocyclic, substituted C₅₋₉ heterocydic;

R^{c} is hydrogen, C₁₋₁₀alkyl, or substituted C₁₋₁₀alkyl; or R^{c} and R¹ taken together with the nitrogen to which they are attached form a heterocycle ring or a substituted heterocyclic ring;

each R² is independently —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl(aroyl), substituted —C(O)—(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), halo, nitro, or cyano, or R² is absent;

each R^{a} and R^{b} is independently hydrogen, (C₁-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl; wherein the substituents on any alkyl, aryl or heterocyclic groups are hydroxy, C₁₋₆alkyl, hydroxy C₁₋₆alkylene, C₁₋₆alkoxy, C₃₋₆cycloalkyl, C₁₋₆alkoxyC₁₋₆alkylene, amino, cyano, halo, or aryl;
n is 0, 1, 2, 3 or 4;

X² is a bond or a linking group; and

R³ is a phospholipid comprising one or two carboxylic esters;

or a tautomer thereof;

or a pharmaceutically acceptable salt or solvate thereof. Optionally, the composition further comprises an antigen. In one embodiment, the composition having an antigen is administered concurrently, prior to or subsequent to administration of the composition having a compound of formula (I).

For example, R³ can comprise a group of formula

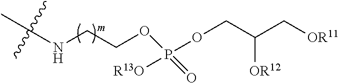

wherein R¹¹ and R¹² are each independently a hydrogen or an acyl group, R¹³ is a negative charge or a hydrogen, and m is 1 to 8, wherein a wavy line indicates a position of bonding, wherein an absolute configuration at the carbon atom bearing OR¹² is R, S, or any mixture thereof.

For example, m can be 1, providing a glycerophosphatidylethanolamine. More specifically, R¹¹ and R¹² can each be oleoyl groups.

In various embodiments, the phospholipid of R³ can comprise two carboxylic esters and each carboxylic ester includes one, two, three or four sites of unsaturation, epoxidation, hydroxylation, or a combination thereof.

In various embodiments, the phospholipid of R³ can comprise two carboxylic esters and the carboxylic esters of are the same or different. More specifically, each carboxylic ester of the phospholipid can be a C17 carboxylic ester with a site of unsaturation at C8-C9. Alternatively, each carboxylic ester of the phospholipid can be a C18 carboxylic ester with a site of unsaturation at C9-C10.

In various embodiments, X² can be a bond or a chain having one to about 10 atoms in a chain wherein the atoms of the chain are selected from the group consisting of carbon, nitrogen, sulfur, and oxygen, wherein any carbon atom can be substituted with oxo, and wherein any sulfur atom can be substituted with one or two oxo groups. The chain can be interspersed with one or more cycloalkyl, aryl, heterocyclyl, or heteroaryl rings.

In various embodiments, X² can be C(O), or can be any of

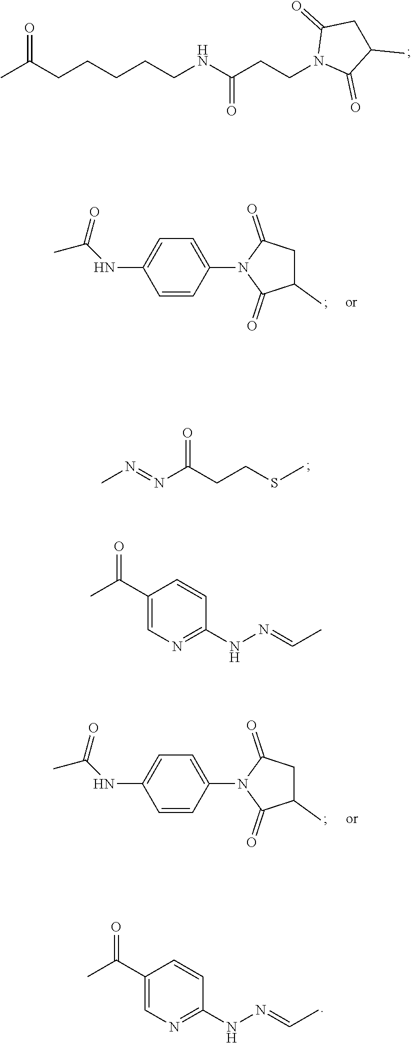

In various embodiments, R³ can be dioleoylphosphatidyl ethanolamine (DOPE).

In various embodiments, R³ can be 1,2-dioleoyl-sn-glycero-3-phospho ethanolamine and X² can be C(O).

In various embodiments, X¹ can be oxygen.

In various embodiments, X¹ can be sulfur, or can be —NR^{c}— where R^{c} is hydrogen, C₁₋₆ alkyl or substituted C₁₋₆ alkyl, where the alkyl substituents are hydroxy, C₃₋₆cycloalkyl, C₁₋₆alkoxy, amino, cyano, or aryl. More specifically, X¹ can be —NH—.

In various embodiments, R¹ and R^{c} taken together can form a heterocyclic ring or a substituted heterocyclic ring. More specifically, R¹ and R^{c} taken together can form a substituted or unsubstituted morpholino, piperidino, pyrrolidino, or piperazino ring.

In various embodiments R¹ can be a C1-C10 alkyl substituted with C1-6 alkoxy.

In various embodiments, R¹ can be hydrogen, C₁₋₄alkyl, or substituted C₁₋₄alkyl. More specifically, R¹ can be hydrogen, methyl, ethyl, propyl, butyl, hydroxy C₁₋₄alkylene, or C₁₋₄alkoxy C₁₋₄alkylene. Even more specifically, R¹ can be hydrogen, methyl, ethyl, methoxyethyl, or ethoxy ethyl.

In various embodiments, R² can be absent, or R² can be halogen or C₁₋₄alkyl. More specifically, R² can be chloro, bromo, methyl, or ethyl.

In various embodiments, X¹ can be O, R¹ can be C₁₋₄alkoxy-ethyl, n can be 1, R² can be hydrogen, X² can be carbonyl, and R³ can be 1,2-dioleoylphosphatidyl ethanolamine (DOPE).

In various embodiments, the compound of Formula (I) can be:

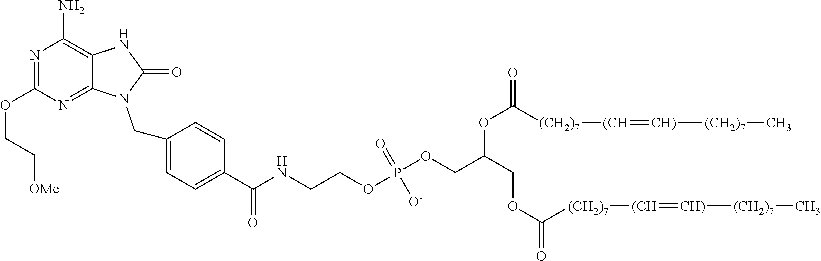

In various embodiments, the compound of formula (I) can be the R— enantiomer of the above structure:

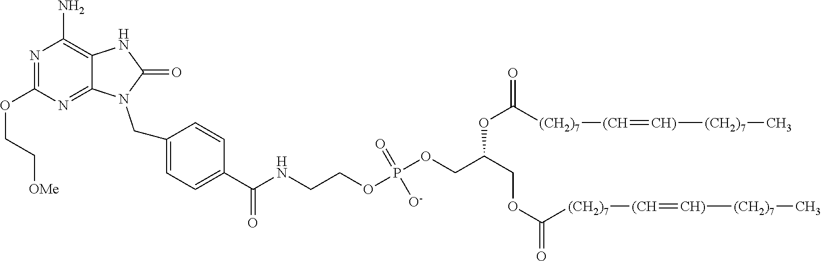

In one embodiment, the TLR& ligand is:

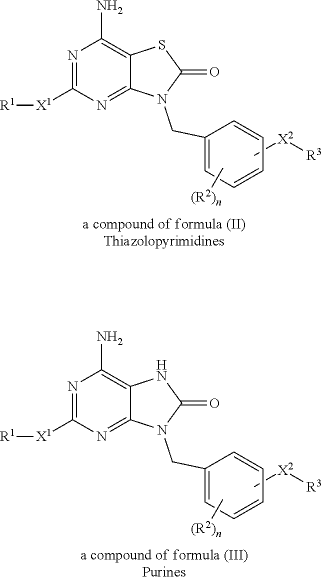

X¹═—O—, —S—, or —NR^{c}—,

wherein R^{c} hydrogen, C₁₋₁₀alkyl, or C₁₋₁₀alkyl substituted by C₃₋₆ cycloalkyl, or R^{c} and R¹ taken together with the nitrogen atom can form a heterocyclic ring or a substituted heterocyclic ring wherein the substituents are hydroxy, C₁₋₆ alkyl, hydroxy C₁₋₆ alkylene, C₁₋₆ alkoxy, C₁₋₆ alkoxy C₁₋₆ alkylene, or cyano;

wherein R¹ is (C₁-C₁₀)alkyl, substituted (C₁-C₁₀)alkyl, C₆₋₁₀ aryl, or substituted C₆₋₁₀ aryl, C₅₋₉ heterocyclic, substituted C₅₋₉ heterocyclic; wherein the substituents on the alkyl, aryl or heterocyclic groups are hydroxy, C₁₋₆ alkyl, hydroxy C₁₋₆ alkylene, C₁₋₆ alkoxy, C₁₋₆ alkoxy C₁₋₆ alkylene, amino, cyano, halogen, or aryl;

each R² is independently —OH, (C₁-C₆)alkyl, substituted (C₁-C₆)alkyl, (C₁-C₆)alkoxy, substituted (C₁-C₆)alkoxy, —C(O)—(C₁-C₆)alkyl (alkanoyl), substituted —C(O)—(C₁-C₆)alkyl, —C(O)—(C₆-C₁₀)aryl(aroyl), substituted —C(O)—(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), —O—C(O)NR^{a}R^{b}, —(C₁-C₆)alkylene-NR^{a}R^{b}, —(C₁-C₆)alkylene-C(O)NR^{a}R^{b}, halo, nitro, or cyano;

wherein each R^{a} and R^{b} is independently hydrogen, (C₁₋₆)alkyl, (C₃-C₈)cycloalky, (C₁₋₆6)alkoxy, halo(C₁₋₆)alkyl, (C₃-C₈)cycloalkyl(C1-6)alkyl, (C₁₋₆)alkanoyl, hydroxy(C₁₋₆)alkyl, aryl, aryl(C₁₋₆)alkyl, aryl, aryl(C₁₋₆)alkyl, Het, Het (C₁₋₆)alkyl, or (C₁₋₆)alkoxycarbonyl; wherein X² is a bond or a linking group; wherein R³ is a phospholipid comprising one or two carboxylic esters wherein n is 0, 1, 2, 3, or 4; or a tautomer thereof; or a pharmaceutically acceptable salt thereof.

In cases where compounds are sufficiently basic or acidic to form acid or base salts, use of the compounds as salts may be appropriate. Examples of acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Alkyl includes straight or branched C₁₋₁₀ alkyl groups, e.g., methyl, ethyl, propyl, butyl, pentyl, isopropyl, isobutyl, 1-methylpropyl, 3-methylbutyl, hexyl, and the like.

Lower alkyl includes straight or branched C₁₋₆ alkyl groups, e.g., methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 1,1-dimethylpropyl, 1,2-dimethylpropyl, 2,2-dimethylpropyl, and the like.

The term “alkylene” refers to a divalent straight or branched hydrocarbon chain (e.g., ethylene: —CH₂—CH₂—).

C₃₋₇ cycloalkyl includes groups such as, cyclopropyl, cyclopentyl, cyclohexyl, cycloheptyl, and the like, and alkyl-substituted C₃₋₇ cycloalkyl group, preferably straight or branched C₁₋₆ alkyl group such as methyl, ethyl, propyl, butyl or pentyl, and C₅₋₇ cycloalkyl group such as, cyclopentyl or cyclohexyl, and the like.

Lower alkoxy includes C₁₋₆ alkoxy groups, such as methoxy, ethoxy or prop oxy, and the like.

Lower alkanoyl includes C₁₋₆ alkanoyl groups, such as formyl, acetyl, propanoyl, butanoyl, pentanoyl or hexanoyl, and the like.

C₇₋₁₁ aroyl, includes groups such as benzoyl or naphthoyl;

Lower alkoxy carbonyl includes C₂₋₇ alkoxy carbonyl groups, such as methoxy carbonyl, ethoxy carbonyl or prop oxy carbonyl, and the like.

Lower alkylamino group means amino group substituted by C₁₋₆ alkyl group, such as, methylamino, ethylamino, propylamino, butylamino, and the like.

Di(lower alkyl)amino group means amino group substituted by the same or different and C₁₋₆ alkyl group (e.g., dimethylamino, diethylamino, ethylmethylamino).

Lower alkylcarbamoyl group means carbamoyl group substituted by C₁₋₆ alkyl group (e.g., methylcarbamoyl, ethylcarbamoyl, propylcarbamoyl, butylcarbamoyl).

Di(lower alkyl)carbamoyl group means carbamoyl group substituted by the same or different and C₁₋₆ alkyl group (e.g., dimethylcarbamoyl, diethylcarbamoyl, ethylmethylcarbamoyl).

Halogen atom means halogen atom such as fluorine atom, chlorine atom, bromine atom or iodine atom.

Aryl refers to a C₆₋₁₀ monocyclic or fused cyclic aryl group, such as phenyl, indenyl, or naphthyl, and the like.

Heterocyclic or heterocycle refers to monocyclic saturated heterocyclic groups, or unsaturated monocyclic or fused heterocyclic group containing at least one heteroatom, e.g., 0-3 nitrogen atoms NR^{c}, 0-1 oxygen atom (—O—), and 0-1 sulfur atom (—S—). Non-limiting examples of saturated monocyclic heterocyclic group includes 5 or 6 membered saturated heterocyclic group, such as tetrahydrofuranyl, pyrrolidinyl, morpholinyl, piperidyl, piperazinyl or pyrazolidinyl. Non-limiting examples of unsaturated monocyclic heterocyclic group includes 5 or 6 membered unsaturated heterocyclic group, such as furyl, pyrrolyl, pyrazolyl, imidazolyl, thiazolyl, thienyl, pyridyl or pyrimidinyl. Non-limiting examples of unsaturated fused heterocyclic groups includes unsaturated bicyclic heterocyclic group, such as indolyl, isoindolyl, quinolyl, benzothiazolyl, chromanyl, benzofuranyl, and the like. A Het group can be a saturated heterocyclic group or an unsaturated heterocyclic group, such as a heteroaryl group.

R^{c} and R¹ taken together with the nitrogen atom to which they are attached can form a heterocyclic ring. Non-limiting examples of heterocyclic rings include 5 or 6 membered saturated heterocyclic rings, such as 1-pyrrolidinyl, 4-morpholinyl, 1-piperidyl, 1-piperazinyl or 1-pyrazolidinyl, 5 or 6 membered unsaturated heterocyclic rings such as 1-imidazolyl, and the like.

The alkyl, aryl, heterocyclic groups of R¹ can be optionally substituted with one or more substituents, wherein the substituents are the same or different, and include lower alkyl; cycloalkyl, hydroxyl; hydroxy C₁₋₆ alkylene, such as hydroxymethyl, 2-hydroxyethyl or 3-hydroxypropyl; lower alkoxy; C₁₋₆ alkoxy C₁₋₆ alkyl, such as 2-methoxyethyl, 2-ethoxyethyl or 3-methoxypropyl; amino; alkylamino; dialkyl amino; cyano; nitro; acyl; carboxyl; lower alkoxycarbonyl; halogen; mercapto; C₁₋₆ alkylthio, such as, methylthio, ethylthio, propylthio or butylthio; substituted C₁₋₆ alkylthio, such as methoxyethylthio, methylthioethylthio, hydroxyethylthio or chloroethylthio; aryl; substituted C₆₋₁₀ monocyclic or fused-cyclic aryl, such as 4-hydroxyphenyl, 4-methoxyphenyl, 4-fluorophenyl, 4-chlorophenyl or 3,4-dichlorophenyl; 5-6 membered unsaturated heterocyclic, such as furyl, pyrrolyl, pyrazolyl, imidazolyl, thiazolyl, thienyl, pyridyl or pyrimidinyl; and bicyclic unsaturated heterocyclic, such as indolyl, isoindolyl, quinolyl, benzothiazolyl, chromanyl, benzofuranyl or phthalimino. In certain embodiments, one or more of the above groups can be expressly excluded as a substituent of various other groups of the formulas.

The alkyl, aryl, heterocyclic groups of R² can be optionally substituted with one or more substituents, wherein the substituents are the same or different, and include hydroxyl; C₁₋₆ alkoxy, such as methoxy, ethoxy or propoxy; carboxyl; C₂₋₇ alkoxycarbonyl, such as methoxycarbonyl, ethoxy carbonyl or propoxycarbonyl) and halogen.

The alkyl, aryl, heterocyclic groups of R^{c} can be optionally substituted with one or more substituents, wherein the substituents are the same or different, and include C₃₋₆ cycloalkyl; hydroxyl; C₁₋₆ alkoxy; amino; cyano; aryl; substituted aryl, such as 4-hydroxyphenyl, 4-methoxyphenyl, 4-chlorophenyl or 3,4-dichlorophenyl; nitro and halogen.

The heterocyclic ring formed together with R^{c} and R¹ and the nitrogen atom to which they are attached can be optionally substituted with one or more substituents, wherein the substituents are the same or different, and include C₁₋₆ alkyl; hydroxy C₁₋₆ alkylene; C₁₋₆ alkoxy C₁₋₆ alkylene; hydroxyl; C₁₋₆ alkoxy; and cyano. A specific value for X¹ is a sulfur atom, an oxygen atom or —NR^{c}—.

In other embodiments, the TLR7 ligand has formula (I) wherein R³ is hydrogen, (C₁-C₁₀)alkyl, substituted (C₁-C₁₀)alkyl, C₆₋₁₀aryl, or substituted C₆₋₁₀aryl, C₅₋₉heterocyclic, substituted C₅₋₉heterocyclic.

In other embodiments, the TLR7 ligand has formula (I), wherein R³ is independently —OH, (C₁-C₆)alkyl, substituted (C₁-C₆)alkyl, (C₁-C₆)alkoxy, substituted (C₁-C₆)alkoxy, —C(O)—(C₁-C₆)alkyl(alkanoyl), substituted —C(O)—(C₁-C₆)alkyl, —C(O)—(C₆-C₁₀)aryl (aroyl), substituted —C(O)—(C₆-C₁₀)aryl, —C(O)OH (carboxyl), —C(O)O(C₁-C₆)alkyl (alkoxycarbonyl), substituted —C(O)O(C₁-C₆)alkyl, —NR^{a}R^{b}, —C(O)NR^{a}R^{b} (carbamoyl), halo, nitro, or cyano, or R² is absent; each R^{a} and R^{b} is independently hydrogen, (C1-C₆)alkyl, substituted (C₁-C₆)alkyl, (C₃-C₈)cycloalkyl, substituted (C₃-C₈)cycloalkyl, (C₁-C₆)alkoxy, substituted (C₁-C₆)alkoxy, (C₁-C₆)alkanoyl, substituted (C₁-C₆)alkanoyl, aryl, aryl(C₁-C₆)alkyl, Het, Het (C₁-C₆)alkyl, or (C₁-C₆)alkoxycarbonyl; wherein the substituents on any alkyl, aryl or heterocyclic groups are hydroxy, C₁₋₆alkyl, hydroxy C1-6 alkylene, C₁₋₆alkoxy, C₃₋₆cycloalkyl, C₁₋₆alkoxy C1-6alkylene, amino, cyano, halo, or aryl.

The invention will be further described by the following non-limiting examples.

### Example 1

The present disclosure provides several discoveries. First, the local administration of an immune stimulating agent (specifically a TLR7 or TLR9 agonist) to a syngeneic cancer in a mouse can cause the clonal expansion of tumor specific CD8⁺ T cells in TILs and spleen, as detected by RNA sequencing of T cell receptor variable region genes (FIG. 2). Second, the population of common T cell clones correlates with the clinical efficacy of the immune stimulating TLR therapy, and with the efficacy of checkpoint inhibitor therapy using an anti-PD-1 monoclonal antibody (FIG. 3). These results strongly suggest that clonal expansion of the tumor specific T cell is a biomarker for an immune reaction against the cancer (common clones between tumors and spleens in FIG. 3). The data also leads to the proposal that the tumor specific T cell receptor variable region gene products induced by a drug or other therapy can identify clonal TIL cells in the blood, permitting their more efficient isolation and expansion. Thus, drug treatment may cause the release of tumor specific T cell clones in TILs from the tumor, and their subsequent expansion in the blood and lymphoid tissues, outside of the suppressive tumor microenvironment.

In a separate series of experiments, it was found that inactivation in cancer cells of two enzymes, called LATS1 and LATS2, markedly increases the response of the immune system to the cancers, leading to tumor eradication in several examples (FIGS. 4 and 5). Without causing any detectable cytotoxic effects, the reduction in enzyme activity induces the release from the malignant cells of small extracellular vesicles (EVs) (e.g., less than about 0.2 microns in diameter). After isolation by filtration and ultracentrifugation, the small EVs initiate and activate tumor specific T cells in vivo (FIG. 6). When re-administered to mice, the isolated tumor EVs trigger a therapeutic immune response against the cancer, without detectable cytotoxicity to normal cells. Previous investigations have shown that various drugs, radiation, and heat shock can induce the release of EVs from tumor cells (Andaloussi et al., 2013; Vader et al., 2014). However, in almost every instance, the EV release was associated with cytotoxicity, exactly opposite to what is described herein. Moreover, most previous studies have shown that EVs released from cancers are immune suppressive and actually promote metastasis, rather than causing cancer specific immune stimulation, as described herein (Moroishi et al., 2016). Based upon these unexpected revelations, specific drugs that promote the release of immunogenic EVs from cancer cells, in the absence of cytotoxicity, are predicted to induce clonal expansion of tumor specific CD8+ T cells, and can be identified by the same protocols used in experiments with TLR agonists, without knowing the exact antigen specific for the tumor cells. The drugs need not be locally administered to a cancer. In one embodiment, the agents could be administered orally, parenterally, or by inhalation. The latter route may be particularly useful in patients with lung cancer or pulmonary metastases.

After drug therapy, the isolation and expansion of the tumor specific CD8⁺ T cells in tissue culture can be made much more efficient by purification of activated CD8⁺ T cells, and by co-culture of the T cells with the immunogenic tiny EVs derived from the blood of the same patient in the presence or absence of feeder cells. Moreover, the treatment of cancer patients with anti-PD1 and other checkpoint inhibitors should be undertaken specifically in those subjects who have circulating clonal T cells after drug treatment, and at the time when both EVs and TIL concentrations in the blood reach maximal levels.

To identify drugs that can induce immunogenic EV release with discernible toxic effects, in one embodiment, a mouse melanoma cell line is contacted with the drugs, a maximal non-toxic concentration is identified, released EVs with less than 0.2 micron diameter into the medium are measured, and the ability of the released EVs to stimulate tumor reactive T cells and to cause clonal expansion is assessed.

In contrast to current methods for the isolation of tumor specific autologous CD8⁺ T cells, which typically requires the processing of surgical specimens or biopsies to release the Tumor Infiltrating Lymphocytes (TILs) (FIG. 1), the method described herein does not require tissue processing. In addition, although previously in a few patients, tumor specific T cells have been identified in the blood, by their expression of PD-1, of activation antigens, and by reactivity with synthetic peptides derived from mutated oncogene products e.g., Ras (Arafeh et al., 2015), the presently disclosed method does not require any specific knowledge of the tumor antigens

Further, the current method for the expansion of TILs in tissue culture before re-infusion often leads to the co-expansion of non-specific T cells that can outgrow the effector T cells against the cancer. This problem can lead to therapeutic failure. In contrast, the presently disclosed methods involve a combination of drug treatments that enhances tumor specific T cells in the peripheral circulation before isolation and expansion of T cells in tissue culture, combined with stimulation with autologous immune EVs that contain tumor antigens and immunostimulatory molecules. This app roach renders TIL therapy much more efficient and less expensive.

In one embodiment, the following protocol may be employed:
1. Administration of a non-toxic drug that induces EVs release and expansion of tumor specific T cells in TILs to a patient, e.g., via the oral, parenteral, or intrapulmonary routes. The drugs may include TLR agonists, enzyme inhibitors, antibiotics, hormones, and the like.
2. Before, and each day after drug administration, up to about 14 days, a heparinized blood sample (10 mL) is withdrawn. PD-1 positive CD8⁺ T cells are isolated, for example, using magnetic beads, and the TCR alpha and beta mRNAs are reverse transcribed, and subjected to Nexgen RNA sequencing. The clonal expansion of tumor specific T cells is demonstrated by the increasing clonal dominance of one or more TCR alpha and TCR beta sequence mRNAs, compared to the pre-treatment specimen.
3. Small EVs in the pre-treatment and post-treatment blood specimens are isolated by filtration of plasma through an about 0.2 up to about 0.4 micron sterile filter, followed by high speed centrifugation. RNA and protein content are assessed.
4. At the time of substantial EV release, and TCR clonal dominance, anti-PD-1 antibody treatment is initiated.
5. The activated clonal CD8⁺ T cells are isolated, e.g., using beads coated with anti-TCR antibodies or by limiting dilution and tissue culture. T cells are expanded as previously described (Dudley et al., 2003). Briefly, T cells are expanded with a rapid expansion protocol using, for instance, OKT3 (anti-CD3) antibody and IL2, e.g., about 30 ng/mL OKT3 (anti-CD3) antibody and about 6,000 IU/mL IL2, in the presence of irradiated allogeneic feeder cells. The tissue cultures are supplemented once at initiation with EVs from the treated patients.
6. T cell cultures are expanded using cytokines, to yield 100 million to one billion cells; their TCR clonality may be confirmed.
7. In patients who did not responds fully to checkpoint inhibitor therapy as described above, the expanded TILs can be reinfused.

The disclosure will significantly improve isolation and expansion of autologous tumor specific T cells for cancer immunotherapy. Biopsies or surgical resection will no longer be required. Patients who are likely to respond to the T cell therapy will be identified early, before expansion of autologous T cells. Then all patients with metastatic cancer will be potential candidates for treatment.

### Example 2

In the present study, the role of the LATS1/2 kinases in the growth of established tumors in the context of anti-tumor immunity was investigated. Surprisingly, inactivation of the “tumor suppressor” LATS1/2 in tumor cells strongly suppresses tumor growth in immune-competent, but not immune-compromised, mice due to the induction of host anti-tumor immune responses. The data indicate a new paradigm for how tumor immunogenicity is regulated through the Hippo signaling pathway in tumor cells and also have implications for targeting LATS1/2 in cancer immunotherapy.

### Experimental Model and Subject Details

### Animals

C57BL/6, C3H/HeOu, or BALB/c mice were purchased from The Jackson Laboratory (Bar Harbor, Me., USA). Myd88, Tlr4, Tlr7, and Tlr9KO mice were kind gifts from Dr. Shizuo Akira (Osaka University, Osaka, Japan). Ticam1 (also known as TRIF) KO mice were kindly provided by Dr. Bruce Beutler (University of Texas Southwestern Medical Center, Dallas, Tex., USA). Casp1 (also known as Caspase-1) KO mice were kindly provided by Dr. Richard A. Flavell (Yale University School of Medicine, New Haven, Conn., USA). Rag1 KO mice, (also known as STING) KO mice, and OT-Itransgenic mice were purchased from The Jackson Laboratory. Ifnar1 KO mice were purchased from B&K Universal (East Yorkshire, United Kingdom). These mouse strains were backcrossed for 10 generations onto the C57BL/6 background at the University of California, San Diego. Mutant mice were bled by the University of California, San Diego Animal Care Program. 7-12 weeks old mice were used and all animal experiments were approved by the University of California, San Diego, Institutional Animal Care and Use Committee.

### Method Details

### Cell Culture and Gene Deletion by CRISPR/Cas9 System

All cell lines were cultured under an atmosphere of 5% CO₂ at 37° C. B16-OVA cells (B16F10 cells expressing ovalbumin) were cultured in DMEM (GIBCO) supplemented with 10% fetal bovine serum (FBS, GIBCO), penicillin (100 U/ml), and streptomycin (100 mg/ml). SCC7, 4T1, EL4, bone marrow-derived dendritic cells (BMDCs), mouse primary lymph node cells and CD8⁺ T cells were cultured in RPMI 1640 (GIBCO) supplemented with 10% FBS (GIBCO), penicillin (100 U/ml), and streptomycin (100 mg/ml).

LATS1/2-deficient cells were created through the CRISPR (clustered regularly interspaced short palindromic repeats)/Cas9 system (Ran et al., 2013). We use a transient CRISPR strategy for the deletion of LATS1/2 to avoid any potential unspecific effects mediated by stable Cas9/sgRNA genome integration. Cells were transiently transfected with a Cas9 and single-guide RNA (sgRNA) expression plasmid encoding puromycin resistance (PX459; Addgene plasmid #48139). The CRISPR-transfected cells will thus acquire transient resistance to puromycin. The guide sequences were designed using the Optimized CRISPR Design at http://crispr.mit.edu. The guide sequences used are 5′-AGACGTTCTGCTCCGAAATC-3′ (SEQ ID NO:1) or 5′-ACGTTTCCATTGGCGAATGA-3′ (SEQ ID NO:2) for mouse Lats1; 5′-GAGTGTCCAGCTTACAAGCG-3′ (SEQ ID NO:3) or 5′-GCTGGGTGGTGCAAACTACG 3′ (SEQ ID NO:4) for mouse Lats2.

Following transfection and transient selection with puromycin for 3 days, cells were single-cell sorted by fluorescence-activated cell sorting (FACS) into 96-well plate without puromycin. Knockout clones were selected by immunoblot analysis for the lack of LATS1/2 proteins and YAP phosphorylation. LATS1/2 dKO cells were sensitive to puromycin after expansion, indicating a transient expression of CRISPR/Cas9 system in those cells. Two independent clones were analyzed as indicated and the parental LATS1/2 WT cells (not transfected with PX459) were used as control.

### Retroviral Infection

B16-OVA cells stably expressing empty vector, YAP(5SA), YAP(5SA/S94A), or TAZ(4SA) were generated by retroviral infection. 293 Phoenix retrovirus packaging cells were transfected with pBABE empty vector, pBABE YAP(5SA), pBABE YAP (5SA/S94A), or pBABE TAZ (4 SA) constructs. Forty-eight hours after transfection, retroviral supernatant was supplemented with 5 mg/mL polybrene, filtered through a 0.45 mm filter, and used for infection. Forty-eight hours after infection, cells were selected with 4 mg/mL puromycin in culture medium.

### Immunoblot Analysis

Equal amount of protein samples were resolved in SDS-PAGE in reducing conditions unless otherwise mentioned in the Figure Legends. Antibodies to YAP (#14074), pYAP (S127 in humans and S112 in mice; #4911), YAP/TAZ (#8418), LATS1 (#3477), CD81 (#10037), and ALIX (#2171) were obtained from Cell Signaling those to actin (# ab3280) and ovalbumin (OVA, # ab1221) were from Abcam; those to LATS2 (# A300-479A, also weakly recognize LATS1) were from Bethyl Laboratories; those to FLOT1 (#610821) and HSP90 (#610418) were from BD Biosciences. The phos-tag electrophoresis was performed as described previously (Moroishi et al., 2015b). YAP proteins can be separated into multiple bands in the presence of phos-tag depending on differential phosphorylation levels, with phosphorylated proteins migrating more slowly. Where indicated, cells were treated with serum starvation (DMEM or RPMI 1640 without other supplements), 1 mg/ml Latrunculin B (LatB), or 25 mM 2-deoxy-D-glucose (2-DG) for 1 hour before harvest.

### Immunostaining of Cells

Cells were treated with or without 1 mg/ml Latrunculin B (LatB) for 1 hour before harvest. Cells were then fixed for 10 minutes at room temperature with 4% paraformaldehyde in phosphate-buffered saline (PBS) and were permeabilized with 0.1% Triton X-100 in PBS for 10 minutes at room temperature. Cells were then incubated consecutively with primary antibodies to YAP/TAZ (Santa Cruz, # sc-101199) (overnight at 4° C.) and Alexa Fluor 488-labeled goat secondary antibodies (for 90 minutes at room temperature) in PBS containing 1% bovine serum albumin (BSA). Cells were covered with a drop of ProLong Gold antifade reagent with DAPI (Invitrogen) for observation. Cells in five randomly selected views (about 100 cells) were selected for the quantification of YAP/TAZ localization.

### Reverse Transcription (RT) and Real-Time PCR Analysis

Total RNA (500 ng) isolated from cells with the use of RNeasy Plus Mini Kit (QIAGEN) was reverse-transcribed to complementary DNA using iScript cDNA Synthesis Kit (Bio-Rad). Complementary DNA was then diluted and used for quantification by real-time PCR, which was performed using KAPA SYBR FAST qPCR Kit (Kapa Biosystems) and the 7300 real-time PCR system (Applied Biosystems). The sequences of the PCR primers (forward and reverse, respectively) are 5′-GCCTGGAGAAACCTGCCAAGTATG-3′ (SEQ ID NO:5) and 5′-GAGTGGGAGTTGCTGTTGAAGTCG-3′ (SEQ ID NO:6) for mouse Gapdh; 5′-AGCTGACCTGGAGGAAAACA-3′ (SEQ ID NO:7) and 5′-GACAGGCTTGGCGATTTTAG-3′ (SEQ ID NO:8) for mouse Ctgf; 5′-GCTCAGTCAGAAGGCAGACC-3′ (SEQ ID NO:9) and 5′-GTTCTTGGGGACACAGAGGA-3′ (SEQ ID NO:10) for mouse Cyr61; 5′-AGGAGAAGAGTTGCCCACCTATGAG-3′ (SEQ ID NO:11) and 5′-TCGAAGAGCTTCATCCTGTCGC-3′ (SEQ ID NO:12) for mouse Amotl2; 5′-CCTGAGAAAGAAGAAACACAGCCTC-3′ (SEQ ID NO:13) and 5′-GCAAGTTGGTTGAGGAAGAGAGGG-3′ (SEQ ID NO:14) for mouse Ifna4; 5′-GAAGAGTTACACTGCCTTTGCCATC-3′ (SEQ ID NO:15) and 5′-AAACACTGTCTGCTGGTGGAGTTC-3′ (SEQ ID NO:16) for mouse Ifnb1. Reactions for Gapdh mRNA were performed concurrently on the same plate as those for the test mRNAs, and results were normalized by the corresponding amount of Gapdh mRNA.

### Soft Agar Colony Formation Assay

Each 6-well plate was coated with 1.5 mL of bottom agar (DMEM or RPMI 1640 containing 10% FBS and 0.5% Difco agar noble). Cells (5×10³ cells for B16-OVA and SCC7, 2.5×10³ cells for 4T1) were suspended in 1.5 mL of top agar (DMEM or RPMI 1640 containing 10% FBS and 0.35% Difco agar noble) into each well. Cells were cultured for approximately two weeks and replaced with fresh medium every three days. Colonies were stained using 0.005% crystal violet in 5% methanol and quantified using ImageJ software.

### Tumor Transplantation and Immunization

B16-OVA cells (2×10⁵) were subcutaneously transplanted into both back flanks of C57BL/6 mice. Tumor height and width were measured with a caliper every 2-3 days to calculate tumor volume (=width²×height×0.523). Mice were sacrificed when tumors reached maximum allowed size (15 mm in diameter) or when signs of ulceration were evident. Likewise, 1×10⁵ of SCC7 cells were subcutaneously transplanted into both back flanks of C3H/HeOu mice and 2.5×10⁵ of 4T1 cells were transplanted into both mammary fat pads of BALB/c mice. For 4T1 lung metastasis assay, lungs were tracheally injected with India ink 28 days after transplantation, and then destained in Fekete's solution to count tumor nodules.

For tumor vaccination experiments, C57BL/6 mice were immunized intradermally at the base of the tail with irradiated B16-OVA cells (100 Gy, 1×10⁶) 12 days prior to challenge with B16-OVA cells (one time vaccination, without any adjuvant). For immunization with EVs, C57BL/6 mice were inoculated with irradiated B16-OVA cells (100 Gy, 1×10⁶) at the base of the tail and EVs freshly isolated from culture supernatants of B16-OVA cells (6×10⁶) were injected every 3 days (days 0, 3, 6, and 9) into the same place until challenged with B16-OVA cells at day 12.

### Histopathology and Immunostaining of Tumors

Tumors were fixed with 4% paraformaldehyde in PBS, embedded in paraffin, sectioned with a microtome, and stained with hematoxylin-eosin according to standard procedures. Immunostaining of tumors was performed with frozen cryostat sections with PE-conjugated antibodies to CD45 (eBioscience, #12-0451-82).

### Measurement of OVA Specific Antibodies

Serum anti-OVA IgG concentrations were measured by enzyme-linked immunosorbent assay (ELISA). Briefly, half area 96-well plates (Corning) were coated with 5 mg/ml OVA protein (Worthington Biochemical, # LS003056) in PBS overnight at 4° C. Plates were washed and then blocked for 3 hours at room temperature with blocking buffer [1% BSA (bovine serum albumin) in PBS], followed by wash and incubation with serum samples tested at a 1:100 to 1:125,600 dilutions in blocking buffer overnight at 4° C. Plates were then washed and incubated with HRP-conjugated detecting antibody in blocking buffer at room temperature for 2 hours. Plates were washed and incubated with TMB substrate (KPL, #95059-286), and then read at 450 nm and 650 nm after stopping the development with 1 M phosphoric acid. Each ELISA plate contained a titration of a previously quantified serum to generate a standard curve. Anti-OVA IgG concentrations were determined from the lowest dilution of serum samples within a standard curve and reported as U/ml.

### Flow Cytometry

Flow cytometry was performed using a BD LSRFortessa and results were analyzed using FlowJo software (Treestar). Cell suspensions were incubated in mouse Fc block (anti CD16/CD32; BD Biosciences, #553142) prior to staining. Fluorochrome conjugated anti-mouse CD45 (clone 30F-11), CD3e (clone 145-2C11), CD8a (clone 53-6.7), Granzyme B (clone GB11), and IFNγ (clone XMG1.2) antibodies were used following the manufacturers protocol. K^{b}-SIINFEKL-tetramer was used for identifying OVA-specific CD8⁺ T cells. Propidium iodide (PI) was used to stain dead cells.

To analyze intracellular cytokine expression, cells were re-stimulated ex vivo with 10 mg/ml SIINFEKL peptide (AnaSpec, # AS-60193-1) for 5 hours in the presence of protein transport inhibitor (BD biosciences, #555029) for the last 4 hours. Intracellular cytokine staining was then performed using Fixation/Permeabilization Solution Kit (BD Biosciences, #554714).

### Ex Vivo Cytotoxicity Assay

EL4 cells were pulsed with 8 mg/ml SIINFEKL peptide or irrelevant peptides for 2 hours at 37° C., and then labeled with 0.25 mM or 2.5 mM CFSE (carboxyfluorescein succinimidyl ester; Thermo Fisher Scientific, # C34554) for minutes at 37° C., respectively. CFSE^{low} (SIINFEKL loaded target) and CSFE^{high} (irrelevant peptide control) EL4 cells were mixed at 1:1 ratio, and then co-cultured with CD8⁺ T cells isolated from splenocytes of C57BL/6 mice challenged (or not) with WT or LATS1/2 dKO B16-OVA cells at 8:1 effector to target cell ratio (E:T). CD8⁺ T cells were isolated using Easy Sep Mouse CD8a Positive Selection Kit (STEM CELL, #18753) from pooled splenocytes of 3-4 mice per group for each experiment. The frequencies of CFSE^{low} and CSFE^{high} EL4 cells in CFSE positive fraction were determined by flow cytometric analysis 18 hours after incubation and the percent of specific killing was calculated. Specific killing (%)=[1−“Sample ratio”/“Negative control ratio”]×100; “Sample ratio”=[CFSE^{low}(target)/CSFE^{high}(irrelevant)] value of each samples co-cultured with CD8⁺ T cells; “Negative control ratio”=[CFSE^{low}(target)/CSFE^{high}(irrelevant)] value of EL4 cells not cultured with CD8⁺ T cells.

### In Vitro Cross-Presentation Assay

For conditioned medium preparation, B16-OVA cells were seeded on culture plates and incubated in DMEM supplemented with 10% FBS for 24 hours at 37° C. to allow cell attachment. The cells were then washed with PBS, and culture medium was switched to DMEM without serum. After incubation for 48 hours, conditioned medium was collected and centrifuged at 2,000 g for 10 minutes at 4° C. to remove cell debris. The resulting supernatant was used for the experiment.

Naive CD8⁺ T cells were isolated from OVA-specific T cell receptor transgenic OT-I mice using Easy Sep Mouse CD8a Positive Selection Kit (STEM CELL) and labeled with 2 mM CFSE. Bone marrow derived dendritic cells (BMDCs) were generated by 6 days of GM-CSF (20 ng/ml; eBioscience, #14-8331-80) differentiation, and then incubated (or not) for 18 hours with conditioned medium (10% of the total volume) from WT or LATS1/2 dKO B16-OVA melanoma cells and pulsed with OVA protein (10 mg/ml) for the last 4 hours. The cells were washed and cultured with CFSE-labeled OT-ICD8⁺ T cells at 1:1 ratio for 3 days. OT-1 T cell proliferation was monitored by CFSE dilution using a flow cytometer and a division index was determined using FlowJo software (Treestar).

### Cytokine ELISA

IFNγ or IL-12 levels in culture supernatants were determined by ELISA. For ex vivo IFNγ production from lymph node cells, draining lymph nodes (inguinal lymph nodes) were isolated from C57BL/6 mice challenged (or not) with B16-OVA cells and cultured with OVA protein (100 mg/ml) for 3 days. For IL-12 production from BMDCs, BMDCs were generated by 6 days of GM-CSF (20 ng/ml) differentiation and stimulated (or not) for 18 hours with conditioned medium (10% of the total volume) or EVs isolated from culture supernatants of equal numbers of WT or LATS1/2 dKO B16-OVA cells (EVs from 2×10⁵ cells were used to stimulate 1×10⁶ BMDCs). Both cultures were done in RPMI 1640 supplemented with 10% FBS, penicillin (100 U/mL), and streptomycin (100 mg/ml) under an atmosphere of 5% CO₂ at 37° C., and then aliquots of cell culture supernatants were used for cytokine ELISA. For cell conditioned medium preparation, B16-OVA cells were seeded on culture plates and incubated in DMEM supplemented with 10% FBS for 24 hours at 37° C. to allow cell attachment. The cells were then washed with PBS, and culture medium was switched to DMEM without serum. After incubation for 48 hours, conditioned medium was collected and centrifuged at 2,000 g for 10 minutes at 4° C. to remove cell debris. The resulting supernatant was used for EV isolation, which is described in the “EV isolation and analysis” section.

IFNγ concentrations were determined using Mouse IFN-gamma DuoSet ELISA (R&D Systems, # DY485-05) according to a manufacturer's protocol. For IL-12 ELISA, half area 96-well plates were coated with capture antibody (Purified Rat Anti-Mouse IL-12 p40/p70; BD Biosciences, #551219) in PBS overnight at 4° C. Plates were washed and then blocked for 3 hours at room temperature with blocking buffer (1% BSA in PBS), followed by wash and incubation with culture supernatants overnight at 4° C. Plates were then washed and incubated with biotinylated detection antibody (Biotin Rat Anti-Mouse IL-12 (p40/p70; BD Biosciences, #554476) in blocking buffer at room temperature for 1 hour, followed by wash and incubation with streptavidin-HRP conjugate for 20 minutes. Plates were washed and incubated with TMB substrate (KPL, #95059-286), and then read at 450 nm and 650 nm after stopping the development with 1 M phosphoric acid. Concentrations were determined by comparison to a standard curve.

### EV Isolation and Analysis

B16-OVA cells were seeded in 150 mm culture plate and incubated in DMEM supplemented with 10% FBS for 24 hours at 37° C. to allow cell attachment. The cells were then washed with PBS twice, and culture medium was switched to 35 mL of DMEM without serum. After incubation for 48 hours, conditioned medium was collected and centrifuged at 2,000 g for 10 minutes at 4° C. to thoroughly remove cell debris. The resulting supernatant was then filtered through a 0.22 mm PVDF filter (Millip ore, # SLGV033RB) to remove cell debris and microvesicles (for the detergent treatment experiment, the resulting flow-through was treated with 1% Triton X-100 for 10 minutes at 4° C. prior to the ultracentrifugation). The flow-through was transferred into ultracentrifuge tubes (BECKMAN COULTER, #344058) and then ultracentrifuged in a Beckman SW32Ti rotor at 30,000 rpm for 90 minutes at 4° C. The resulting pellets were washed with 35 mL of PBS and then ultracentrifuged again at 30,000 rpm for 90 minutes at 4° C. The resulting EV pellets were re-suspended in PBS for experimental use. Protein concentrations of EVs were determined using Micro BCA Protein Assay Kit (Thermo, #23235). RNA in EVs was isolated using TRIzol reagents (Thermo, #15596026) according to the manufacturer's protocol and concentrations were determined using Agilent 2200 TapeStation (Agilent Technologies). For ribonuclease treatment, total RNA isolated from EVs was digested for 30 minutes at 37° C. with 100 mg/ml RNase A (Thermo, # EN0531) in a buffer comprising 10 mM Tris-HCl (pH 7.5), 5 mM EDTA, 300 mM NaCl. RNA was then resolved in agarose gels in non-denaturing conditions. Nanoparticle tracking analysis was performed using NanoSight NS300 system (Malvern Instruments, Ranch Cucamonga, Calif., USA) on isolated EVs diluted 5,000-fold with PBS for analysis.

### LC-MS/MS

EV samples were resolved in SDS-PAGE and the gels were cut into three regions, and then digested with try p sin. Extracted peptides were analyzed using a C₁₈ column and an EASY-nLC-1000 (Thermo Scientific) coupled to a hybrid quadrupole-orbitrap Q-Exactive mass spectrometer (Thermo Scientific). A data-dependent, top 50 method was utilized for analysis. The resulting RAW files were analyzed with Proteome Discoverer 1.4 and MASCOT. Results were filtered with 1% FDR at the protein level and exported to our in-house FileMakerPro database iSPEC and analyzed with Align!, which calculated intensity based absolute quantification (iBAQ) values (Schwanha\ddot{ }usser et al., 2011) that were used for subsequent analysis. The ratio of the iBAQ values for WT and LATS1/2 dKO EVs (DKO/WT ratio) in the individual experiments was calculated and scored according to the following criteria: score 2, >5-fold; score 1, 5- to 2-fold; score 0, 2- to 0.5-fold; corel, 0.5- to 0.2-fold; and score 2, <0.2-fold. We then added each scores from the individual experiments and set the threshold as a score of >3 for the top 100 most significantly increased proteins in LATS1/2 dKO EVs. Gene Ontology (GO) analysis was done using the PANTHER program (Mi et al., 2013). Heatmap s were generated using NetWalker (Komurov et al., 2012).

### Quantification and Statistical Analysis

### Statistical Analysis

Statistical analyses were performed using GraphPad Prism 5 software (GraphPad Software, Inc, La Jolla, Calif., USA). Statistical parameters and methods are reported in the FIGS. and the Figure Legends. A value of p<0.05 was considered statistically significant. Epidemiological data are obtained using the PrognoScan database (Mizuno et al., 2009). Association of gene expression with the survival of patients was evaluated using log-rank test and a value of p<0.05 was considered statistically significant.

### Results

### LATS1/2 Deletion Enhances Anchorage-Independent Growth In Vitro

To elucidate the role of the Hippo pathway in anti-tumor immunity, murine syngeneic tumor models of three different cancer types in three different host genetic backgrounds were used; B16-OVA melanoma (B16F10 melanoma expressing ovalbumin [OVA]) in C57BL/6 mice, SCC7 head and neck squamous cell carcinoma in C3H/HeOu mice, and 4T1 breast cancer in BALB/c mice. These syngeneic allograft models have been well characterized and extensively used to study reciprocal interactions between tumor cells and host anti-tumor immune responses (Dranoff, 2011; Lei et al., 2016). Deletion of LATS1/2 almost completely abolished YAP/TAZ regulation by the Hippo pathway, while deletion of other components had only a partial or minor effect on YAP/TAZ activity (Meng et al., 2015). Therefore, LATS1/2 was deleted in B16-OVA melanoma cells using CRISPR (clustered regularly interspaced short palindromic repeats)/Cas9 genome-editing technology (Ran et al., 2013). Multiple independent LATS1/2 double-knockout (dKO) clones were obtained as verified by the lack of protein expression of both LATS1 and LATS2 (FIG. 8A). Two different clones generated by two independent CRISPR guide sequences were used for this study. Because YAP is a direct substrate of LATS1/2, of which phosphorylation can be readily detected with a phospho-YAP antibody or by mobility shift on a phos-tag gel, we use YAP phosphorylation status as an indicator of LATS1/2 activity. We found that YAP phosphorylation levels were regulated in response to LATS1/2-activating signals in wild-type(WT) B16-OVA cells; however, loss of LATS1/2 abolished YAP phosphorylation (FIG. 8A). Phosphorylation of YAP/TAZ by LATS1/2 is known to promote YAP/TAZ cytoplasmic localization and inactivation (Zhao et al., 2007). Indeed, YAP/TAZ localized in the cytoplasmin response to filamentous actin disruption (which activates LATS1/2) in WT B16-OVA cells, yet YAP/TAZ remains localized in the nucleus in LATS1/2 dKO cells under the same condition (FIG. 8B). LATS1/2 inactivation or YAP/TAZ hyperactivation is known to promote cell growth (Zhao et al., 2008). Although LATS1/2 dKO B16-OVA cells showed identical growth on regular cell culture plates compared with WT cells (FIG. 15A), LATS1/2 dKO B16-OVA cells showed a significant increase in anchorage-independent growth in comparison to WT cells, in terms of both colony number and colony size (FIG. 8C). These observations indicate that the Hippo pathway is still operational in B16-OVA melanoma cells and, in addition, that LATS1/2 deficiency activates YAP/TAZ and can further potentiate anchorage-independent growth of B16-OVA cells in vitro.

LATS1/2 was also deleted in SCC7 squamous cell carcinoma cells and found that LATS1/2 deficiency almost completely blocked YAP phosphorylation (FIG. 15B) and YAP/TAZ cytoplasmic localization (FIG. 15C) in response to LATS1/2-activating signals. Notably, WT SCC7 cells showed high YAP phosphorylation and cytoplasmic localization of YAP/TAZ, even in the absence of LATS1/2-activating signals, suggesting high basal LATS1/2 activity in these cancer cells. Loss of LATS1/2 again increased anchorage-independent growth of SCC7 cells (FIGS. 8D and 15D). LATS1/2-dependent regulation of YAP phosphorylation (FIG. 15E), YAP/TAZ subcellular localization (Figure S1F), and anchorage-independent cell growth (FIG. 1E and S1G) were similarly observed in 4T1 breast cancer cells. Together, our data demonstrate that deletion of LATS1/2 in tumor cells promotes anchorage-independent tumor cell growth in vitro.

### Loss of LATS1/2 Inhibits Tumor Growth In Vivo

To investigate the role of the Hippo pathway in tumor growth in vivo, equal numbers of WT or LATS1/2 dKO B16-OVA cells were subcutaneously transplanted into the back flanks of C57BL/6 mice and monitored their growth. Unexpectedly, deletion of LATS1/2 in B16-OVA cells strongly inhibited tumor growth in vivo (FIGS. 9A and 9B). All mice died before day 22 in the WT B16-OVA-injected group, whereas injection with LATS1/2 dKO B16-OVA cells resulted in tumor-free survival in more than half of the mice (FIG. 9C). The growth-suppressive effect of LATS1/2 deletion was confirmed with an independent clone of LATS1/2 dKO B16-OVA cells (FIG. 16A). Next, we examined tumor growth of LATS1/2 dKO and WT SCC7 squamous cell carcinoma cells in syngeneic C3H/HeOu mice. All mice injected with the parental SCC7 cells showed aggressive tumor growth (FIGS. 9D and 16B), and 100% died before day 21 (FIG. 9E). In contrast, none of the mice injected with LATS1/2 dKO SCC7 cells developed tumors, and all survived tumor free. In a 4T1 orthotopic allograft model, 4T1 breast cancer cells grow into solid tumors and can readily metastasize to the lung, liver, and brain when transplanted into the mammary fat pads of syngeneic BALB/c mice. Consistently, the parental 4T1 cells developed tumors and metastasized to the lung in BALB/c mice (FIGS. 9F, 9G, and 16C). On the other hand, LATS1/2 dKO 4T1 cells developed little tumors and had no metastasis when allografted in BALB/c mice. Thus, collectively, these observations indicate that loss of LATS1/2 in tumor cells dramatically inhibits tumor growth in vivo in multiple types of cancer in different host backgrounds. Based on the current dogma, these results are totally unexpected, as LATS1/2 kinases supposedly function as tumor suppressors.

### LATS1/2 Deletion Enhances Immunogenicity of Tumor Cells

Since LATS1/2 deletion exerts completely opposite effects on tumor cell growth in vitro and in vivo (FIGS. 8-9), it was hypothesized that host factors may contribute to the apparent discrepancy between in vitro and in vivo phenotypes of LATS1/2 dKO tumor cells. Therefore, we examined the histopathology of tumors from allografted mice. Massive infiltration of inflammatory cells in LATS1/2 dKO B16-OVA melanomas (FIG. 10A), as well as in LATS1/2 dKO 4T1 breast cancers (FIG. 17A), was found, which were confirmed by staining with the pan-leukocyte marker CD45 (FIGS. 10B and 17B). These observations prompted the hypothesis that immune cells infiltrate, and thereby eliminate, LATS1/2 dKO tumor cells. Both innate and adaptive immune responses work together to constitute host anti-tumor immunity, but the adaptive immune system plays a pivotal role in mediating robust and highly specific immune responses against tumors (Gajewski et al., 2013). Therefore, host adaptive immune responses against tumor cells was examined. A B16-OVA melanoma model was used because B16-OVA melanoma cells express a non-secreted form of chicken OVA as a surrogate tumor antigen that can be conveniently used to follow immune responses directed against the OVA antigen. In addition, B16-OVA has been extensively used to study cancer immunity, and many genetically altered syngeneic mouse C57BL/6 strains are available.

Although WT and LATS1/2 dKO B16-OVA cells showed identical expression of OVA (FIG. 10C), we detected significantly higher levels of serum anti-OVA antibody in mice injected with LATS1/2 dKO B16-OVA cells (FIG. 10D), suggesting an enhanced tumor-specific humoral immune response in the LATS1/2 dKO B16-OVA-injected mice. Next, cellular immune responses were examined. CD8⁺ T cells isolated from the spleens of LATS1/2 dKO B16-OVA-injected mice produced multiple effector cytokines (FIGS. 10E and 17C), indicative of T cell activation. Significantly higher CD8⁺ T cell crosspriming was observed when mice were injected with LATS1/2 dKO B16-OVA cells (FIGS. 10F and 17D), and in addition, lymph node cells isolated from draining lymph nodes of LATS1/2 dKO B16-OVA injected mice showed a remarkably higher OVA-specific T cell response than did lymph node cells isolated from the parental B16-OVA-injected mice, as measured by interferon (IFN)g production (FIG. 10G). These observations suggest that tumor specific cellular immune responses, particularly CD8⁺ T cell responses, are induced in mice injected with LATS1/2 Dko B16-OVA cells. Indeed, CD8⁺ T cells in LATS1/2 dKO B16-OVA-injected mice possessed OVA-specific cytotoxic activity ex vivo (FIGS. 3H and S3E) and infiltrated tumors in vivo (FIGS. 10i and 17F). Together, the aforementioned data demonstrate that LATS1/2 deletion in tumor cells stimulate tumor-specific humoral and cellular immune responses, leading to the establishment of robust anti-tumor immunity.

### LATS1/2 Deficiency Enhances Tumor Vaccine Efficacy Via Adaptive Immunity

Given that LATS1/2 deletion in tumor cells enhances host antitumor immune responses, it was hypothesized that LATS1/2-null tumor cells, by stimulating anti-tumor immunity, may protect the host from challenge with the corresponding LATS1/2 WT tumor cells. To test this, we performed two sets of experiments: co-injection of LATS1/2 dKO and WT tumor cells into each side of the same mouse (FIG. 11A) or immunization of mice with LATS1/2 dKO tumor cells prior to LATS1/2 WT tumor cell injection (FIGS. 11B-C). Strikingly, co-injection of LATS1/2 dKO B16-OVA cells significantly suppressed tumor growth of the co-injected WT B16-OVA cells (FIG. 11A). Moreover, immunization of mice with irradiated LATS1/2 dKO B16-OVA cells, which were viable but unable to proliferate, strongly inhibited the corresponding LATS1/2 WT tumor growth, whereas immunization with irradiated parental B16-OVA cells produced a much weaker effect (FIG. 11B). Notably, in this experimental setting a single dose of tumor vaccination with irradiated WT B16-OVA cells was not sufficient to extend survival (FIG. 11C). In contrast, a single dose of tumor vaccination with irradiated LATS1/2 Dko B16-OVA cells showed a significant delay in tumor growth and prolonged survival. Approximately 25% of the mice immunized with irradiated LATS1/2 dKO B16-OVA cells were tumor free when challenged with WT B16-OVA cells. The aforementioned observations suggest that LATS1/2 deficiency renders B16-OVA cells highly immunogenic and improves tumor vaccine efficacy. Enhanced anti-tumor immunity was confirmed by LATS1/2 deletion with a different syngeneic model. C3H/HeOu mice having rejected LATS1/2 dKO SCC7 cells were resistant to rechallenge with the parental SCC7 cells (FIG. 11D), indicating that these animals have established immunological memory against the given tumor cells.

Next, it was tested whether adaptive immunity is required for tumor suppression by LATS1/2 deletion. WT or LATS1/2 dKO B16-OVA cells were subcutaneously transplanted into RAG-1 (recombination activating gene 1) knockout (KO) mice that are immune-compromised due to the lack of mature T and B cells. LATS1/2 dKO B16-OVA tumor cells grew similarly to WT cells (FIG. 11E) and showed comparable mortality (FIG. 11F) in the absence of an adaptive immune system. Consistently, co-injection of LATS1/2 dKO B16-OVA cells failed to inhibit the corresponding LATS1/2 WT tumor growth in RAG-1 KO mice (FIG. 11G). Based on the aforementioned data, LATS1/2 deletion in tumor cells enhances immunogenicity and provokes an adaptive immune response to eliminate tumor cells.

### YAP or TAZ Overexpression in Tumor Cells Suppresses Tumor Growth In Vivo

YAP and TAZ are the most characterized downstream effectors of the Hippo pathway. LATS1/2 cells directly phosphorylate YAP/TAZ on multiple serine residues, leading to cytoplasmic retention, degradation, and thereby inactivation of YAP/TAZ. Because high YAP/TAZ activation was observed in LATS1/2 dKO B16-OVA tumors in vivo (FIGS. 18A-B), it was examined whether YAP/TAZ hyperactivation phenocopies the effect of LATS1/2 deletion in tumor growth. To this end, B16-OVA cells stably overexpressing YAP(5SA) or TAZ(4SA) were generated. YAP(5SA) and TAZ(4SA) are active mutants of YAP/TAZ, with the LATS1/2 phosphorylation sites mutated to alanine, thereby unresponsive to inhibition by LATS1/2. Notably, a mutual inhibition between YAP and TAZ protein abundance in YAP(5SA)- or TAZ(4SA)-overexpressing B16-OVA cells (FIG. 18C) was observed, consistent with the previously described negative-feedback response (Moroishi et al., 2015b). YAP(5SA)- or TAZ(18A)-overexpressing B16-OVA cells showed increased anchorage-independent growth potential in comparison to the control cells in vitro (FIG. 18D), while their tumor growth in vivo was significantly delayed (FIG. 18E). Next it was investigated whether the effect of YAP(5SA) requires its transcriptional activity. YAP mainly binds to the TEA domain (TEAD) family of transcription factors (TEAD1-4) to induce gene expression, and er94 in YAP is required for TEAD binding (Zhao et al., 2008). As expected, mutating Ser94 abolished the ability of YAP(5SA) to induce target gene transcription (FIGS. 18F-G). Importantly, the TEAD-binding-defective YAP(5SA/S94A) was unable to suppress B16-OVA tumor growth (FIG. 18H), suggesting that tumor suppression by YAP requires TEAD-dependent transcription. Together, these observations indicate that hyperactivation of YAP and TAZ significantly, though may not entirely, contributes to the in vivo tumor growth suppression by LATS1/2 deletion through a mechanism requiring TEAD-mediated transcription.

Extracellular Vesicles Released from LATS1/2-Null Tumor Cells Stimulate Immune Responses

Next it was explored how LATS1/2 deficiency in tumors stimulates host anti-tumor immune responses. Because we observed a preeminent CD8⁺ T cell cross-priming in mice injected with LATS1/2 dKO B16-OVA cells (FIG. 10F), it was hypothesized that LATS1/2 dKO B16-OVA cells stimulate cross-presentation by antigen-presenting cells. To test this, the effects of LATS1/2 dKO B16-OVA cells on MHC (major histocompatibility complex) class I-restricted cross-presentation was examined using bone marrow-derived dendritic cells (BMDCs) as antigen-presenting cells. It was found that pre-treatment of BMDCs with conditioned medium from LATS1/2 dKO B16-OVA cells significantly augmented antigen cross-presentation in comparison to WT conditioned medium (FIG. 12A), and consistent with this, LATS1/2 dKO conditioned medium enhanced BMDC activation, as assessed by interleukin (IL)-12 production (FIG. 12B). These results imply that factors released from LATS1/2 dKO B16-OVA cells activate BMDCs and thereby enhance antigen cross-presentation. Recent studies have revealed the emerging roles of extracellular vesicles (EVs) in immune regulation, both in an immunosuppressive and in an immunostimulatory manner (Robbins and Morelli, 2014). Therefore, it was investigated whether Evs secreted from LATS1/2 dKO B16-OVA cells are capable of stimulating immune responses. EVs were isolated from culture supernatants of WT or LATS1/2 dKO B16-OVA melanoma cells by ultracentrifugation (FIG. 19A) and it was found that EVs from LATS1/2 dKO B16-OVA cells were more potent than EVs from WT B16-OVA cells in activating BMDCs, as assessed by IL-12 production in vitro (FIG. 12B). To discriminate EVs from extracellular non-membranous particles that may be enriched by ultracentrifugation, we treated the culture supernatants with detergent (Triton X-100) prior to EV purification. The detergent-treated EV pellets failed to activate BMDCs (FIG. 19B). More importantly, LATS1/2 dKO EVs imp roved the tumor vaccine efficacy of irradiated WT B16-OVA cells and conferred a strong immunity against tumor challenge in vivo (FIG. 5C). Thus, these results show that EVs released from LATS1/2-deficient tumor cells induce immune responses and are sufficient to render LATS1/2-adequate tumor cells highly immunogenic.

### LATS1/2-Deficient Tumor Cells Secrete Nucleic-Acid-Rich EVs

To elucidate the mechanistic basis for immunostimulatory effects of LATS1/2 dKO EVs, the nature of EVs released from WT or LATS1/2 dKO B16-OVA cells was characterized. It was found that LATS1/2 dKO B16-OVA cells produced slightly more EVs compared with WT cells, as assessed by nanoparticle tracking analysis (FIGS. 12D and 19A) as well as by protein quantification (FIG. 12E). The proteome of EVs was analyzed using quantitative mass spectrometry. A total of 1,772 proteins were identified in EVs, which showed enrichment of previously reported exosomal and microvesicle cargo proteins (FIG. 20B), supporting the quality of EV purification. Most of the protein expression was almost identical between WT and LATS1/2 dKO EVs, but a subset of proteins were highly elevated in LATS1/2 dKO EVs (FIG. 20C). Among the top increased proteins in LATS1/2 dKO EVs were those involved in RNA and nucleic acid binding (FIG. 20D). These observations prompted us to hypothesize that LATS1/2-null tumor EVs contain higher amounts of nucleic acids, which are previously reported contents of EVs (Yanez-Mo et al., 2015) and are also well known immunostimulators (Junt and Barchet, 2015). Because RNA is the most abundant nucleic acid in EVs (Robbins and Morelli, 2014), we characterized total RNA isolated from EVs. RNA contents in LATS1/2 dKO or YAP(5SA)-overexpressing EVs were dramatically increased in comparison to WT EVs (FIGS. 12F and 20E). The RNAs in EVs were sensitive to single-strand-specific ribonuclease treatment (FIG. 20F). Taken together, our observations suggest a model in which LATS1/2-deficient tumor cells secrete higher amounts of nucleic-acid-rich EVs that may contribute to the potent immunostimulatory effects.

EVs from LATS1/2 dKO Tumor Cells Stimulate the Toll-Like Receptors-Type I Interferon Pathway

To test the hypothesis that nucleic-acid-rich EVs released from LATS1/2-null tumors stimulate host anti-tumor immunity, it was examined whether alterations in the host nucleic-acid-sensing pathways imp air the tumor-protective effects of LATS1/2 deletion in vivo. Both microbial (non-self) and self nucleic acids can be recognized by distinct families of pattern recognition receptors, including endosomal Toll-like receptors (TLRs) and cytosolic non-TLR sensors (FIG. 21A). Activation of these pathway s results in the production of inflammatory cytokines as well as type I IFN, which stimulates innate and adaptive immunity (Junt and Barchet, 2015). WT or LATS1/2 dKO B16-OVA cells were subcutaneously transplanted into C57BL/6 mice deficient in the following key molecules in the endogenous nucleic-acid-sensing pathways: MYD88 (myeloid differentiation primary response 88) and TRIF (TIR-domain-containing adaptor-inducing interferon-b, also known as TICAM1), two adaptor proteins required for TLR signaling STING (stimulator of interferon genes, also known as TMEM173), an adaptor protein required for the cGAS (cyclic GMP-AMP synthase, also known as MB21D 1) cytoplasmic DNA-sensing pathway; and caspase-1 (also known as CASP 1), an effector protein involved in IL-1b maturation under the AIM2 (absent in melanoma 2) cytoplasmic DNA-sensing pathway. We found that deletion of MYD88 largely (FIGS. 13A and 21B), and TRIF deficiency considerably (FIGS. 13B and 21C), attenuated the tumor-suppressive effects of LATS1/2 deletion, as assessed by tumor mortality. In contrast, deletion of STING (FIG. 13C) or caspase-1 (FIG. 13D) in recipient mice had no effect on tumor protection by LATS1/2 deficiency, suggesting that the TLRs-MYD88/TRIF nucleic acid sensing pathway, not the cytoplasmic DNA-sensing pathway, is required for immunostimulatory effects of LATS1/2 deletion.

Distinct types of TLRs utilize MYD88 or TRIF as adaptor proteins and specifically respond to a wide range of ligands on the cell surface, as well as in the endosome (FIG. 21A). The endosomal TLRs are intrinsically capable of detecting nucleic acids. It was further investigated which TLR is required for tumor suppression by LATS1/2 loss. Whereas LATS1/2 deletion in tumors still protected mice from tumor challenge in TLR4 (which senses bacterial lipopolysaccharides) KO mice (FIG. 13E), deletion of TLR7 (which senses single-stranded RNA) (FIG. 13F) or TLR9 (which senses double-stranded DNA) (FIG. 13G) in recipient mice partially, but significantly, impaired tumor protection by LATS1/2 loss. Thus, our data suggest that multiple TLRs, and probably not a single TLR, cooperatively sense the nucleic acid-rich EVs secreted from LATS1/2-null tumors and trigger immune responses through the MYD88/TRIF signaling pathway.

Activation of TLRs-MYD88/TRIF signaling results in pro-inflammatory cytokine production as well as type I IFN (in particular, IFNa and IFNb) production, which stimulates anti-tumor immune responses (Figure S7A). Particularly, type I IFN plays a central role in anti-tumor immunity by promoting dendritic cell maturation, antigen cross-presentation, and CD8⁺ T cell clonal expansion (Fuertes et al., 2013). Therefore, it was examined whether host type I IFN signaling is required for establishing host anti-tumor immunity induced by LATS1/2 dKO tumor cells. To test this, we subcutaneously transplanted WT or LATS1/2 dKO B16-OVA cells into IFNAR1 (interferon a and b receptor subunit 1) KO mice that are deficient in a functional type I IFN receptor. We found that loss of host type I IFN signaling largely obliterated the protective role of LATS1/2 deletion in tumor growth (FIG. 13H) as well as tumor mortality (FIG. 13I). Thus, collectively, the data provide in vivo evidence supporting a model in which EVs secreted from LATS1/2-deficient tumor cells stimulate the host TLRs-MYD88/TRIF nucleic-acid-sensing pathways to incite type I IFN signaling and establish robust anti-tumor immunity.

### Discussion

In this study, it was demonstrate that LATS1/2 deletion unmasks a malignant cell's immunogenic potential and restrains tumor growth due to the induction of anti-tumor immune responses. The effects of LATS1/2 deletion on tumor growth are striking insofar as LATS1/2 dKO completely abolishes the tumor growth potential of SCC7 and dramatically reduces tumor growth and the metastasis of B16 and 4T1 cells. LATS1/2-null B16 melanomas secrete nucleic-acid-rich EVs that stimulate the host TLRs-MYD88/TRIF-IFN pathway s to induce anti-tumor immunity and the eventual elimination of tumor cells (FIG. 7). LATS1/2 deletion similarly stimulates host immune responses in both SCC7 and 4T1 syngeneic models (FIGS. 11D, 17A, and 17B), though the involvement of EVs has only been examined in the B16 model in this study.

### Dual Functions of LATS1/2 in Cancer

It is generally accepted that the Hippo pathway is a tumor suppressor that inhibits proliferation and survival of normal cells, preventing tumorigenesis (Harvey et al., 2013; Moroishi et al., 2015a; Wang et al., 2014), yet a few studies did suggest an oncogenic role of the Hippo pathway in certain contexts (Barry et al., 2013; Cottini et al., 2014). We have analyzed human epidemiological data using the PrognoScan database (Mizuno et al., 2009) to find any correlation between LATS1/2 mRNA expression levels and patient outcome in different types of human cancer. Among 107 epidemiological datasets available, 26 studies show significant (p<0.05) correlation between LATS2 mRNA levels and patient outcome, which includes 17 studies showing better patient survival with low LATS2 expression. Moreover, 12 studies show significant correlation between LATS1 mRNA levels and patient outcome, which includes 5 studies showing better patient survival with low LATS1 expression. In addition, low YAP expression predicted worse patient survival in human colorectal cancer (Barry et al., 2013) and multiple myeloma (Cottini et al., 2014). Therefore, although YAP/TAZ hyperactivation is frequently observed in human cancers (Harvey et al., 2013; Moroishi et al., 2015a), the precise role of the Hippo pathway in human cancer might be context dependent. In this study, we show that deletion of LATS1/2 in tumor cells strongly suppresses tumor growth in vivo. On the surface, the present data cannot be easily reconciled with the tumor suppressor model of LATS1/2 in the Hippo field.

The following model is proposed: LATS1/2 suppress tumor initiation as well as inhibit immunogenicity. These two activities are important for the physiological role of LATS1/2 in maintaining tissue homeostasis. LATS1/2 normally provide growth inhibitory signals to the cells; therefore, they function cell autonomously to limit tissue overgrowth. It is also proposed that LATS1/2 suppresses immunogenicity, serving as a built-in mechanism to prevent overgrowth of undesirable cells at the wrong places in the organism. For example, inactivation of LATS1/2 is needed to promote cell proliferation during wound healing and tissue regeneration. However, cells with imp aired LATS1/2 activity may over-proliferate and migrate to the wrong place. Such undesirable cells should be eliminated to maintain tissue homeostasis and integrity. This can be achieved because inactivation of LATS1/2 in these cells can induce a strong immune response. Therefore, the immunosuppressive function of LATS1/2 is consistent with its physiological roles in tissue homeostasis.

In the established tumor cell lines of B16, SCC7, and 4T1, YAP and TAZ are not constitutively active. In fact, YAP and TAZ are readily regulated (in B16 and 4T1 cells) or even largely inactive (in SCC7 cells). Therefore, the tumorigenicity of these cancer cell lines is independent of the Hippo pathway. Nevertheless, deletion of LATS1/2 causes a moderate increase of anchorage-independent growth of these tumor cells in vitro, consistent with the growth inhibitory effect of LATS1/2. However, the enhanced immunogenicity unmasked by the LATS1/2 deletion in these cells can induce strong immune responses and overwhelm any growth advantage that might be gained due to LATS1/2 deletion, leading to strong inhibition of tumor growth in the immune-competent mice. The dual functions of LATS1/2 in sup pressing cell growth and immunogenicity can explain previous observations along with the present data.

### Hippo Pathway in Inflammation and Tumor Immunogenicity

The present results indicate that inactivation of the Hippo pathway in tumor cells induces host inflammatory responses. Interestingly, recent studies revealed that the Hippo pathway can respond to (Nowell et al., 2016; Taniguchi et al., 2015) and mediate (Liu et al., 2016) inflammatory signals. This study, together with these recent findings, suggests a reciprocal interaction between the Hippo pathway and inflammatory responses. LATS1/2-deficient tumor-derived EVs contain higher amounts of nucleic acids, which stimulate the host TLRs-MYD88/TRIF nucleic-acid sensing pathway s, provoking a type I IFN response to establish robust anti-tumor immunity. Recent studies indicate that tumor cells themselves can produce type I IFN in response to chemotherapy, thus enhancing anti-tumor immune responses (Chiappinelli et al., 2015; Sistigu et al., 2014). Because WT and LATS1/2 dKO B16-OVA cells showed similar expression levels of type I IFN genes such as Ifna4 and Ifnb1 (Figure S7D), it is less likely that type I IFN secreted from LATS1/2-null tumor cells is the main mechanism conferring the anti-tumor immunity evoked by LATS1/2 deletion. Given that nucleic-acid-rich EVs from LATS1/2-deficient tumors can stimulate dendritic cells in vitro (FIG. 12B), and that the host TLRs-MYD88/TRIF nucleic acid sensing pathways are required for immunostimulatory effects of LATS1/2 deletion in vivo (FIG. 13), host immune cells in the tumor microenvironment may be the major source of type I IFN (Fuertes et al., 2013).

A series of unbiased Hippo pathway interactome studies have linked endosomal compartments to the Hippo pathway (Moya and Halder, 2014). It is possible that the Hippo pathway may regulate endocytic trafficking and, therefore, regulate EV biogenesis. Little is known about the signaling mechanisms involved in EV biogenesis and incorporation of proteins or nucleic acids into EVs. Given the known effect of YAP on global microRNA (miRNA) biogenesis (Mori et al., 2014) and the functional importance of miRNA in EVs (Yanez-Mo et al., 2015), one may speculate that the effect of YAP/TAZ on miRNA biogenesis may increase immunogenicity of LATS1/2-null cells. However, TEAD mediated transcription is required for tumor suppression by YAP (FIG. 18H), whereas TEAD-dependent transcription is dispensable for YAP-influenced miRNA biogenesis (Mori et al., 2014). Moreover, LATS1/2 dKO cells do not increase miRNA contents in EVs (FIG. 20E). Therefore, YAP/TAZ hyperactivation suppresses tumor growth in vivo via a transcription-dependent, but miRNA-biogenesis-independent, mechanism. The tumor growth suppression by YAP/TAZ overexpression (FIG. 20E) is not as strong as that of LATS1/2 deletion (FIG. 9A), suggesting that LATS1/2 may have additional targets to suppress immune responses. Recent studies revealed new LATS1/2 substrates in spindle orientation (Dewey et al., 2015; Keder et al., 2015). Because aneuploidy plays a role in tumor immunogenicity (Senovilla et al., 2012), these new LATS1/2 substrates in spindle regulation could contribute to immunosuppression.

### Targeting the Hippo Pathway for Cancer Immunotherapy

Recent advances in cancer immunotherapy have provided new therapeutic approaches for cancer, and several immune checkpoint inhibitors indeed show impressive effects in the clinic (Sharma and Allison, 2015). However, individual immune response to cancer immunotherapy often relies on tumor immunogenicity that varies extensively between different cancer types and different individuals; therefore, immune checkpoint inhibitors may not work in cases where tumor immunogenicity is intrinsically limited (Pico de Coana et al., 2015). The present study revealed that inactivation of LATS1/2 in tumor cells increases tumor immunogenicity and enhances tumor vaccine efficacy. Therefore, it is speculated that inhibiting LATS1/2 may enhance anti-tumor immune response and, therefore, would be an attractive approach to treat cancer. Furthermore, LATS1/2 inhibition to improve immunogenicity of tumor cells may enhance immune checkpoint inhibitor efficacy. Thus, a combination of LATS1/2 inhibitors and immune checkpoint inhibitors would be a novel and exciting therapeutic app roach for poorly immunogenic cancers, especially in cases where malignancy is driven by oncogenic alterations that leave the Hippo signaling pathway intact. It is noteworthy that germline or somatic mutations affecting the core components of the Hippo pathway are uncommon in human cancers (Harvey et al., 2013; Moroishi et al., 2015a). Therefore, inhibition of LATS1/2 may enhance tumor immunity in most cancer types. However, the caveat remains that the immune system of mice is considerably different from that of humans, and whether our findings in mice can directly be translated to humans remains to be determined. Moreover, the effect of LATS1/2 inhibition as an intervention for established tumors needs to be explored. Nevertheless, future studies expanding the therapeutic potentials of the Hippo pathway will have important clinical implications.

### Example 3

Recent advances in cancer treatment have improved the survival and quality of life for patients, especially those who are in the early clinical stages (Steeg 2016). However, prolonged survival is still unachievable in most patients with advanced cancer that have distal metastases. In particular, metastases of primary tumors contribute to 90% of patient deaths (Lambert et al., 2017). The lungs are the second most frequent site of metastases from extra-thoracic malignancies (Mohammed et al., 2011). Cancer in the lungs can create an immunosuppressive and angiogenic microenvironment (Kitamura et al., 2015; Ostrand-Rosenberg and Fenselau, 2018). Recent studies suggest that immunotherapy with checkpoint inhibitors can overcome the immunosuppressive networks to prevent, and in some cases, to eradicate lung metastases (Sharma and Allison, 2015, Wolchok et al., 2013). However, checkpoint inhibitors achieve a progression-free survival in only 10-30% of patients with metastasis (Wolchok et al., 2017). Hence, there are strong demands for new immunotherapeutic approaches to improve survival rates of metastatic cancer patients.

A synthetic TLR7 agonist, imiquimod, has already been approved for human use and shows favorable clinical efficacy in patients with dermatological tumors, including basal cell carcinoma and actinic keratosis (Geisse et al., 2004). However, the drugs applications are limited to topical use because of immunotoxicity induced by systemic administration (Savage et al., 1996; Engel et al., 2011). To improve the pharmacokinetics and reduce severe immune adverse effects, our laboratory developed 1V270, a small molecule TLR7-specific ligand (1V136, SM360320) conjugated to a phospholipid moiety (Chan et al., 3009; Wu et al., 2014). Herein below it is demonstrated that intratumoral administration of 1V270 induces tumor-specific adaptive immune responses and inhibits primary tumor growth in murine syngeneic models of head and neck cancer and melanoma (Hayashi et al., 2011; Sato-Kaneko et al., 2017). The local 1V270 treatment activates tumor-associated macrophages and converts an immune-suppressive tumor microenvironment to a tumoricidal environment without causing systemic cytokine induction (Sato-Kaneko et al., 2017). Subsequently, 1V270 therapy induced tumor-specific adaptive immune responses that suppressed tumor growth in uninjected tumors. Reports by others have demonstrated that the use of local or systemic TLR 7 agonists, alone or as vaccine adjuvants, can induce tumor-specific immune responses and reduce the growth of colon, renal and mammary carcinomas (Wange et al., 2010; Koga-Yamakawa et al., 2013).

In contrast to the therapeutic advantages of using TLR 7 agonists on the innate immune cells in the tumor microenvironment, some recent reports indicate that TLR7 signaling pathway may promote tumor growth in primary lung carcinoma (Cherfils-Vicini et al., 2010; Chatterjee et al., 2014). This phenomenon is attributable to increased recruitment of myeloid-derived suppressor cells (MDSCs) to the tumor following TLR7 therapy (Chatterjee et al., 2014; Ochi et al., 2012). Thus, TLR7 therapy can be a double-edged sword depending on the type of tumor, the levels of receptor expression, and infiltration of suppressor cells in the tumor microenvironment (Dajon et al., 2015).

In this study, the effects of 1V270 therapy on metastatic lung tumors was evaluated. Since metastatic lung tumors are not readily accessible to intratumoral drug delivery, parenteral drug administration was analyzed. Therapeutic effects of 1V270 were evaluated in three murine syngeneic tumor models, 4T1 breast cancer, B16 melanoma, and Lewis lung carcinoma (LLC). The results showed that a single systemic dose of 1V270 reduced tumor lung colonization in all three models tested. Systemic 1V270 therapy activated local innate immune cells, including natural killer (NK) cells and antigen presenting dendritic cells. T cell receptor (TCR) repertoire analyses revealed that 1V270 therapy induced tumor-specific oligoclonal cytotoxic T cells, that were shared by different mice, and that suppressed the growth of metastatic tumors. Anti-metastatic effects of 1V270 were also achieved by intranasal drug administration. These document that both local and systemic therapy with a phospholipid-conjugated TLR 7 agonist can safely induce tumor-specific cytotoxic T cell responses in pulmonary metastatic cancer, that peripheral T cell repertoire analysis may be used to monitor the effects of therapy.

Observations support the concept that an immunomodulatory drug and TCR repertoire analysis can be applied for monitoring prognosis of metastatic lung cancer.

### Materials and Methods

### Animals, and Reagents

Wild-type female BALB/c mice, C57BL/6 and BG-albino mice were purchased from Jackson Laboratory (Bar Harbor, Mass.). The studies involving animals were carried out in strict accordance with the recommendations in the Guide for the Care and Use of Laboratory Animals of the National Institutes of Health. A small molecule TLR7 ligand (1V136, SM360320) and phospholipid-conjugated TLR7 agonist, 1V270, were synthesized in our laboratory as described previously and was formulated in 20% hydroxypropylbeta-cyclodextrin (Chan et al., 2009). Endotoxin levels of these drugs and other reagents were determined by Endosafe® (Charles River Laboratory, Wilmington, Mass.) and were less than 15 EU/mg

### Lung Metastasis Models

The 4T1 (mouse breast cancer cell line), B16 melanoma cell line, and LLC cell line and BALB/3T3 fibroblast (Clone A31, CLL163) were obtained from American Type Culture Collection (Rockville, Md., USA). The cells were tested for murine pathogens and were confirmed negative prior to inoculation in mice. A GLF-expressing 4T1, B16 and LLC cells were prepared as described previously (Godebu et al., 2014). Two metastatic 4T1 models, spontaneous and IV metastasis models, were used in this study.

### In Vivo Imaging Study of Lung Tumor Growth

GFP and luciferase (GLF)-expressing tumor cells (2×10⁴ of 4T1-GLF, 5×10⁵ of B16-GLF, and 1×10⁶ of LLC-GLF) were i.v. injected into BALB/c mice for 4T1 models, BG-albino or wild-type C57BU6 for B16 melanoma or LLC models). To generate bioluminescence signals, D-luciferin (3 mg/100 μL/mouse) was injected i.p. 12-15 minutes prior to the image acquisition. Image data were acquired by 1 Ss exposure using the IVIS Spectrum and analyzed using the Living Image software, version 4.5.2 (Perkin Elmer, Waltham, Mass.). We confirmed that the tumor signals in the lungs at day 10 correlated with the number of lung metastasis determined on day 21, as well as the overall survival (Supplemental figures 10).

### Flow Cytometric Analysis

The cells were labeled by incubating with cocktails of antibodies at 4° C. for 30 minutes (Table 1) to identify various cell types.

TABLE 1
Antibodies used in flow cytometry analysis

| Antibody | Color | Cat# | Clone | Host/Isotype | Vendor |
|---|---|---|---|---|---|
| CD3 | PE/Cy7 | 552774 | 145-2c11 | Armenian | BD |
| | | | | Hamster IgG1, K | Biosciences |
| CD4 | APC | 17-0042 | RM4-5 | Rat IgG2a, K | eBioscience |
| CD8a | e450 | 48-0081 | 53-6.7 | Rat IgG2a, K | eBioscience |
| CD11b | FITC | 11-0112 | M1/70 | Rat IgG2b, K | eBioscience |
| CD11b | e450 | 48-0112 | M1/7C | Rat IgG2b, K | eBioscience |
| CD11c | APC/Cy7 | 117324 | N418 | Armenian | Biolegend |
| | | | | Hamster IgG | |
| CD44 | APC/Cy7 | 103028 | IM7 | Rat IgG2b, K | Biolegend |
| CD45 | PE/Cy7 | 103114 | 30-F11 | Rat IgG2b, K | Biolegend |
| CD49b | PE | 553858 | DX5 | Rat Lewis IgM, K | BD |
| | | | | | Biosciences |
| CD62L | FITC | 11-0621 | MEL-14 | Rat IgG2a, K | eBioscience |
| CD80 | FITC | 104706 | 16-10A1 | ArmenianHamster | Biolegend |
| | | | | IgG | |
| CD86 | PE | 12-0862 | GL1 | Rat IgG2a, K | eBioscience |
| CD279(PD-1) | PE | 135205 | 29F.1A12 | Rat IgG2a, K | Biolegend |
| CD335(NKP40) | FITC | 560756 | 29A1.4 | Rat IgG2a, K | BD |
| | | | | | Biosciences |
| F4/80 | FITC | 11-4801 | BM8 | Rat IgG2a, K | eBioscience |
| LY6C | Pacific | 128014 | HK1.4 | Rat IgG2c, K | Biolegend |
| | blue | | | | |
| LG6G | APC | 127614 | 1A8 | Rat IgG2a, K | Biolegend |
| MHC Class2(1- | APC | 17-5323 | AMS-32.1 | Mouse IgG2b, K | eBioscience |
| ad) | | | | | |
| Granzyme B | FITC | 515403 | GB11 | Mouse IgG1, K | BD |
| | | | | | Biosciences |
| IFNr | APC | 17-7311 | XMG1.2 | Rat IgG1, K | eBioscience |

### NK Cells and CD8⁺ Cell Depletion In Vivo

Anti-asialo GM1 rabbit polyclonal antibody (Waka, Richmond, Va.) or rabbit 1 gG polyclonal antibody (Millipore, Temecula, Calif.) was used for NK cell depletion. Mouse anti-CDS (clone 2.43), and isotype control Ab (clone LFA-2) were used for CD8⁺ cell depletion. We confirmed over 90% depletion of NK cells and CD8⁺ cells using flow cytometry (FIGS. 28 and 33).

### Ex Vivo Tumor-Specific Cytotoxicity Study

Tumor-specific cytotoxicity was examined using 4T1 cells as target cells and BALB/3T3 cells as irrelevant cells. BALB/c mice were treated with 1V270 (200 μg/injection) on day −1 and 4T1 cells were inoculated on day 0. Three 4T1 cell lysate and 10 units/mL of IL-2 for 3 days. 4T1 and BALB/3T3 cells were labeled with 2.5 μM and 0.25 μM CFSE, respectively, for 12 minutes at 37° C. and were mixed at 1:1 ratio. Splenocytes cultured for 3 days were then cocultured with 4T1 and BALB/3T3 cells at 16:1 to 2:1 effector to target cell ratio (E:T) for 16 hours. The frequencies of 4T1 (CFSE high) and BALB/3T3 (CFSE low) cells were determined by flow cytometry, and the percent specific killing was calculated. Specific killing (%)=[1−“Sample ratio”/“Negative control ratio”)×100; “Sample ratio”=[4T1 (target)/BALB/3T3 (irrelevant)) value of each sample co-cultured with CD8⁺ T cells; “Negative control ratio”=14T1 (target)/BALB/3T3 (irrelevant)) value cultured without CD8⁺ T cells.

### TCR Repertoire Analysis

CD8⁺ T cells were isolated from single cell suspensions of tumors, or spleens using mouse CD8⁺ T cell isolation kit (Miltenyi Biotec). Total RNA was extracted from CD8⁺ T cells with RNeasy Mini Kit (Qiagen, Hilden, Germany) according to the manufacturer's instructions. Next-generation sequencing was performed with an unbiased TCR repertoire analysis technology (Repertoire Genesis Inc., Osaka, Japan) as described previously (Sato-Kaneko et al., 2017).

### Animal Models and Ethic Statement

In the spontaneous metastasis model, othotopically implanted tumor cells spontaneously form lung metastases within three weeks after implantation. 5×10⁵ 4T1 cells were inoculated in both sides of the 4th mammary pads of female BALB/c mice. Tumor length and width were recorded, and tumor volumes were calculated using the formula: volume (mm³)=(width)²×length/2. On day 28, mice were euthanized and the lungs were stained with intratracheally injected India ink and destained in Fekete's solution to count tumor nodules. In the experimental metastasis model, 2×10⁴ 4T1 cells were i.v. injected. On day 21, mice were euthanized and tumor nodules in lungs were counted as described above. In the secondary challenge experiment, 1V270 treated-mice without tumor signals in the lungs on day 21 of the experimental metastasis protocol were included. 5×10⁵ 4T1 cells were inoculated in both sides of the 4th mammary p ads. Tumor length and width were recorded, and tumor volumes were calculated similar with

spontaneous metastasis model.

### Histologic Analysis

Lungs were fixed with 10% formalin for overnight, dehydrated and embedded in paraffin on the Excelsior ES tissue processor (Thermo Scientific) and sectioned at 5 um thickness on a rotary microtome. Antigen retrieval was performed in Citrate buffer, pH 6.0, heated to 98° C. for 8 minutes and cooled at room temperature for 20 minutes. The sections were stained on a Lab Vision 360 automated immunostaining instrument (Thermo Scientific) using a 2 step immunoperoxidase protocol. Briefly, the slides were blocked for endogenous peroxidase activity, washed and incubated with protein blocking buffer, 5% normal donkey serum in TBS for 10 minutes. The primary antibodies, both rabbit poly clonal, CD45 (ab10558, AbCam, used at 1 ug/ml diluted in 5% NDS) and CD3 (ab16669 from AbCam at a 1:100 dilution) were incubated for 1 hour at room temperature. After washing in TBS-tween, the slides were incubated with the secondary antibody—HRP-Donkey F(ab′)2 anti rabbit (Jackson ImmunoResearch Laboratories diluted 1:200) for 30 minutes at room temperature, washed and reacted with DAB as the brown color substrate, hematoxylin was used the counterstain. Antibody details are shown in Table 2. Images were acquired using Axio Imager Zeiss microscope (Zeiss, Thornwood, N.Y.).

TABLE 2
Antibodies used for immunofluorescent staining

| Primary | | | |
|---|---|---|---|
| antibody | Cat# | Host/Isotype | Vendor |
| CD45 | ab10558 | rabbit, poly clonal | abeam |
| CD3 | ab16669 | rabbit, poly clonal | abeam |

| Secondary | | | |
|---|---|---|---|
| antibody | Cat# | Source | Vendor |
| HRP-F(ab′)2 | 711-036-152 | Donkey, | Jackson |
| anti rabbit | | poly clonal | ImmunoResearch |
| | | | Laboratories |

### Cytokine Analysis

The sera were collected from mice receiving 200 nmol of 1V136, 1V270 or vehicle 2, 4, 6, and 24 hours after i.p. administration (15). Cytokine levels in sera were determined by Luminex bead assays (MILLIPLEX™ MAP kit, Millip ore, Billerica, Mass.).

### Flow Cytometric Analysis

To make the single cell suspensions, tumors were dissociated using the mouse tumor dissociation kit (MiltenyiBiotec, San Diego, Calif.) and the gentle MACSOcto Dissociator according to the manufacture's protocol. Single cell suspensions of spleens, lungs and mLN were prepared in Hank's Balanced Salt Solution (HBSS) supplemented with 20 μg/mL DNaseI (Worthington, Lakewood, N.J.) and 0.6 mg/mL collagensse type I (Worthington). Total cell number was counted by the ViaCount assay (MilliporeSigma, Darmstadt, Germany). Dead cells were excluded by propidium iodide staining.

### NK Cells or CD8⁺ Cell Depletion In Vivo

For NK cell depletion, 50 μL of anti-asialo GM1 rabbit poly clonal antibody (Wako, Richmond, Va.) or rabbit IgG polyclonal antibody (Millipore, Temecula, Calif.) was injected on days −1, 1, 5, 9, 13, and 17. Mouse anti-CDS (clone2.43) and isotype control Ab (clone LFA-2) were purchased from BioXcell (West Lebanon, N.H.). Anti-CDS and isotype control (200 μg/dose) were i.p administered on days 5, 8, 11, 14, 16, 19, and 23 to mice.

### Statistical Analysis

Means and standard errors of means (SEM) are shown in other analyses. In dot plots, each dot represents a tumor, a spleen, or a lymph node from an individual mouse and the horizontal and vertical bars indicate mean and mean±SEM. Mann-Whitney test was used to comp are two groups. Using tumor volumes collected over all time points, two-way repeated measures ANOVA was used to comp are different groups, with pair-wise contrasts made at the final time point using a Bonferroni post hoc test. To comp are cross-sectional outcomes among more than two groups, Kruskal-Wallis tests with Dunn's post hoc test were applied. Correlations between tumor volumes and TCR reportire analysis data were analyzed using a Pearson's correlation test, pooling data across the different treatment groups. Analysis of covariance (sometimes on the log scale) was used to test whether the correlation was mediated by differences among the treatment groups in both mean immune marker level and tumor volumes. p<0.05 were considered statistically significant. Prism 6 (GraphPad Software, San Diego, Calif.) statistical software was used to carry out these analyses.

### Results

Systemic Administration of IV270 Inhibits Spontaneous Lung Metastasis in a CD8⁺ Dependent Manner in a Murine 4T1 Orthotopic Breast Cancer Model

1V270 induces tumor-specific adaptive immune responses when administered intratumorally (Hayashi et al., 2011; Sato-Kaneko et al., 2017). However, recent reports claim that TLR7 activation in the lung can promote primary tumor growth (Cherfils-Vicini et al., 2010; Chatterjee et al., 2014). Thus, it was examined whether systemic 1V270 therapy would also promote tumor-specific adaptive T cell responses and if such responses could restrain pulmonary metastatic disease. For this purpose, the murine 4T1 breast cancer model that exhibits characteristics similar to the human disease, in which orthotopically implanted tumor cells spontaneously metastasize to the lungs (Pulaski and Ostrand-Rosenburg 2001), was employed.

4T1 cells (5×10⁵) were inoculated into the 4th mammary pads on day 0, and 1V270 (20, 80, or 200 μg/injection) was injected intraperitoneally (i.p.) twice a week for three weeks, starting on day 7 (FIG. 22A). Both the primary tumor volumes and the numbers of lung metastases were evaluated on day 27. Systemic 1V270 therapy decreased the number of lung metastases (FIG. 22B). However, the systemic administration did not retard tumor growth at the primary sites of implantation, suggesting that rapid tumor growth could overcome immune restraints (FIG. 28).

To study the possible involvement of cytotoxic T cell immune responses in the anti-metastatic effects of 1V270, CD8⁺ cells were depleted with monoclonal antibody (mAbs) prior to treatment with the TLR agonist (FIGS. 22C and 28B). In both the 1V270-treated and the vehicle-treated control mice, the numbers of lung nodules increased significantly (p<0.005) after CD8⁺ cell depletion did not alter the brisk tumor growth at the primary sites in the mammary glands (FIG. 28C).

Systemic Administration of JV270 Induces Tumor-Specific CD8⁺ T Cells in an Intravenous Metastatic Model of 4T1 Breast Cancer

Intravenous (IV) lung metastasis models have been used to evaluate in more detail the immune responses to circulating tumor cells induced by 1V270 therapy. Each animal received 2×10⁴ 4T1 cells directly in the tail vein on day 0, and the numbers of lung nodules were counted on day 21 (FIG. 23A). Similar to the results after orthotopic tumor inoculation, 1V270 inhibited lung metastasis in a dose-dependent manner, with the lowest effective dose being 20 μg (FIG. 23B). A subsequent study indicated that a single administration of 1V270 was sufficient to inhibit lung metastasis and that 1V270 had to be given 1 day prior to the tumor inoculation, which is important for maximal drug effects in this quickly developing lung metastasis model (FIG. 23C).

To examine the role of CD8⁺ T cells after systemic 1V270 treatment, mediastinal lymph node (mLN) cells, splenocytes, and lung tissues were analyzed in the IV metastasis model on day 21 (FIGS. 23D-2G). Activation of CD8⁺ T cells was assessed by intracellular staining for granzyme B and interferon γ (IFNγ). Significantly higher percentages of granzyme Band IFNγ positive CD8⁺ T cells were detected in the 1V270-treated mice (p<0.05, FIGS. 2D and 2E). Histological analysis revealed a higher infiltration of CD45⁺CD3⁺cells in the pulmonary tumor microenvironment of 1V270-treated mice in comparison to those of vehicle-treated mice (FIG. 23F). The tumor-specific effector function of the CD8⁺ cells was further evaluated by ex vivo cytotoxic T cell assays (FIG. 23G). Briefly, splenic CD8⁺ T cells from 1V270-treated or vehicle-treated mice were cultured with antigen (4T1 tumor cell lysates) and IL-2, and then were incubated with carboxy fluorescein succinimidyl ester (CFSE)—labeled tumor cells. CD8⁺ T cells from 1V270-treated mice showed significantly higher tumor-specific cytotoxicity at an effector to target cell ratio of 16:1 (p<0.05, FIG. 23G). These results demonstrated that a single administration of systemic 1V270 therapy can inhibit lung colonization by tumors in a CD8⁺ cell-dependent manner and that the administration of 1V270 promotes adaptive CD8⁺ immune responses against tumor cells.

Tumor-Infiltrating T Cells in IV270 Treated Mice Show High Clonalities and Intra- and Inter-Individual Commonality by TCR Repertoire Analysis

Increased clonality of CD8⁺ T cells has been associated with both a positive clinical outcome and immune-related adverse events after immune checkpoint therapy (Ikeda et al., 2017; Dubudhi et al., 2016). Other studies have also indicated that clonal expansion of tumor-specific T cells is a biomarker for suppression of tumor growth (Straten et al., 1998; Kim et al., 2004). Intratumoral treatment with 1V270 induces local expansion an systemic dispersion of oligoclonal tumor-specific T cells by TCR repertoire analysis using next generation RNAseq methodology (Sato-Kaneko et al., 2017). Thus, it was important to determine whether systemic 1V270 therapy also induced oligoclonal expansion of tumor-specific T cells.

To validate that 1V270 therapy induced tumor-specific adaptive immune responses, we monitored the growth of secondarily challenged tumors following prior 1V270 treatment. The mice treated with 1V270 using the IV metastasis protocol were orthotopically re-challenged with 4T1 cells on day 21 (FIG. 24A). We compared the growth of re-challenged tumors between 1V270-treated mice that were not exposed to the tumor (no-tumor exposed), and 1V270-treated and tumor-exposed mice (FIG. 24A). Naive mice were also orthotopically injected with tumor cells as a positive control. The tumor growth was significantly impaired in the mice treated with 1V270 and exposed to the tumor cells compared to the naive mice or 1V270 treated no-tumor exposed mice (p<0.01, FIG. 24B). As expected, there was no difference in the tumor growth between naive mice and 1V270 treated no-tumor exposure mice (FIG. 24B). To examine whether tumor-specific T cells were recruited into the microenvironment of the secondary challenged tumor, CD8⁺ cells in the tumor infiltrating lymphocytes (TILs) were analyzed by flow cytometry (FIG. 24C). Three-fold higher numbers of CD8⁺ T cells were detected in the TILs from the 1V270-treated and tumor-exposed mice, compared to the 1V270-treated and no-tumor exposed mice (p<0.05, FIG. 24C). In this experiment, more than 80% of CD8⁺ T cells in TILs were positive for PD-1 (FIG. 24C), indicating that they were exposed to antigen (tumor cells) and activated (Fernandez-Poma et al., 3027; Yoshida et al., 2000).

To examine clonal specificity of tumor-specific T cells, CD8⁺ cells were isolated from the spleens and the TILs of secondarily challenged tumors after initial 1V270 therapy. The TCR repertoires were assessed by next generation RNA sequencing of both TCRα and TCR β genes as previously described (Yoshida et al., 2000). The clonality indices of CD8⁺ T cells in TILs, as assessed by I-Shannon index, were negatively correlated with the volumes of the secondarily challenged tumors only in the mice treated with 1V270 and exposed to tumor cells (Pearson's correlation coefficient, r²=0.97, P=0.015, FIGS. 24D and 30A). When the frequencies of commonly shared TCR clones between TILs and splenocytes were evaluated using a binary similarity measure (Baroni-Urbani and Buser overlap index, BUB index) (Zhang et al., 2017), the 1V270-treated and tumor-exposed mice showed significantly higher BUB indices than the control non-tumor exposed mice (p<0.05, FIG. 24E). The clonotypes in individual tumor-exposed mice showed a higher similarity in the 1V270 treated group in comparison to the non-tumor exposed group (FIGS. 24F and 3G). Indeed, 31 TCRα and 11 TCRβ clonotypes were shared by 3 or more individuals in the tumor-exposed group, but only 5 TCRα clonotypes were shared in the non-tumor group (FIG. 30B). The frequency of shared clones was significantly higher in tumor-exposed mice than in non-tumor exposed mice (FIG. 30C). These experiments showed that 1V270 therapy increased clonality of CD8⁺ T cells and the frequency of intra- and inter-individual shared clones, which correlated with the growth inhibition of secondarily challenged tumors.

Dendritic Cells in the Lungs and Draining Lymph Nodes are Activated, and CD S+ T Cells are Recruited to the Draining Lymph Nodes Following 1V270 Therapy

Previously it was demonstrated that 1V270 activates antigen presenting cells (APCs) and promotes cross-presentation of antigen to CD8⁺ T cells (Goff et al., 2015). Since the 1V270 therapy induced a tumor-specific CD8⁺ T cell response in the 4T1 model, we evaluated whether the therapy activated APCs in the lungs, and/or in the draining lymph nodes. BALB/c mice were i.p. administered with 1V270 on day −1, and 4T1 cells were injected the next day, and the dendritic cell populations in the draining mediastinal LNs (mLNs) and the lungs were examined on day 7 after the tumor injection. In the 1V270-treated mice, a population of CD11c⁺ dendritic cells was increased in both mLNs and the lungs (p<0.01, FIGS. 25A and 31B). Furthermore, the frequencies of CD11⁺ cells expressing co-stimulatory molecules (CD80 or CD86) were also significantly increased in mLN at both sites (p<0.01, FIGS. 25B and 31C). The activation of the dendritic cells was accompanied by the recruitment of CD8⁺ T cells with memory and nalve phenotypes (FIG. 25C); central memory CD8⁺ T cells (CD8⁺CD44⁻CD62L⁺), effector memory CD8⁺ T cells (CD8⁺CD44⁻CD62L⁺), and naive CD8⁺ T cells (CD8⁺CD44⁻CD62L⁺) (p<0.05 and p<0.01, FIG. 25C). These findings demonstrated that 1V270 activated local APCs and promoted their maturation, leading to expanded CD8⁺ T cell subsets.

1V270 Treatment Activates Innate Immunity and Inhibits Lung Colonization by Tumor Cells in a NKc-Cell Dependent Manner

In the IV metastasis model, the administration of 1V270 one day before IV injection of tumor cells was required to restrain lung colonization. Since adaptive immune responses require several days to develop, this observation indicated that one or more innate immune cell types in the lung mediated the early therapeutic effect. To enable the monitoring of the detailed kinetics of the colonization process of 4T1 cells expressing both green fluorescent protein (GFP) and luciferase (4T1-GLF) were prepared using lentivirus vectors (Godebu et al., 2014). Subsequently, tumor implantation and growth were monitored using an IVIS Spectrum® in vivo imaging system. In both vehicle- and 1V270-treated mice, tumor cells accumulated in the lungs quickly after the injection (at Oh, FIGS. 25D and 32B). Thereafter, the signals progressed in two phases; they declined almost to baseline within the first 24 hours (the early phase) and then increased after day 7 (the late phase,). The 1V270 treatment significantly suppressed the tumor signals both in the early (p<0.01 on 3 hours and 6 hours) and in the late phases (p<0.05 on days 7, 10, and 14).

To identify the types and functions of innate immune cells recruited into the lung following 1V270 administration, the mice were injected with 1V270 on day −1, 4T1 cells were i.v. administered on day 0, and the bronchioalveolar cells were isolated on day 7 after the tumor injection. A single cell suspension of lung cells was stained for natural killer (NK) cells and myeloid suppressor cells (MDSC) and analyzed by flow cytometry (FIGS. 33A-C and Table 1). Significantly increased populations of NK cells, and monocytic-MDSC were recruited to the lungs following 1V270 treatment, in comparison to the vehicle treatment (p<0.01, FIGS. 25E and 33D). On the other hand, similar populations of granulocytic (PMN)MDSCs accumulated in the lungs in the 1V270-treated mice compared to those of the vehicle-treated animals (FIG. 33D).

NK cells can be activated directly by TLR7 agonists, and indirectly by type I IFN which is secreted from accessory dendritic cells (Liu et al., 2007; Hart et al., 2005). To examine the role of NK cells in the early therapeutic efficacy of 1V270, this cell type was depleted by treatment with anti-asialo GM1 poly clonal antibody (Kasai et al., 1980). Over 90% of NK cells were depleted by antibody injection on days −4, −1, 3 and 10 (FIGS. 34A-B). IVIS analysis showed that lung colonization by tumor cells in 1V270 treated NK cell-depleted mice during the early phase developed similar to that in vehicle treated mice (FIGS. 25F and 34C). These data indicate that NK cells played a therapeutic role in the early phase.

Intranasal Administration of 1V270 is Also Effective in Preventing Metastasis and in Inducing Anti-Tumor Immunity

It was demonstrated that intranasal (i.n.) administration of 1V270 activates nasal and lung APCs, without causing systemic cytokine release (Wu et al., 2014). Therefore, we examined whether in 1V270 treatment could impair tumor growth in the IV metastasis model (FIG. 26A). 1V270 (20 or 200 μg/50 μL dose) was in, delivered on days −3, −1, 3, 7, and 10 relative to the 4T1 tumor cell injection (day 0) (FIG. 26A). Intranasally administrated 1V270 inhibited the number of lung nodules in a dose-dependent manner (FIG. 26B).

It was examined whether i.n. administration of 1V270 could induce tumor-specific adaptive immune responses similar to the effects of systemic administration. Mice were treated with i.n. 1V270 (200 or 500 μg/50 μL) and then received 4T1 cells by the i.v. route. The surviving mice on day 21 were orthotopically re-challenged with tumor cells. The re-challenged tumor growth was significantly inhibited in the mice which were i.n. treated with 1V270 (FIG. 26C). The tumors grew similarly in narve mice when the mice received 1V270 treatment intranasally without tumor cell injection (FIG. 26C). A higher number of CD8⁺ T cells and PD-1⁺CD8⁺ T cells were detected in TILs from i.n. 1V270-treated mice exposed to the tumor cells, in comparison to 1V270 treated without tumor exposure or nafve mice (FIG. 26D). These findings indicate that i.n. delivery of 1V270 effectively inhibited lung metastasis by inducing tumor-specific adaptive immune responses, similar to systemic 1V270 therapy.

Anti-Metastatic Effects of JV270 were Observed in Murine Syngeneic Melanoma and Lung Carcinoma Models

To evaluate whether the 1V270 therapy can be effective in other cancer types, we employed two additional murine syngeneic metastasis models; B16 melanoma and Lewis lung cancer (LLC). Luciferase and GFP expressing cells (B16-GLF and LLC-GLF) were prepared using a lentivirus vector for in vivo imaging analysis. Mice received systemic 1V270 treatment on day −1, and then B16-GLF and LLC-GLF cells were i.v. administered on day 0. In both metastasis models, 1V270 inhibited lung metastasis by day 14 (FIGS. 27A and 27E) and prolonged mouse survival (FIGS. 27B and 27F).

### Discussion

In patients with an advanced stage of cancer, the development of metastasis is almost inevitable since the metastatic niches are seeded with tumor cells long before clinical presentation (Valastyan and Weinberg, 2011). The ability of immune-checkpoint inhibitors to reactivate tumor-specific cytotoxic T cells provided evidence that immunotherapy can overcome these limitations, at least in some patients. Thus, there is an unmet medical need for additional agents that can increase the frequency of cytotoxic T cells at metastatic sites. Because each immunotherapy type exploits a distinct biological mechanism, biomarkers that predict efficacy and adverse effects are required (Topalian et al., 2000). Systemic 1V270 treatment systemically induced cytotoxic tumor-specific CD8⁺ T cells, as assessed by both in vitro tumor-specific cytotoxicity assays, and tumor re-challenge experiments. TCR repertoire analyses of TILs in the secondarily challenged tumors indicated that 1V270 therapy strongly increased T cell clonality. The levels of clonality negatively correlated with the tumor volumes of secondarily challenged tumors. Of interest, the clonal similarity between tumor infiltrating and splenic T cells was increased in 1V270 treated and tumor-exposed animals. A recent paper demonstrated that antitumor immune cells proliferate in the secondary lymphoid organs, including draining LNs and spleen, and can be detected in the peripheral blood during tumor rejection (Spitzer et al., 2017). These findings suggested that immune monitoring should be possible by analyzing the TCR repertoire of peripheral T cells.

Theoretically, the TCR repertoire might be diverse among individual tumor-bearing mice, even though they share the same genetic background (Venturi et al., 2008). In a chronic virus infection, patients develop common clones which interact with highly immunodommant antigens (Cerundolo et al., 2016; Miyama et al., 2017). In the present study, an eight-fold higher number of shared clones in TILs was identified in the 1V270 treated and tumor-exposed group, compared to the no-tumor exposed group. As increased frequency of shared clones suggested that the systemic 1V270 treatment may skew the TCR repertoire toward tumor-specific clones, that may recognize the same tumor antigens.

When administrated locally, synthetic TLR7 and TLR9 agonists are potent immune adjuvants, that can induce Th1 and cytotoxic T cell responses over a period of week (Sato-Kaneko et al., 2017; Cho et al., 2002). When given systemically, however, some TLR agonists can cause a cytokine release syndrome that could potentially enhance metastatic growth by stimulating either angiogenesis or the development of M2 macrophages (Hageman et al., 2005; Sanmarco et al., 2017). Therefore, effective systemic TLR7 therapy must clearly demonstrate that CD8 responses are induced without toxicity to the host or adverse changes in the tumor microenvironment. The present data demonstrated that some monocyte linage, myeloid derived suppressor cells (MDSCs) were recruited to the lung after 1V270 administration. Immature MDSCs have the ability to suppress anti-tumor T cell responses (Quail and Joyce, 2013). Thus, we were concerned that systemic 1V270 treatment may promote tumor growth. However, other innate immune cells, including NK cells and dendritic cells, were also recruited to the lung after 1V270 administration, as reported previously in other models using TLR ligands (Smits et al., 2008). The recruitment and activation of the NK cells impeded tumor lung colonization, indicating that the NK cells could overcome the suppressive function of MDSC recruited by 1V270 administration. Another concern in immunotherapy using TLR7 ligands is that stimulation of a tumor TLR7 pathway could promote growth and chemo-resistance in some primary tumors expressing this receptor (Chrfils-Vincini et al., 2010; Chatterjee et al., 2014). In our study, 4T1, B16, and LLC cells did not express TLR7 by quantitative RT-PCR (Supplemental figures 8). We therefore conclude that systemic and i.n. TLR7 treatment is an effective therapy for TLR7 negative tumors.

1V270 inhibited the growth of small subcutaneous tumors when locally (intratumorally) injected (Hayashi et al., 2011; Sato-Kaneko et al., 2017). In the 4T1 metastatic model, orthotopically implanted primary tumors in the mammary gland were advanced at the time of initiation of 1V270 treatment. The ability of the TLR7 phospholipid agonist to prevent early lung metastasis may be attributable both to the lower tumor burden and to NK recruitment and activation. The in vivo imaging studies in NK cell depleted mice confirmed the critical role of this cell type in constraining tumor colonization, thus allowing for the development of a specific CD8 T cell response in the later phases of metastasis.

Intratracheal administration of a low molecular weight TLR7 agonist (SM276001) was reported to suppress metastatic lung tumors (Koga-Yamakawa et al., 2013). It was demonstrated that a low molecular weight TLR 7 agonist (SM360320)(1V136) induced high levels of systemic proinflammatory cytokines following parenteral and i.n. administration to mice and that conjugation of a TLR7 ligand to a phospholipid moiety could markedly reduce in vivo cytokine release (FIG. 36) (Chan et al., 2009). Intranasally administered 1V270 induced local (lung) immune cell activation without inducing systemic cytokine release (Wu et al., 2014). These results prompted us to assess whether the drug might have an immunotherapeutic effect in pulmonary metastatic disease. Our data demonstrated that i.n. administration of 1V270 suppressed lung metastasis similar to parenteral inoculation (FIG. 26), suggesting that i.n. delivery might be clinically attractive for this drug.

In summary, single systemic administration of a phospholipid conjugated TLR7 agonist inhibited lung metastasis in three different murine syngeneic models of human malignancy, 4T1 breast cancer, B16 melanoma and Lewis lung carcinoma models. The drug quickly activated NK cells in the lung, and later induced a cytotoxic T cell response. These two different mechanisms, NK cell-mediated and tumor-specific adaptive T cell responses, were responsible for the early and late phases of tumor growth inhibition. The anti-tumor effects were achieved without significant systemic release of inflammatory cytokines following systemic administration. Furthermore, 1V270 therapy induced oligoclonal CD8 T cell responses as determined by TCR repertoire analyses of both spleen and mediastinal lymph nodes. The emergence of shared T cell clones correlated with the development of adaptive immunity against tumor cells. These results suggest that TCR repertoire analyses may be used to guide clinical trials of TLR and other immunotherapies in patients with metastatic cancer.

### REFERENCES

Barry et al., Nature, 493:106 (2013).
Brahmer et al., N. Engl. J. Med., 373:123 (2015).
Camargo et al., Curr. Biol., 17:2054 (2007).
Cerundolo et al., J. Immunol. Ref., 175:6123 (2016).
Chan et al., Hioconjug. Chem., 20:1194 (2009).
Chatterjee et al., Cancer Res., 74:5008 (2014).
Cherfils-Vicini et al., J. Clin. Invest., 120:1285 (2010).
Chiappinelli et al., Cell, 162:974 (2015).
Cho et al., J. Immunol., 168:4907 (2002).
Cottini et al., Nat. M ed., 20:599 (2014).
Dajon et al., Oncoimmunology, 4:c991 (2015)
Dewey et al., Curr. Biol., 25:2751 (2015).
Dong et al., Cell, 130:1120 (2007).
Dranoff, Nat. Rev. Immunol., 12:61 (2011).
Engel et al., Expert Rev. Clin. Pharmacol., 4:275 (2011).
Fernandez-Poma et al., Cancer Res., 77:3672 (2017).
Fuertes et al., Trends Immunol., 34:67 (2013).
Gajewski et al., Nat. Immunol., 14:1014 (2013).
Geisse et al., J. Am. Acad. Dermatol., 50:722 (2004).
Godebu et al., Transl. Med., 12:27.1 (2014).
Goff et al., J. Virol., 89:3221 (2015).
Hagemann et al., J. Immunol., 175:1197 (2005).
Hanahan and Weinberg, Cell, 144:646 (2011).
Hansen et al., Trends Cell Biol., 25:499 (2015).
Hart et al., J. Immunol., 175:1636 (2005).
Harvey et al., Nat. Rev. Cancer, 13:246 (2013).
Hayashi et al., Melanoma Reseach., 21:66 (2011).
Ikeda et al., Oncol. Rep., 37:2603 (2017).
Johnson and Halder, Nat. Rev. Drug Discov., 13:63 (2014).
Junt and Barchet, Nat. Rev. Immunol., 15:529 (2015).
Kasai et al., Eur. J. Immunol., 10:175 (1980).
Keder et al., Curr. Biol., 25:2739 (2015).
Kim et al., Surgery, 136:295 (2004).
Kitamura et al., Nat. Rev. Immunol., 15:73 (2015).
Koga-Yamakawa et al., Int. J. Cancer, 132:580 (2013).
Komurov et al., BMC Genomics, 13:282 (2012).
Lambert et al., Cell, 168:670 (2017).
Lei et al., Onco Targets Ther., 9:545 (2016).
Liu et al., Blood, 109:4336 (2007).
Liu et al., Cell, 164:406 (2016).
Meng et al., Genes Dev., 30:1 (2016).
Meng et al., Nat. Commun., 6:8357 (2015).
Mi et al., Nat. Protoc., 8:1551 (2013).
Miyama et al., Sci. Rep., 7:3663 (2017).
Mizuno et al., BMC Med. Genomics, 2:18 (2009).
Mohammed et al., J. Thorac. Imaging 1:26 (2011).
Mori et al., Cell, 156:893 (2014).
Moroishi et al., Genes Dev., 29:1271 (2015b).
Moroishi et al., Nat. Rev. Cancer, 15:73 (2015a).
Moya and Halder, Cell Res., 24:137 (2014).
Nowell et al., Nat. Cell Biol., 18:168 (2016).
Ochi and Graffeo, J. Clin. Invest., 122:4118 (2012).
Ostraml-Rosenberg and Fenselau, J. Immunol., 200:422 (2018).
Pico de Coana et al., Trends Mol. Med., 21:482 (2015).
Pulaski et al., Curr. Protoc. Immunol., 20:20.2 (2001).
Quail and Joyce, Oncologist, 13:859 (2008).
Ran et al., Nat. Protoc., 8:2281 (2013).
Robbins and Morelli, Nat. Rev. Immunol., 14:195 (2014).
Sanmarco et al., Biochim Biophys. Acta-Mn Basis Dis., 1863:R57 (2017).
Sato-Kaneko et al., Insight, 2 (2017).
Savage et al., Br. T. Cancer, 74:1482 (1996).
Schwanhausser et al., Nature, 473:337 (2011).
Senovilla et al., Science, 337:1678 (2012).
Sharma and Allison, Cell, 161:205 (2015).
Sharma and Allison, Cell, 161:205 (2015).
Sistigu et al., Nat. Med., 20:1301 (2014).
Spitzer et al., Cell, 168:487 (2017).
Steeg Nat. Rev. Cancer, 16:201 (2016).
Straten et al., Proc. Natl. Acad. Sci. U.S.A., 95:8785 (1998).
Subudhi et al., Proc. Natl. Acad. Sci., 2:11919.
Taniguchi et al., Nature, 519:57 (2015).
Topalian et al., Nat. Rev. Cancer, 16:275 (2016).
Valastyan and Weinberg Cell, 147:275 (2011).
Venturi et al., Nat. Rev. Immunol., 8:231 (2008).
Wang et al., Mol. Cancer Ther., 9:1788 (2010).
Wang et al., Cancer Metastasis Rev., 33:173 (2014).
Wolchok et al., N. Engl. J. Med., 369:122 (2013).
Wolchok et al., N. Engl. J. Med., 377:1345 (2017).
Wu et al., Innate Immun., 6:315 (2014).
Yanez-Mo et al., J. Extracell. Vesicles 4:27066 (2015).
Yoshida et al., Immunogenetics, 52:35 (2000).
Yu et al., Cell, 163:811 (2015).
Zhang et al., BMC Bioinformatics., 18:129 (2017).
Zhao et al., Genes Dev., 21:2747 (2007).
Zhao et al., Genes Dev., 22:1962 (2008).
Zhou et al., Cancer Cell, 16:425 (2009).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A method to isolate and expand cancer-specific CD8+ T cells, comprising:
administering to a mammal having a tumor an effective amount of a composition comprising an agent that promotes the release of extracellular vesicles from tumor cells; and
collecting from the mammal extracellular vesicles and immune cells including tumor specific CD8+ T cells.

2. The method of claim 1 wherein the composition comprises a TLR7 or TLR9 agonist.

3. The method of claim 1 wherein the extracellular vesicles and the immune cells are collected from blood of the mammal.

4. (canceled)

5. The method of claim 1 wherein the agent inhibits or inactivates LATS1 and/or LATS2.

6. The method of claim 1 further comprising culturing the immune cells to expand and/or activate cancer-specific CD8+ T cells.

7. (canceled)

8. The method of claim 1 wherein the collected extracellular vesicles are less than about 0.5 microns in diameter.

9. The method of claim 3 wherein the collected extracellular vesicles are isolated.

10-13. (canceled)

14. The method of claim 6 further comprising collecting the expanded, or activated and expanded, cancer-specific CD8+ T cells.

15. The method of claim 1 wherein the composition is orally or parenterally administered or by intrapulmonary routes.

16. The method of claim 1 wherein the composition is administered to the tumor by direct injection or wherein the composition is administered systemically using liposomes, antibodies or other targeting mechanisms.

17-18. (canceled)

19. A method to isolate and expand cancer-specific CD8+ T cells, comprising:
collecting from a mammal having a tumor extracellular vesicles and immune cells including tumor specific CD8+ T cells;
culturing the immune cells and enriching for cancer-specific CD8+ T cells; and
culturing the enriched cancer-specific CD8+ T cells and the extracellular vesicles.

20. The method of claim 19 wherein the mammal is subjected to chemotherapy radiation therapy, immunotherapy or targeted therapy before or after the immune cells are collected.

21. The method of claim 19 wherein the mammal is subjected to chemotherapy, radiation therapy, immunotherapy or targeted therapy before and after the immune cells are collected.

22. The method of claim 19 further comprising isolating the cultured cancer-specific CD8+ T cells.

23. The method of claim 6 wherein the cancer specific CD8+ T cells are administered to the mammal.

24. The method of claim 23 wherein the mammal is subjected to chemotherapy, radiation therapy, immunotherapy or targeted therapy after the cancer-specific CD8+ T cells are administered.

25. The method of claim 20 wherein the therapy is anti-PD therapy.

26. The method of claim 20 wherein the therapy is a checkpoint inhibitor therapy.

27. The method of claim 26 wherein the inhibitor comprises pembrolizumab, nivolumab, atezolizumab, avelumab, durvalumab, or ipilimumab.

28. A method to enhance the immunogenicity of tumor cells, comprising:
modifying ex vivo tumor cells of a mammal to provide for tumor cells that do not express or have reduced expression of LAT1 and/or LAT2; and
administering to the mammal an amount of the modified cells effective to enhance the immune response to the tumor in the mammal.
